(19) United States
(12) Patent Application Publication
(10) Pub. No.: US 2023/0295324 A1
(43) Pub. Date: Sep. 21, 2023

(54) OX40-TARGETED ANTIBODY, AND PREPARATION METHOD THEREFOR AND APPLICATION THEREOF

(71) Applicant: HARBOUR BIOMED (SHANGHAI) CO., LTD, Shanghai (CN)

(72) Inventors: Lei SHI, Shanghai (CN); Bing HUANG, Shanghai (CN); Jian WANG, Shanghai (CN); Xingxing JIA, Shanghai (CN)

(21) Appl. No.: 18/013,707

(22) PCT Filed: Jun. 29, 2021
(86) PCT No.: PCT/CN2021/102946

§ 371 Date: Dec. 29, 2022

(30) Foreign Application Priority Data
Jun. 30, 2020 (CN) 202010618134.5

(51) Int. Cl. C07K 16/28; C07K 16/30; C07K 14/725; A61K 47/68; A61K 45/06; A61P 35/00; C12N 15/85; G01N 33/574

(52) U.S. Cl. CPC C07K 16/2878; C07K 16/2827; C07K 16/3069; C07K 16/30; C07K 16/28; C07K 14/7051; A61K 47/6849; A61K 45/06; A61P 35/00; C12N 15/85; G01N 33/57492; C07K 2317/31; C12N 2800/107; G01N 2333/70596

(57) ABSTRACT

An OX40-targeted antibody or an antigen binding fragment thereof. The OX40-targeted antibody comprises a VH. The VH comprises the following CDRs or mutants thereof: a VH CDR1 as shown in an amino acid sequence of SEQ ID NO: 10, a VH CDR2 as shown in an amino acid sequence of SEQ ID NO: 44, and/or a VH CDR3 as shown in an amino acid sequence of SEQ ID NO: 86, SEQ ID NO: 84, or SEQ ID NO: 89. Also disclosed is a preparation method, an application, and a double-antibody comprising same. The antibody or the antigen binding fragment thereof can specifically bind to OX40, promote larger activation of NF-Kb, activate OX40 pathways in vitro, and induce activation of T cell function. The double-antibody can identify tumor target TAA, can bind to OX40 on the T cell, and can recruit and activate T cells near tumor cells to kill tumor cells.

### CROSS-REFERENCE TO RELATED APPLICATIONS

This application is a U.S. National Phase Application under 35 U.S.C. 371 of International Application No. PCT/CN2021/102946, filed on Jun. 29, 2021, which claims the benefit of Chinese Patent Application No. 202010618134.5 filed on Jun. 30, 2020. The entire disclosures of the above applications are incorporated herein by reference.

### SEQUENCE LISTING

The Sequence Listing is submitted concurrently with the specification as an ASCII formatted text file via EFS-Web, with a file name of “17424-149_SequenceListing.txt”, a creation date of Dec. 21, 2022, and a size of 351,000 bytes. The Sequence Listing filed via EFS-Web is part of the specification and is incorporated in its entirety by reference herein pursuant to 37 C.F.R. § 1.52(e)(5).

### TECHNICAL FIELD

The present application relates to the field of biomedicine, in particular to an OX40-targeted antibody or an antigen-binding fragment thereof, a method for preparing the same and use of the same, and to a bispecific antibody comprising the OX40-targeted antibody or the antigen-binding fragment thereof.

### BACKGROUND

OX40, also known as CD134 or tumor necrosis factor receptor superfamily member 4 (TNFRSF4), is one of the tumor necrosis factor receptor superfamily members (TNFRSF). It is a type 1 transmembrane glycoprotein of 50-55 kDa, and has an intracellular domain, a transmembrane domain, and an extracellular domain. It is involved in enhancing T cell responses triggered by T cell receptors (TCRs), and is a costimulatory receptor molecule. After TCR stimulation, OX40 is predominantly expressed on the surface of activated CD4⁺ and CD8⁺ T cells, with higher expression on CD4⁺ T cells in vitro and at the tumor site than on CD8⁺ T cells (Fujita T et al., (2006) Immunol Lett. 106(1): 27-33; Montler R et al., (2016) Clin Transl Immunology. 5 (4): e70). Furthermore, studies have shown that regulatory T cells (Tregs) express more OX40 than conventional CD4⁺ T cells in a variety of human tumors (Timpori E et al., (2016) Oncolmmunology.; 5(7): e1175800; Piconese S et al., (2014) Hepatology. 60(5): 1494-1507), so there is also the possibility of preferentially targeting OX40hi population with depleting mAbs. OX40 has also been reported to be expressed on human neutrophils (this signaling pathway supports survival) and murine natural killer (NK) and NK T cells (Baumann Ret al., (2004) Eur J Immunol. 34(8): 2268-2275; Croft et al., (2009) Nat Rev Immunol. 9(4): 271-285).

The only ligand known to OX40 is OX40L (TNFSF4), which is a type II transmembrane protein containing a conserved tumor necrosis factor (TNF) homeodomain that enables trimerization (Bodmer J L et al., (2002) Trends Biochem Sci. 27(1): 19-26). After activation, three OX40 molecules can bind to an OX40L trimer, which is a typical feature of ligand-receptor pairing in TNFRSF (Banner D W et al., (1993) Cell. 73(3): 431-445). After exposure to thymic stromal lymphopoietin, OX40L was induced on human dendritic cells (DCs) (Krause P et al., (2009) Blood. 113(11): 2451-2460). Furthermore, human monocytes, neutrophils, mast cells, lymphoid tissue-inducer cells, smooth muscle cells, endothelial cells and B cells activated in vitro all express OX40L under appropriate conditions (Byun M et al, (2013) J Exp Med. 210(9): 1743-1759; Karulf M et al., (2010) J Immunol. 185(8): 4856-4862).

For OX40, transmembrane signaling is mediated primarily through members of the TNF receptor-associated factor (TRAF) family, as are other members of the TNFRSF. TRAF is a trimeric protein that interacts with short motifs in the cytoplasmic tail of the ligand-bound TNFRSF receptor trimer (McWhitter S M et al., (1999) Proc Natl Acad Sci USA. 96(15): 8408-8413; Park Y C et al., (1999) Nature. 398(6727): 533-538). For OX40, the receptor-ligand interaction stimulates OX40 and TRAF2 to enter the cytoplasm to activate downstream signaling pathways mediated by PI3K/PKB, NF-κB and NFAT, thereby activating the division and survival of T cells and production of cytokines (Croft et al, (2009) Nat Rev Immunol. 9(4): 271-285.; Watts, (2005) Annu. Rev. Immunol. 23, 23-68). Thus, both CD4⁺ and CD8⁺ T cells are potential targets of OX40-directed immunotherapy of cancer.

There are currently a number of different OX40-targeted molecules used in clinical trials for metastatic cancer, one of which is OX40L-Fc fusion protein MEDI6383, currently in clinical phase I. The others are agonistic antibodies, such as OX40 antibodies MEDI6469 (9B 12, mouse anti-human OX40 mAb, replaced by MEDI0562 humanized mAb), MEDI0562 (AstraZeneca, tavolixizumab), ivuxolimab (Pfizer, PF-04518600), GSK3174998 (GSK), BMS-986178 (BMS), and Pogalizumab (MOXRO0916/RG7888). Among them, Pogalizumab, also known as vonlerolizumab, is a humanized OX40 antibody developed by Roche, which is currently in clinical phase II for the treatment of solid tumors. Some preclinical studies have shown that anti-OX40 monoclonal antibodies produce deleterious immunosuppressive side effects by promoting MDSC accumulation and Th2 cytokine production (Gough M J et al., (2012) Immunology 136: 437e47). In addition, although OX40-targeted agonist monoclonal antibodies may confer tumor protection in mice, their effect is limited in less immunogenic environments (Kjaergaard J et al., (2000) Cancer Res. 60(19): 5514-5521).

Heavy chain-only antibodies were reported by Belgian scientists for the first time in Nature in 1993. Heavy-chain antibodies and nanobodies (VHH) are superior to conventional antibodies in many aspects, as they have a small molecular weight, can penetrate blood brain barrier and are less immunogenic to humans. Moreover, the heavy-chain antibodies or nanobodies are particularly suitable for the development of bispecific antibodies, and can solve the problems of light chain mismatch and heterodimerization. There are few reports in the prior art relating to OX40 heavy chain-only antibodies.

Therefore, there is an urgent need to develop safer and more effective combination therapy strategies targeting OX40, such as enhancing antigen availability, enhancing inflammation, or suppressing immunosuppressive signals, for use in the treatment of a variety of cancers.

### SUMMARY

The present invention aims to solve the technical problems to overcome the defects of the current OX40-targeted antibodies by providing an OX40-targeted antibody, a method for preparing the same and use of the same, as well as a bispecific antibody developed based on the antibody and use of the same. The antibody or the antigen-binding fragment thereof of the present invention has activity in specifically binding to human OX40 and cynomolgus monkey (cyno) OX40. In addition, the antibody or the antigen-binding fragment thereof of the present invention can promote greater activation of NF-κb, thereby stimulating the OX40 signaling pathway, and can activate the OX40 pathway in vitro and induce the activation of T cells, with the activation effect comparable to or greater than existing antibodies (e.g., Pogalizumab). Meanwhile, the antibody or the antigen-binding fragment thereof of the present invention has crosslinking dependence of FcγRIIB (CD32B), which is one of the Fcγ receptor members. In the preparation of bispecific antibodies using the antibody or the antigen-binding fragment thereof of the present invention, the resulting bispecific antibodies are all capable of binding to human OX40 and the corresponding tumor-associated antigens, one end of which can recognize a tumor target TAA (e.g., PSMA, EPCAM, CLDN18.2, B7H4, or PD-L1) specifically expressed on the surface of tumor cells, and the other end of which can bind to an OX40 molecule on T cells, and can recruit and activate T cells in the vicinity of tumor cells, thereby killing the tumor cells.

In order to solve the above technical problems, a first aspect of the present invention provides an OX40-targeted antibody or an antigen-binding fragment thereof comprising a heavy chain variable region (VH),

wherein the VH comprises the following complementarity determining regions (CDRs) or mutations thereof: VH CDR1 with an amino acid sequence as set forth in SEQ ID NO: 10; VH CDR2 with an amino acid sequence as set forth in SEQ ID NO: 44; and/or VH CDR3 with an amino acid sequence as set forth in SEQ ID NO: 86, SEQ ID NO: 84 or SEQ ID NO: 89;
wherein the mutation is an insertion, deletion or substitution of 3, 2 or 1 amino acids on the basis of the amino acid sequences of the VH CDR1, VH CDR2 and VH CDR3 of the VH.

In the present application, “amino acid mutation” in the context like “insertion, deletion or substitution of 3, 2 or 1 amino acids” refers to a mutation of an amino acid in the sequence of a variant as compared to the sequence of an original amino acid, including the insertion, deletion or substitution of amino acids on the basis of the original amino acid sequence. An exemplary explanation is that the mutations to the CDRs may comprise 3, 2 or 1 amino acid mutations, and that the same or different numbers of amino acid residues can be optionally selected for the mutations to those CDRs, e.g., 1 amino acid mutation to CDR1, and no amino acid mutation to CDR2 and CDR3.

In the present application, the mutation may include mutations currently known to those skilled in the art, for example, mutations that may be made to an antibody during the production or application of the antibody, for example, mutations at a site where post-transcriptional modifications (PTMs) of, in particular, CDR regions, may be present, including aggregation of antibodies, asparagine deamidation (NG, NS, NH, etc.) sensitive sites, aspartate isomerization (DG, DP) sensitive sites, N-glycosylation (N-{P}S/T) sensitive sites, oxidation sensitive sites, and the like.

Preferably, the VH CDR1 has mutations of F2, T3, S5 and/or S6 to L, S, P, I, D and/or C on the amino acid sequence as set forth in SEQ ID NO: 10, preferably amino acid substitutions of F2L, T3S/P/I, S5D and/or S6D/C on the amino acid sequence as set forth in SEQ ID NO: 10, with an amino acid sequence as set forth in, for example, any one of SEQ ID NOs: 13-16 or SEQ ID NOs: 20-24.

Preferably, the VH CDR2 has mutations of S1, R3, G4, G5 and/or S6 to T, H, L, G, S, N, D, I and/or Q on the amino acid sequence as set forth in SEQ ID NO: 44, preferably 3, 2 or 1 amino acid substitutions of S1T, R3H/L/G/S, G4S, G5N/D and S6N/I/Q/T on the amino acid sequence as set forth in SEQ ID NO: 44, with an amino acid sequence as set forth in, for example, any one of SEQ ID NOs: 42-43, SEQ ID NOs: 45-50 or SEQ ID NOs: 54-60.

Preferably, the VH CDR3 has mutations of T2, T5, T6, D9 and/or Y10 to M, I, V, S, W, Y, C, F and/or W on the amino acid sequence as set forth in SEQ ID NO: 86, preferably 2 or 1 amino acid substitutions of T2M/I/V, T5S, T6W/Y, D9C and Y10F/W on the amino acid sequence as set forth in SEQ ID NO: 86, with an amino acid sequence as set forth in, for example, any one of SEQ ID NOs: 82-83, SEQ ID NO: 85, SEQ ID NOs: 87-88, SEQ ID NO: 90 or SEQ ID NOs: 94-99.

The above F2L generally refers to the amino acid F at position 2 of the amino acid sequence as set forth in SEQ ID NO: 10 mutated to L, and other amino acid substitutions such as T3S/P/I, S5D and/or S6D/C have a definition rule similar to the F2L, which should be understood by those skilled in the art.

Preferably, the OX40-targeted antibody or the antigen-binding fragment thereof comprises a heavy chain variable region (VH), wherein the VH comprises the following complementarity determining regions (CDRs) or mutations thereof: VH CDR1 with an amino acid sequence as set forth in any one of SEQ ID NO: 10, SEQ ID NOs: 13-16 or SEQ ID NOs: 20-24; VH CDR2 with an amino acid sequence as set forth in any one of SEQ ID NOs: 42-50 or SEQ ID NOs: 54-60; and/or VH CDR3 with an amino acid sequence as set forth in any one of SEQ ID NOs: 82-90 or SEQ ID NOs: 94-99.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 13,42 and 82, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10,42 and 83, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 14,42 and 83, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10,43 and 84, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 13, 44 and 82, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 15, 44 and 83, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 44 and 83, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 45 and 83, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 46 and 85, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 44 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 16, 42 and 82, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 47 and 87, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 16, 44 and 83, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 45 and 88, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 48 and 89, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 49 and 90, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 49 and 83, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 50 and 90, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 20, 44 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 21, 44 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 54 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 55 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 56 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 42 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 57 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 58 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 59 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 60 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 44 and 94, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 44 and 95, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 44 and 96, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 44 and 97, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 44 and 98, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 44 and 99, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 54 and 95, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 54 and 97, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 55 and 95, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 55 and 97, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 56 and 95, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 56 and 97, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 22, 54 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 23, 54 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 24, 54 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 21, 54 and 86, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 10, 54 and 99, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 22, 54 and 99, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 23, 54 and 99, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 24, 54 and 99, respectively.

In a certain preferred embodiment, the OX40-targeted antibody or the antigen-binding fragment thereof comprises a heavy chain variable region (VH), wherein the VH comprises VH CDR1, VH CDR2 and VH CDR3 with amino acid sequences as set forth in SEQ ID NOs: 21, 54 and 99, respectively.

Preferably, the above VH further comprises a heavy chain variable region framework region (VH FWR), wherein the VH FWR may, for example, be selected from the germline IGHV3-23 or a back mutation thereof. More preferably, the VH FWR is a heavy chain variable region framework region of a human antibody.

Preferably, the VH comprises an amino acid sequence as set forth in any one of SEQ ID NOs: 142-164 or SEQ ID NOs: 168-198 or a mutation thereof, wherein the mutation is a deletion, substitution or addition of one or more amino acid residues on the amino acid sequence of the VH, and the amino acid sequence with the mutation has at least 85% sequence identity to the amino acid sequence of the VH and maintains or improves the binding of the antibody to OX40, wherein the at least 85% sequence identity is preferably at least 90% sequence identity, more preferably at least 95% sequence identity, and most preferably at least 99% sequence identity.

In the present application, the amino acid sequences of the listed CDRs are all shown according to the Chothia scheme (the sequences in the claims of the present application are also shown according to the Chothia scheme). However, it is well known to those skilled in the art that the CDRs of an antibody can be defined in the art using a variety of methods, such as the Kabat scheme based on sequence variability (see Kabat et al., Sequences of Proteins of Immunological Interest, Fifth Edition, National Institutes of Health (U.S.), Bethesda, Maryland (1991)), and the Chothia scheme based on the location of the structural loop regions (see J Mol Biol 273: 927-948, 1997). In the present application, the Combined scheme comprising the Kabat scheme and the Chothia scheme can also be used to determine the amino acid residues in a variable domain sequence. The Combined scheme combines the Kabat scheme with the Chothia scheme to obtain a larger range, which is detailed in Table a. It will be understood by those skilled in the art that unless otherwise specified, the terms “CDR” and “complementarity determining region” of a given antibody or a region (e.g., variable region) thereof are construed as encompassing complementarity determining regions as defined by any one of the above known schemes described herein. Although the scope claimed in the present invention is the sequences shown based on the Chothia scheme, the amino acid sequences corresponding to the other schemes for numbering CDRs shall also fall within the scope of the present invention.

TABLE a
The definition scheme for the CDRs of the antibody of
the present application (see http://bioinf.org.uk/abs/)

| | Kabat | Chothia | Combined |
|---|---|---|---|
| VH CDR1 | H31--H35 | H26--H32 | H26-H35 |
| VH CDR2 | H50--H65 | H52--H56 | H50-H65 |
| VH CDR3 | H95--H102 | H95--H102 | H95-H102 |

In the table, Haa-Hbb can refer to an amino acid sequence from position aa to position bb beginning at the N-terminus of the heavy chain of the antibody. For example, H26-H32 can refer to an amino acid sequence from position 26 to position 32 beginning at the N-terminus of the heavy chain of the antibody according to the Chothia scheme. It should be known to those skilled in the art that there are positions where insertion sites are present in numbering CDRs with the Chothia scheme.

For example, as defined by the Chothia scheme, the VH comprises CDRs described in table b below.

TABLE b

| Antibody No. | HCDR1 | HCDR2 | HCDR3 |
|---|---|---|---|
| PR002055 | 13 | 42 | 82 |
| PR002056 | 10 | 42 | 83 |
| PR002057 | 14 | 42 | 83 |
| PR002058 | 10 | 43 | 84 |
| PR002059 | 13 | 44 | 82 |
| PR002060 | 15 | 44 | 83 |
| PR002061 | 10 | 44 | 83 |
| PR002062 | 10 | 45 | 83 |
| PR002063 | 10 | 42 | 83 |
| PR002064 | 10 | 45 | 83 |
| PR002065 | 10 | 46 | 85 |
| PR002066 | 10 | 44 | 83 |
| PR002067 | 10 | 44 | 86 |
| PR002068 | 16 | 42 | 82 |
| PR002069 | 10 | 47 | 87 |
| PR002070 | 16 | 44 | 83 |
| PR002071 | 10 | 42 | 83 |
| PR002072 | 10 | 45 | 88 |
| PR002073 | 10 | 43 | 84 |
| PR002074 | 10 | 48 | 89 |
| PR002075 | 10 | 49 | 90 |
| PR002076 | 10 | 49 | 83 |
| PR002077 | 10 | 50 | 90 |
| PR005362 | 20 | 44 | 86 |
| PR005363 | 21 | 44 | 86 |
| PR005364 | 10 | 54 | 86 |
| PR005365 | 10 | 55 | 86 |
| PR005366 | 10 | 56 | 86 |
| PR005367 | 10 | 42 | 86 |
| PR005368 | 10 | 57 | 86 |
| PR005369 | 10 | 58 | 86 |
| PR005370 | 10 | 59 | 86 |
| PR005371 | 10 | 60 | 86 |
| PR005372 | 10 | 44 | 94 |
| PR005373 | 10 | 44 | 95 |
| PR005374 | 10 | 44 | 96 |
| PR005375 | 10 | 44 | 97 |
| PR005376 | 10 | 44 | 98 |
| PR005377 | 10 | 44 | 99 |
| PR005378 | 10 | 54 | 95 |
| PR005379 | 10 | 54 | 97 |
| PR005380 | 10 | 55 | 95 |
| PR005381 | 10 | 55 | 97 |
| PR005382 | 10 | 56 | 95 |
| PR005383 | 10 | 56 | 97 |
| PR005384 | 22 | 54 | 86 |
| PR005385 | 23 | 54 | 86 |
| PR005386 | 24 | 54 | 86 |
| PR005387 | 21 | 54 | 86 |
| PR005388 | 10 | 54 | 99 |
| PR005389 | 22 | 54 | 99 |
| PR005390 | 23 | 54 | 99 |
| PR005391 | 24 | 54 | 99 |
| PR005392 | 21 | 54 | 99 |

Preferably, the OX40-targeted antibody or the antigen-binding fragment thereof further comprises a heavy chain constant region Fc domain of a human antibody, wherein the heavy chain constant region Fc domain of the human antibody includes, for example, a heavy chain constant region Fc domain of human IgG1, IgG2, IgG3, or IgG4.

Preferably, the OX40-targeted antibody or the antigen-binding fragment thereof comprises one polypeptide chain, wherein the polypeptide chain comprises an amino acid sequence as set forth in any one of SEQ ID NOs: 208-230 or SEQ ID NOs: 234-264 or a mutation thereof. The mutation is a deletion, substitution or addition of one or more amino acid residues on the amino acid sequence, and the amino acid sequence with the mutation has at least 85% sequence identity to the amino acid sequence and maintains or improves the binding of the antibody to OX40, wherein the at least 85% sequence identity is preferably at least 90% sequence identity, more preferably at least 95% sequence identity, and most preferably at least 99% sequence identity.

Preferably, the OX40-targeted antibody or the antigen-binding fragment thereof includes IgG, Fab, Fab′, F(ab′)₂, Fv, scFv, HCAb, VH, bispecific antibodies, multispecific antibodies, single-domain antibodies, or any other antibodies retaining the ability of an antibody to specifically bind to an antigen (which may be part of the ability of the antibody to specifically bind to the antigen), or monoclonal or polyclonal antibodies prepared from the above antibodies.

In a certain preferred embodiment, the OX40-targeted antibody or the antigen-binding fragment thereof is a blocking antibody.

In a certain preferred embodiment, the OX40-targeted antibody or the antigen-binding fragment thereof is a weakly blocking or non-blocking antibody.

In the present application, a “Fab fragment” consists of one light chain and CH1 and the variable region of one heavy chain. The heavy chain of a Fab molecule cannot form disulfide bonds with another heavy chain molecule. An “Fc” region contains two heavy chain fragments comprising CH2 and CH3 domains of an antibody. The two heavy chain fragments are held together by two or more disulfide bonds and the hydrophobic interaction of the CH3 domains. The “Fab′ fragment” contains one light chain and part of one heavy chain comprising the VH domain and the CH1 domain and the region between the CH1 and CH2 domains, so that interchain disulfide bonds can be formed between the two heavy chains of two Fab′ fragments to provide an F(ab′)₂ molecule. The “F(ab′)₂ fragment” contains two light chains and two heavy chains comprising part of the constant region between the CH1 and CH2 domains, so that interchain disulfide bonds are formed between the two heavy chains. Thus, an F(ab′)₂ fragment consists of two Fab′ fragments held together by disulfide bonds between the two heavy chains. The term “Fv” refers to an antibody fragment consisting of the VL and VH domains of a single arm of an antibody, but lacks the constant region.

In the present application, the scFv (single chain antibody fragment) may be a conventional single chain antibody in the art, which comprises a heavy chain variable region, a light chain variable region, and a short peptide of 15-20 amino acids. In the scFv, the VL and VH domains are paired to form a monovalent molecule via a linker that enables them to produce a single polypeptide chain [see, e.g., Bird et al, Science 242:423-426 (1988) and Huston et al, Proc. Natl. Acad. Sci. USA 85:5879-5883 (1988)]. Such scFv molecules may have a general structure: NH2-VL-linker-VH-COOH or NH2-VH-linker-VL-COOH. An appropriate linker in the prior art consists of repeated G4S amino acid sequences or a variant thereof. For example, linkers having the amino acid sequence (G₄S)₄ or (G₄S)₃ may be used, but a variant thereof may also be used.

In the present application, the term “multispecific antibody” is used in its broadest sense to encompass antibodies having multi-epitope specificity. Those multispecific antibodies include, but are not limited to: an antibody comprising a heavy chain variable region (VH) and a light chain variable region (VL), the VH-VL unit having multi-epitope specificity; an antibody having two or more VL and VH regions, each of the VH-VL units binding to a different target or a different epitope on a same target; an antibody having two or more single variable regions, each of the single variable regions binding to a different target or a different epitope on a same target; full-length antibodies, antibody fragments, bispecific antibodies (diabodies), triabodies, antibody fragments linked together covalently or non-covalently, and the like.

In the present application, the monoclonal antibody or mAb or Ab refers to an antibody obtained from a single clonal cell strain, which is not limited to eukaryotic, prokaryotic, or phage clonal cell strains.

In the present application, the single-domain antibody may be one conventional in the art, which comprises a heavy chain variable region and a heavy chain constant region.

In order to solve the above technical problems, a second aspect of the present invention provides a bispecific binding protein comprising at least two protein functional regions: a protein functional region A and a protein functional region B, wherein the protein functional region A and the protein functional region B target different antigens, wherein the protein functional region B targets OX40 and the protein functional region A targets a non-OX40 antigen; the protein functional region B is selected from the OX40-targeted antibody or the antigen-binding fragment thereof according to the first aspect of the present invention.

Preferably, the protein functional region A targets PD-L1, B7H4, PSMA, EPCAM or CLDN18.2.

Preferably, the protein functional region A is a PSMA antibody or an antigen-binding fragment thereof, an EPCAM antibody or an antigen-binding fragment thereof, a CLDN18.2 antibody or an antigen-binding fragment thereof, a B7H4 antibody or an antigen-binding fragment thereof, or a PD-L 1 antibody or an antigen-binding fragment thereof.

In a certain preferred embodiment, the PD-L1 antibody or the antigen-binding fragment thereof comprises a light chain variable region (VL) and a heavy chain variable region (VH), wherein the VL comprises VL CDR1, VL CDR2 and VL CDR3 with amino acid sequences as set forth in SEQ ID NOs: 111, 119 and 129, respectively, and the VH comprises VH CDR1, VH CDR2 and VH CDR3 with amino acid sequences as set forth in SEQ ID NOs: 10, 39 and 79, respectively.

In a certain preferred embodiment, the EPCAM antibody or the antigen-binding fragment thereof comprises a light chain variable region (VL) and a heavy chain variable region (VH), wherein the VL comprises VL CDR1, VL CDR2 and VL CDR3 with amino acid sequences as set forth in SEQ ID NOs: 112, 120 and 130, respectively, and the VH comprises VH CDR1, VH CDR2 and VH CDR3 with amino acid sequences as set forth in SEQ ID NOs: 11, 40 and 80, respectively.

In a certain preferred embodiment, the PSMA antibody or the antigen-binding fragment thereof comprises a light chain variable region (VL) and a heavy chain variable region (VH), wherein the VL comprises VL CDR1, VL CDR2 and VL CDR3 with amino acid sequences as set forth in SEQ ID NOs: 113, 121 and 131, respectively, and the VH comprises VH CDR1, VH CDR2 and VH CDR3 with amino acid sequences as set forth in SEQ ID NOs: 12, 41 and 81, respectively.

In a certain preferred embodiment, the B7H4 antibody or the antigen-binding fragment thereof comprises a light chain variable region (VL) and a heavy chain variable region (VH), wherein the VL comprises VL CDR1, VL CDR2 and VL CDR3 with amino acid sequences as set forth in SEQ ID NOs: 114, 122 and 132, respectively, and the VH comprises VH CDR1, VH CDR2 and VH CDR3 with amino acid sequences as set forth in SEQ ID NOs: 17, 51 and 91, respectively.

In a certain preferred embodiment, the CLDN18.2 antibody or the antigen-binding fragment thereof comprises a light chain variable region (VL) and a heavy chain variable region (VH), wherein the VL comprises VL CDR1, VL CDR2 and VL CDR3 with amino acid sequences as set forth in SEQ ID NOs: 112, 120 and 133, respectively, and the VH comprises VH CDR1, VH CDR2 and VH CDR3 with amino acid sequences as set forth in SEQ ID NOs: 18, 52 and 92, respectively.

In a preferred embodiment, the bispecific binding protein comprises at least two protein functional regions: a protein functional region A and a protein functional region B, wherein the protein functional region A comprises a light chain variable region (VL) and a heavy chain variable region (VH), wherein the VL comprises VL CDR1, VL CDR2 and VL CDR3 with amino acid sequences as set forth in SEQ ID NOs: 111, 119 and 129, respectively, and the VH comprises VH CDR1, VH CDR2 and VH CDR3 with amino acid sequences as set forth in SEQ ID NOs: 10, 39 and 79, respectively; and the protein functional region B comprises a heavy chain variable region (VH), wherein the VH comprises VH CDR1, VH CDR2 and VH CDR3 with amino acid sequences as set forth in SEQ ID NOs: 10, 44 and 86, respectively.

In a preferred embodiment, the bispecific binding protein comprises at least two protein functional regions: a protein functional region A and a protein functional region B, wherein the protein functional region A comprises a light chain variable region (VL) and a heavy chain variable region (VH), wherein the VL comprises VL CDR1, VL CDR2 and VL CDR3 with amino acid sequences as set forth in SEQ ID NOs: 114, 122 and 132, respectively, and the VH comprises VH CDR1, VH CDR2 and VH CDR3 with amino acid sequences as set forth in SEQ ID NOs: 17, 51 and 91, respectively; and the protein functional region B comprises a heavy chain variable region (VH), wherein the VH comprises VH CDR1, VH CDR2 and VH CDR3 with amino acid sequences as set forth in SEQ ID NOs: 10, 44 and 86, respectively.

In a certain preferred embodiment, the bispecific binding protein comprises at least two protein functional regions: a protein functional region A and a protein functional region B, wherein the protein functional region A comprises a light chain variable region (VL) and a heavy chain variable region (VH), wherein the VL comprises VL CDR1, VL CDR2 and VL CDR3 with amino acid sequences as set forth in SEQ ID NOs: 112, 120 and 130, respectively, and the VH comprises VH CDR1, VH CDR2 and VH CDR3 with amino acid sequences as set forth in SEQ ID NOs: 11, 40 and 80, respectively; and the protein functional region B comprises a heavy chain variable region (VH), wherein the VH comprises VH CDR1, VH CDR2 and VH CDR3 with amino acid sequences as set forth in SEQ ID NOs: 10, 44 and 86, respectively.

In a certain preferred embodiment, the bispecific binding protein comprises at least two protein functional regions: a protein functional region A and a protein functional region B, wherein the protein functional region A comprises a light chain variable region (VL) and a heavy chain variable region (VH), wherein the VL comprises VL CDR1, VL CDR2 and VL CDR3 with amino acid sequences as set forth in SEQ ID NOs: 113, 121 and 131, respectively, and the VH comprises VH CDR1, VH CDR2 and VH CDR3 with amino acid sequences as set forth in SEQ ID NOs: 12, 41 and 81, respectively; and the protein functional region B comprises a heavy chain variable region (VH), wherein the VH comprises VH CDR1, VH CDR2 and VH CDR3 with amino acid sequences as set forth in SEQ ID NOs: 10, 44 and 86, respectively.

In a certain preferred embodiment, the bispecific binding protein comprises at least two protein functional regions: a protein functional region A and a protein functional region B, wherein the protein functional region A comprises a light chain variable region (VL) and a heavy chain variable region (VH), wherein the VL comprises VL CDR1, VL CDR2 and VL CDR3 with amino acid sequences as set forth in SEQ ID NOs: 112, 120 and 133, respectively, and the VH comprises VH CDR1, VH CDR2 and VH CDR3 with amino acid sequences as set forth in SEQ ID NOs: 18, 52 and 92, respectively; and the protein functional region B comprises a heavy chain variable region (VH), wherein the VH comprises VH CDR1, VH CDR2 and VH CDR3 with amino acid sequences as set forth in SEQ ID NOs: 10, 44 and 86, respectively.

In a preferred embodiment, the bispecific binding protein comprises at least two protein functional regions: a protein functional region A and a protein functional region B, wherein the protein functional region A comprises a light chain variable region (VL) and a heavy chain variable region (VH), wherein the VL comprises an amino acid sequence as set forth in SEQ ID NO: 199, and the VH comprises an amino acid sequence as set forth in SEQ ID NO: 139; and the protein functional region B comprises a heavy chain variable region, wherein the VH comprises an amino acid sequence as set forth in SEQ ID NO: 154.

In a preferred embodiment, the bispecific binding protein comprises at least two protein functional regions: a protein functional region A and a protein functional region B, wherein the protein functional region A comprises a light chain variable region (VL) and a heavy chain variable region (VH), wherein the VL comprises an amino acid sequence as set forth in SEQ ID NO: 202, and the VH comprises an amino acid sequence as set forth in SEQ ID NO: 165; and the protein functional region B comprises a heavy chain variable region (VH), wherein the VH comprises an amino acid sequence as set forth in SEQ ID NO: 154.

In a certain preferred embodiment, the bispecific binding protein comprises at least two protein functional regions: a protein functional region A and a protein functional region B, wherein the protein functional region A comprises a light chain variable region (VL) and a heavy chain variable region (VH), wherein the VL comprises an amino acid sequence as set forth in SEQ ID NO: 200, and the VH comprises an amino acid sequence as set forth in SEQ ID NO: 140; and the protein functional region B comprises a heavy chain variable region (VH), wherein the VH comprises an amino acid sequence as set forth in SEQ ID NO: 154.

In a certain preferred embodiment, the bispecific binding protein comprises at least two protein functional regions: a protein functional region A and a protein functional region B, wherein the protein functional region A comprises a light chain variable region (VL) and a heavy chain variable region (VH), wherein the VL comprises an amino acid sequence as set forth in SEQ ID NO: 201, and the VH comprises an amino acid sequence as set forth in SEQ ID NO: 141; and the protein functional region B comprises a heavy chain variable region (VH), wherein the VH comprises an amino acid sequence as set forth in SEQ ID NO: 154.

In a certain preferred embodiment, the bispecific binding protein comprises at least two protein functional regions: a protein functional region A and a protein functional region B, wherein the protein functional region A comprises a light chain variable region (VL) and a heavy chain variable region (VH), wherein the VL comprises an amino acid sequence as set forth in SEQ ID NO: 203, and the VH comprises an amino acid sequence as set forth in SEQ ID NO: 166; and the protein functional region B comprises a heavy chain variable region (VH), wherein the VH comprises an amino acid sequence as set forth in SEQ ID NO: 154.

Preferably, the protein functional region A and/or the protein functional region B is in the form of IgG, Fab′, F(ab′)₂, Fv, scFv, VH or HCAb, wherein the protein functional region A and the protein functional region B are not both IgG.

More preferably, the heavy chain constant region of IgG is a human heavy chain constant region, more preferably a human IgG1, human IgG2, human IgG3, or human IgG4 heavy chain constant region, wherein the human IgG preferably comprises one, two or three mutations of L234A, L235A and P329G, and more preferably comprises mutations of L234A and L235A or mutations of L234A, L235A and P329G.

More preferably, the number of the Fab, Fab′, F(ab′)2, Fv, scFv or VH is one or more than one.

Preferably, the protein functional region B is of a single VH structure, and the protein functional region A is of an IgG structure; and the protein functional region B is preferably linked to the C-terminus of the protein functional region A.

In a preferred embodiment, the bispecific antibody comprises a first polypeptide chain and a second polypeptide chain, wherein the first polypeptide chain is as shown in formula: N′-VL_{_A}-CL-C′, and the second polypeptide chain is as shown in formula: N′-VH_{_A}-CH1-h-CH2-CH3-L-VH_{_B}-C′;

wherein the VH_{_B} is VH of the protein functional region B, the VL_{_A} and the VH_{_A} are VL and VH of the protein functional region A, respectively, the h is a hinge region, and the L is a linker peptide;
wherein the hinge region is conventional in the art, generally contains a large amount of proline, and has elasticity.

Preferably, the L is preferably 0 in length or has an amino acid sequence as set forth in any one of SEQ ID NOs: 278-295. In one embodiment, CH3 is fusion-linked directly to VH_{_B} in the second polypeptide chain, i.e., L is 0 in length. In another embodiment, CH3 is linked to VH_{_B} via a linker peptide L in the second polypeptide chain; L may be the sequence listed in Table 11 in the examples.

Preferably, the protein functional region B is of an HCAb structure, and the protein functional region A is of a Fab structure; and the protein functional region B is preferably linked to the C-terminus of the protein functional region A.

In a preferred embodiment, the bispecific antibody comprises a first polypeptide chain and a second polypeptide chain, wherein the first polypeptide chain is as shown in formula: N′-VH_{_A}-CH1-C′, and the second polypeptide chain is as shown in formula: N′-VL_{_A}-CL-L1-VH_{_B}-L2-CH2-CH3-C′.

In a preferred embodiment, the bispecific antibody comprises a first polypeptide chain and a second polypeptide chain, wherein the first polypeptide chain is as shown in formula: N′-VL_{_A}-CL-C′, and the second polypeptide chain is as shown in formula: N′-VH_{_A}-CH1-L1-VH_{_B}-L2-CH2-CH3-C′;

wherein the VH_{_B} is VH of the protein functional region B, the VL_{_A} and the VH_{_A} are VL and VH of the protein functional region A, respectively, and the L1 and L2 are linker peptides;
wherein the L1 or L2 is preferably 0 in length or preferably has an amino acid sequence as set forth in any one of SEQ ID NOs: 278-295 or an amino acid sequence of GS, for example, the L1 has an amino acid sequence as set forth in SEQ ID NO: 286, and the L2 has an amino acid sequence as set forth in SEQ ID NO: 285; wherein VH_{_B} of is linked to CH2 via a linker peptide L2 in the second polypeptide chain; L2 may be a hinge region or a hinge region-derived linker peptide sequence or the sequence listed in Table 11, preferably the sequence of human IgG1 hinge, human IgG1 hinge (C220S) or G5-LH. In one embodiment, CL is fusion-linked directly to VH_{_B} in the second polypeptide chain, i.e., L1 is 0 in length. In another embodiment, CL is linked to VH_{_B} via a linker peptide L1 in the second polypeptide chain; and L1 may be the sequence listed in Table 11 in the examples.

In a certain preferred embodiment, the bispecific binding protein comprises a first polypeptide chain and a second polypeptide chain, wherein the first polypeptide chain comprises an amino acid sequence as set forth in SEQ ID NO: 265, and the second polypeptide chain comprises an amino acid sequence as set forth in SEQ ID NO: 271.

In a certain preferred embodiment, the bispecific binding protein comprises a first polypeptide chain and a second polypeptide chain, wherein the first polypeptide chain comprises an amino acid sequence as set forth in SEQ ID NO: 268, and the second polypeptide chain comprises an amino acid sequence as set forth in SEQ ID NO: 272.

In a certain preferred embodiment, the bispecific binding protein comprises a first polypeptide chain and a second polypeptide chain, wherein the first polypeptide chain comprises an amino acid sequence as set forth in SEQ ID NO: 273, and the second polypeptide chain comprises an amino acid sequence as set forth in SEQ ID NO: 274.

In a certain preferred embodiment, the bispecific binding protein comprises a first polypeptide chain and a second polypeptide chain, wherein the first polypeptide chain comprises an amino acid sequence as set forth in SEQ ID NO: 266, and the second polypeptide chain comprises an amino acid sequence as set forth in SEQ ID NO: 275.

In a certain preferred embodiment, the bispecific binding protein comprises a first polypeptide chain and a second polypeptide chain, wherein the first polypeptide chain comprises an amino acid sequence as set forth in SEQ ID NO: 267, and the second polypeptide chain comprises an amino acid sequence as set forth in SEQ ID NO: 276.

In a certain preferred embodiment, the bispecific binding protein comprises a first polypeptide chain and a second polypeptide chain, wherein the first polypeptide chain comprises an amino acid sequence as set forth in SEQ ID NO: 269, and the second polypeptide chain comprises an amino acid sequence as set forth in SEQ ID NO: 277.

In order to solve the above technical problems, a third aspect of the present invention provides a chimeric antigen receptor comprising the OX40-targeted antibody or the antigen-binding fragment thereof according to the first aspect of the present invention or the bispecific antibody according to the second aspect of the present invention.

In order to solve the above technical problems, a fourth aspect of the present invention provides an immune cell comprising the chimeric antigen receptor according to the third aspect of the present invention. Preferably, the immune cell is a T cell or an NK cell.

In order to solve the above technical problems, a fifth aspect of the present invention provides an isolated nucleic acid encoding the OX40-targeted antibody or the antigen-binding fragment thereof according to the first aspect of the present invention, or the bispecific antibody according to the second aspect of the present invention, or the chimeric antigen receptor according to the third aspect of the present invention.

The preparation method for the nucleic acid is a conventional preparation method in the art, and preferably comprises the following steps: obtaining a nucleic acid molecule encoding the above antibody by gene cloning technology, or obtaining a nucleic acid molecule encoding the above antibody by artificial complete sequence synthesis.

It is known to those skilled in the art that substitutions, deletions, alterations, insertions or additions may be appropriately introduced into the base sequence encoding the amino acid sequence of the above antibody to provide a polynucleotide homologue. The polynucleotide homologue of the present invention may be produced by substituting, deleting or adding one or more bases of a gene encoding the antibody sequence within a range in which the activity of the antibody is maintained.

In order to solve the above technical problems, a sixth aspect of the present invention provides a recombinant expression vector comprising the isolated nucleic acid according to the fifth aspect of the present invention.

The recombinant expression vector may be obtained by using conventional methods in the art, i.e., by linking the nucleic acid molecule of the present application to various expression vectors. The expression vector is any conventional vector in the art, provided that it can carry the aforementioned nucleic acid molecule.

Preferably, the expression vector comprises a eukaryotic cell expression vector and/or a prokaryotic cell expression vector.

In order to solve the above technical problems, a seventh aspect of the present invention provides a transformant comprising the isolated nucleic acid according to the fifth aspect of the present invention or the recombinant expression vector according to the sixth aspect of the present invention.

The transformant may be prepared by using conventional methods in the art, e.g., by transforming the above recombinant expression vector into a host cell. The host cell of the transformant is any conventional host cell in the art, provided that it can enable the stable replication of the above recombinant expression vector and the nucleic acid carried can be efficiently expressed. Preferably, the host cell is a prokaryotic and/or a eukaryotic cell, wherein the prokaryotic cell is preferably an E. coli cell such as TG1 and BL21 (expressing a single chain antibody or Fab antibody), and the eukaryotic cell is preferably an HEK293 cell or a CHO cell (expressing a full-length IgG antibody). The preferred recombinant expression transformant of the present invention can be obtained by transforming the aforementioned recombinant expression plasmid into a host cell. The transformation method is a conventional transformation method in the art, preferably a chemical transformation method, a heat shock method or an electric transformation method.

In order to solve the above technical problems, an eighth aspect of the present invention provides a method for preparing an OX40-targeted antibody or an antigen-binding fragment thereof, or a bispecific antibody, which comprises culturing the transformant according to the seventh aspect of the present invention, and obtaining the OX40-targeted antibody or the antigen-binding fragment thereof, or the bispecific antibody from a culture.

In order to solve the above technical problems, a ninth aspect of the present invention provides an antibody-drug conjugate comprising an antibody moiety and a conjugate moiety, wherein the antibody moiety comprises the OX40-targeted antibody or the antigen-binding fragment thereof according to the first aspect of the present invention and/or the bispecific antibody according to the second aspect of the present invention, and the conjugate moiety includes, but is not limited to, a detectable label, a drug, a toxin, a cytokine, a radionuclide, an enzyme, or a combination thereof; the antibody moiety and the conjugate moiety are conjugated via a chemical bond or a linker.

In order to solve the above technical problems, a tenth aspect of the present invention provides a pharmaceutical composition comprising the OX40-targeted antibody or the antigen-binding fragment thereof according to the first aspect of the present invention, the bispecific antibody according to the second aspect of the present invention, and a pharmaceutically acceptable carrier.

Preferably, the pharmaceutical composition further comprises an additional anti-tumor antibody as an active ingredient.

The pharmaceutically acceptable carrier may be a carrier conventional in the art, and the carrier may be any suitable physiologically or pharmaceutically acceptable auxiliary material. The pharmaceutically acceptable auxiliary material is one conventional in the art, and preferably comprises a pharmaceutically acceptable excipient, a filler, a diluent, or the like. More preferably, the pharmaceutical composition comprises 0.01%-99.99% of the protein and/or the antibody-drug conjugate and 0.01%-99.99% of a pharmaceutically acceptable carrier, the percentage being the mass percentage of the pharmaceutical composition.

The route of administration for the pharmaceutical composition of the present invention is preferably parenteral administration, injection administration or oral administration. The injection administration preferably includes intravenous injection, intramuscular injection, intraperitoneal injection, intradermal injection or subcutaneous injection. The pharmaceutical composition is in any conventional dosage form in the art, preferably in the form of a solid, semisolid or liquid, i.e., it may be an aqueous solution, a non-aqueous solution or a suspension, more preferably a tablet, capsule, granule, injection, infusion, or the like. More preferably, it is administered intravascularly, subcutaneously, intraperitoneally or intramuscularly. Preferably, the pharmaceutical composition may also be administered as an aerosol or a coarse spray, i.e., administered nasally; or administered intrathecally, intramedullarily or intraventricularly. More preferably, the pharmaceutical composition may also be administered transdermally, percutaneously, topically, enterally, intravaginally, sublingually or rectally. The pharmaceutical composition of the present invention may be formulated into various dosage forms as required, and can be administered by a physician in the light of the patient's type, age, weight, and general disease state, route of administration, etc. The administration may be performed, for example, by injection or other therapeutic modalities.

The dosage level at which the pharmaceutical composition of the present invention is administered can be adjusted depending on the amount of the composition to achieve the desired diagnostic or therapeutic outcome. The dosage regimen may also be a single injection or multiple injections, or an adjusted one. The selected dosage level and regimen is appropriately adjusted depending on a variety of factors including the activity and stability (i.e., half-life) of the pharmaceutical composition, the formulation, the route of administration, combination with other drugs or treatments, the disease or disorder to be detected and/or treated, and the health condition and past medical history of the subject to be treated.

A therapeutically effective dose for the pharmaceutical composition of the present invention may be estimated initially in cell culture experiments or animal models such as rodents, rabbits, dogs, pigs and/or primates. Animal models can also be used to determine the appropriate concentration range and route of administration, and subsequently an effective dose and a route of administration in humans. In general, the determination of and adjustment to the effective amount or dose to be administered and the assessment of when and how to make such adjustments are known to those skilled in the art.

For combination therapy, the above OX40-targeted antibody, the above antibody-drug conjugate and/or an additional therapeutic or diagnostic agent may each be used as a single agent for use within any time frame suitable for performing the intended treatment or diagnosis. Thus, these single agents may be administered substantially simultaneously (i.e., as a single formulation or within minutes or hours) or sequentially.

For additional guidance regarding formulations, doses, dosage regimens, and measurable therapeutic outcomes, see Berkow et al. (2000) The Merck Manual of Medical Information and Merck & Co. Inc., Whitehouse Station, New Jersey; Ebadi (1998) CRC Desk Reference of Clinical Pharmacology, etc.

In order to solve the above technical problems, an eleventh aspect of the present invention provides use of the OX40-targeted antibody or the antigen-binding fragment thereof according to the first aspect of the present invention, the bispecific antibody according to the second aspect of the present invention, the chimeric antigen receptor according to the third aspect of the present invention, the immune cell according to the fourth aspect of the present invention, the antibody-drug conjugate according to the ninth aspect of the present invention, and/or the pharmaceutical composition according to the tenth aspect of the present invention in the preparation of a medicament, a kit, and/or an administration device for the diagnosis, prevention and/or treatment of a tumor; or provides the OX40-targeted antibody or the antigen-binding fragment according to the first aspect of the present invention, the bispecific antibody according to the second aspect of the present invention, the chimeric antigen receptor according to the third aspect of the present invention, the immune cell according to the fourth aspect of the present invention, the antibody-drug conjugate according to the ninth aspect of the present invention and/or the pharmaceutical composition according to the tenth aspect of the present invention, for use in the diagnosis, prevention and/or treatment of a tumor.

Preferably, when the bispecific antibody is used for the preparation of a medicament for the diagnosis, prevention and/or treatment of a tumor, the tumor is a PSMA, EPCAM, CLDN18.2, B7H4 and/or PD-L1-associated positive tumor, such as breast cancer, pancreatic cancer, gastric cancer and/or prostate cancer, or a metastatic lesion thereof.

In order to solve the above technical problems, a twelfth aspect of the present invention provides a method for detecting OX40 in a sample, which comprises detecting with the OX40-targeted antibody or the antigen-binding fragment thereof according to the first aspect of the present invention and/or the bispecific antibody according to the second aspect of the present invention.

Preferably, the method is for non-diagnostic purposes.

In order to solve the above technical problems, a thirteenth aspect of the present invention provides a kit comprising the OX40-targeted antibody or the antigen-binding fragment thereof according to the first aspect of the present invention, the bispecific antibody according to the second aspect of the present invention, the chimeric antigen receptor according to the third aspect of the present invention, the immune cell according to the fourth aspect of the present invention, the antibody-drug conjugate according to the ninth aspect of the present invention, and/or the pharmaceutical composition according to the tenth aspect of the present invention, and optionally instructions.

In order to solve the above technical problems, a fourteenth aspect of the present invention provides an administration device comprising: (1) an infusion module for administering to a subject in need thereof the pharmaceutical composition according to the tenth aspect of the present invention, and (2) optionally a pharmacodynamic monitoring module.

In order to solve the above technical problems, the present invention also provides use of the OX40-targeted antibody or the antigen-binding fragment thereof according to the first aspect of the present invention, the bispecific antibody according to the second aspect of the present invention, the chimeric antigen receptor according to the third aspect of the present invention, the immune cell according to the fourth aspect of the present invention, the antibody-drug conjugate according to the ninth aspect of the present invention, and/or the pharmaceutical composition according to the tenth aspect of the present invention in the diagnosis, prevention and/or treatment of a tumor. Preferably, the tumor is one according the eleventh aspect of the present invention.

In order to solve the above technical problems, the present invention also provides a kit of parts comprising a kit A and a kit B, wherein the kit A is the OX40-targeted antibody or the antigen-binding fragment thereof according to the first aspect of the present invention, the bispecific antibody according to the second aspect of the present invention, the chimeric antigen receptor according to the third aspect of the present invention, the immune cell according to the fourth aspect of the present invention, the antibody-drug conjugate according to the ninth aspect of the present invention, or the pharmaceutical composition according to the tenth aspect of the present invention; and the kit B is an additional anti-tumor antibody or a pharmaceutical composition comprising the additional anti-tumor antibody. The kit A and the kit B may be used simultaneously, or the kit A may be used prior to the use of the kit B, or the kit B may be used prior to the use of the kit A. The sequence of use can be determined according to actual requirements in a specific application.

In order to solve the above technical problems, the present invention also provides a method for diagnosing, preventing and/or treating a tumor, which comprises administering to a subject in need thereof a therapeutically effective amount of the OX40-targeted antibody or the antigen-binding fragment thereof according to the first aspect of the present invention, the bispecific antibody according to the second aspect of the present invention, the chimeric antigen receptor according to the third aspect of the present invention, the immune cell according to the fourth aspect of the present invention, the antibody-drug conjugate according to the ninth aspect of the present invention, and/or the pharmaceutical composition according to the tenth aspect of the present invention.

In the present application, unless otherwise defined, the scientific and technical terms used herein have the meanings generally understood by those skilled in the art. In addition, the laboratory operations of cell culture, molecular genetics, nucleic acid chemistry and immunology used herein are the routine procedures widely used in the corresponding fields. Meanwhile, in order to better understand the present invention, the definitions and explanations of the relevant terms are provided below.

In the present application, the term “variable” generally refers to the fact that certain portions of the sequences of the variable domains of antibodies vary considerably, resulting in the binding and specificity of various particular antibodies to their particular antigens. However, variability is not evenly distributed throughout the variable region of the antibody. It is concentrated in three segments in each of the light chain and heavy chain variable regions called complementarity determining regions (CDRs) or hypervariable regions (HVRs). The more highly conserved portions of the variable domains are called frameworks (FWRs). The variable domains of native heavy and light chains each comprise four FWRs largely in a β-sheet configuration. The FRs are connected by three CDRs to form a loop connection, and in some cases to form part of a β-sheet structure. The CDRs in each chain are held in close proximity by the FWRs and form, together with the CDRs from the other chain, antigen-binding sites of the antibody. The constant regions are not directly involved in the binding of the antibody to antigens, but they exhibit different effector functions, for example, being involved in antibody-dependent cytotoxicity of the antibody.

The three-letter codes and single-letter codes for amino acids used in the present application are known to those skilled in the art, or are described in J. Biol. Chem, 243, p3558 (1968).

As used herein, the term “include/includes/including” or “comprise/comprises/comprising” is intended to mean that a composition and a method include the elements described but does not exclude other elements; but the case of “consist/consists/consisting of” is also included as the context dictates.

In the present application, the HCAb may be a full human antibody (heavy chain only antibody) produced from a transgenic mouse carrying an immune repertoire of human immunoglobulins-Harbour HCAb mouse (Harbour Antibodies BV, WO 2002/085945 A3) and containing only “heavy chains”, which is only half the size of conventional IgG antibodies and generally have only human antibody “heavy chain” variable domains and mouse Fc constant domains.

The term “antibody” described herein may include an immunoglobulin, which is of a tetrapeptide chain structure formed by connection between two identical heavy chains and two identical light chains by interchain disulfide bonds. Immunoglobulins differ in amino acid composition and arrangement of their heavy chain constant regions and therefore in their antigenicity. Accordingly, immunoglobulins can be classified into five classes, or isotypes of immunoglobulins, namely IgM, IgD, IgG, IgA and IgE, with their corresponding heavy chains being the μ, δ, γ, α and ε chains, respectively. The Ig of the same class can be divided into different subclasses according to the differences in amino acid composition of the hinge regions and the number and location of disulfide bonds in the heavy chains; for example, IgG can be divided into IgG1, IgG2, IgG3, and IgG4. Light chains are classified into κ or λ chains by the difference in the constant regions. Each of the five classes of Ig can have a κ chain or a λ chain.

In the present application, the light chain variable region of the antibody of the present application may further comprise a light chain constant region comprising a human κ or λ chain or a variant thereof. In the present application, the heavy chain variable region of the antibody of the present application may further comprise a heavy chain constant region comprising human IgG1, IgG2, IgG3, IgG4 or a variant thereof.

In light chains and heavy chains, the variable region and constant region are linked by a “J” region of about 12 or more amino acids, and the heavy chain further comprises a “D” region of about 3 or more amino acids. Each heavy chain consists of a heavy chain variable region (VH) and a heavy chain constant region (CH). The heavy chain constant region consists of 3 domains (CH1, CH2 and CH3). Each light chain consists of a light chain variable region (VL) and a light chain constant region (CL). The light chain constant region consists of one domain CL. The constant region of the antibody can mediate the binding of immunoglobulins to host tissues or factors, including the binding of various cells of the immune system (e.g., effector cells) to the first component (C1q) of classical complement system. The sequences of about 110 amino acids of the heavy and light chains of the antibody near the N-terminus vary considerably and thus are referred to as variable regions (V regions); the remaining amino acid sequences near the C-terminus are relatively stable and thus are referred to as constant regions (C regions). The variable regions comprise 3 hypervariable regions (HVRs) and 4 framework regions (FWRs) with relatively conservative sequences. The 3 hypervariable regions determine the specificity of the antibody and thus are also known as complementarity determining regions (CDRs). Each of the light chain variable regions (VLs) and the heavy chain variable region (VHs) consists of 3 CDR regions and 4 FWR regions arranged from the amino-terminus to the carboxyl-terminus in the following order: FWR1, CDR1, FWR2, CDR2, FWR3, CDR3, and FWR4. The 3 CDR regions of the light chain refer to VL CDR1, VL CDR2 and VL CDR3; and the 3 CDR regions of the heavy chain refer to VH CDR1, VH CDR2 and VH CDR3.

The term “human antibody” includes antibodies having variable and constant regions of human germline immunoglobulin sequences. The human antibody of the present application may include amino acid residues not encoded by human germline immunoglobulin sequences (e.g., mutations introduced by in vitro random or site-directed mutagenesis or in vivo somatic mutation). However, the term “human antibody” does not include antibodies in which CDR sequences derived from the germline of another mammalian species, such as a mouse, have been grafted into human framework sequences (i.e., “humanized antibodies”).

As used herein, the term “specificity” with respect to an antibody means that an antibody recognizes a specific antigen but does not substantially recognize or bind to other molecules in a sample. For example, an antibody specifically binding to an antigen from one species may also bind to the antigen from one or more species. However, such interspecific cross-reactivity per se does not change the classification of antibodies by specificity. In another example, an antibody specifically binding to an antigen may also bind to the antigen in different allelic forms. However, such cross-reactivity per se does not change the classification of antibodies by specificity. In some cases, the term “specificity” or “specifically binding” may be used to refer to the interaction of an antibody, a protein or a peptide with a second chemical substance, meaning that the interaction is dependent on the presence of a particular structure (e.g., an antigenic determinant or epitope) in the chemical substance; for example, an antibody generally recognizes and binds to a particular protein structure rather than a protein. If an antibody has specificity to an epitope “A”, then in a reaction containing labeled “A” and the antibody, the presence of a molecule containing the epitope A (or free, unlabeled A) will reduce the amount of labeled A bound to the antibody.

In the present application, the term “antigen-binding fragment” refers to an antigen-binding fragment and an antibody analog of the antibody, which generally include at least a portion of the antigen-binding region or variable region (e.g., one or more CDRs) of a parental antibody. The antibody fragment retains at least some of the binding specificities of the parental antibody. Generally, the antibody fragment retains at least 10% of the binding activity of the parental antibody when the activity is expressed on a molar basis. Preferably, the antibody fragment retains at least 50%, 60%, 70%, 80%, 90%, 95%, 99%, or 100% or more of the binding affinity of the parental antibody for the target. Examples of the antigen-binding fragment include, but are not limited to: Fab, Fab′, F(ab′)₂, Fv, linear antibodies, single chain antibodies, nanobodies, domain antibodies and multispecific antibodies. Engineered antibody variants are reviewed in Holliger and Hudson (2005) Nat. Biotechnol. 23: 1126-1136.

The term “chimeric antigen receptor” or “CAR” used herein includes extracellular domains (extracellular binding domains), hinge domains, transmembrane domains (transmembrane regions) capable of binding to antigens and polypeptides that causes passes a cytoplasmic signal to a domain (i.e., an intracellular signal domain). The hinge domain may be considered as a part for providing flexibility to an extracellular antigen-binding region. The intracellular signal domain refers to a protein that transmits information into a cell via a determined signaling pathway by generating a second messenger to regulate the activity of the cell, or a protein that functions as an effector by corresponding to such a messenger. It generates a signal that can promote the immune effector function of a cell of the CAR (e.g., a CART cell). The intracellular signal domain includes a signaling domain, and may also include a co-stimulatory intracellular domain derived from a co-stimulatory molecule.

“Identity” or “mutation” refers to sequence similarity between two polynucleotide sequences or between two polypeptide sequences. When positions in two compared sequences are all occupied by the same base or amino acid monomer subunit, for example, if a position in each of two DNA molecules is occupied by adenine, the molecules are homologous at that position. The identity percentage between two sequences is a function of the number of matched or homologous positions shared by the two sequences divided by the number of the compared positions×100%. For example, when sequences are optimally aligned, if 6 out of 10 positions in two sequences match or are homologous, the two sequences are 60% homologous. In general, the comparison is made when two aligned sequences give the greatest identity percentage.

The terms “polypeptide”, “peptide” and “protein” (if single-stranded) are used interchangeably in the present application. The terms “nucleic acid”, “nucleic acid sequence”, “nucleotide sequence” or “polynucleotide sequence”, and “polynucleotide” are used interchangeably.

The term “vector” used herein is a composition that comprises an isolated nucleic acid and is useful for delivering the isolated nucleic acid to the interior of a cell. Many vectors are known in the art. They include, but are not limited to, linear polynucleotides, polynucleotides associated with ionic or amphiphilic compounds, plasmids, and viruses. Thus, the term “vector” includes autonomously replicating plasmids or viruses. The term should also be construed as including non-plasmid and non-viral compounds that facilitate the transfer of nucleic acids into cells, such as polylysine compounds and liposomes. Examples of viral vectors include, but are not limited to, adenoviral vectors, adeno-associated viral vectors, retroviral vectors, etc.

The expressions “cell” and “cell line” used in the present application are used interchangeably and all such designations include progeny. The term “host cell” refers to a cell to which a vector can be introduced, including, but not limited to, prokaryotic cells such as E. coli, fungal cells such as yeast cells, or animal cells such as fibroblasts, CHO cells, COS cells, NSO cells, HeLa cells, BHK cells, HEK 293 cells, or human cells.

The term “transfection” refers to the introduction of an exogenous nucleic acid into a eukaryotic cell. Transfection may be accomplished by a variety of means known in the art, including calcium phosphate-DNA co-precipitation, DEAE-dextran mediated transfection, polybrene-mediated transfection, electroporation, microinjection, liposome fusion, lipofection, protoplast fusion, retroviral infection, and biolistics.

The term “immune cell” refers to a cell that can elicit an immune response. The “immune cell” and other grammatical variations thereof may refer to an immune cell of any origin. The “immune cell” includes, for example, white blood cells (leukocytes) and lymphocytes (T cells, B cells, and natural killer (NK) cells) derived from hematopoietic stem cells (HSCs) produced in the bone marrow, and bone marrow-derived cells (neutrophils, eosinophils, basophils, monocytes, macrophages, dendritic cells). The term “immune cell” may also refer to a human or non-human immune cell. For example, the immune cells may be derived from the blood, such as autologous T cells, allogeneic T cells, autologous NK cells and allogeneic NK cells, or from cell lines, such as NK cell lines prepared by infection with the EBV virus, NK cells obtained by induced differentiation of embryonic stem cells and iPSCs, as well as NK92 cell lines.

As used herein, the term “T cell” refers to a class of lymphocytes that mature in the thymus. T cells play an important role in cell-mediated immunity and are different from other lymphocytes (e.g., B cells) in that T cell receptors are present on the cell surface. The “T cell” includes all types of immune cells expressing CD3, including T helper cells (CD4⁺ cells), cytotoxic T cells (CD8⁺ cells), natural killer T cells, T regulatory cells (Tregs), and γ-δT cells. The “cytotoxic cells” include CD8⁺ T cells, natural killer (NK) cells and neutrophils, which are capable of mediating a cytotoxic response. As used herein, the term “NK cell” refers to a class of lymphocytes that originate in the bone marrow and play an important role in the innate immune system. NK cells provide a rapid immune response against virus-infected cells, tumor cells or other stressed cells, even in the absence of antibodies and major histocompatibility complexes on the cell surface.

The term “optional”, “optionally”, “any” or “any one of” means that the event or circumstance subsequently described may, but not necessarily, occur, and that the description includes instances where the event or circumstance occurs or does not occur. For example, “optionally comprising 1 antibody heavy chain variable region” means that the antibody heavy chain variable region of a particular sequence may, but not necessarily, be present. As used herein, the “a” and “an” are used in the present invention to refer to one or more grammatical objects. Unless otherwise specifically stated in the content, the term “or” is used in the present invention to refer to, and is interchangeable with, the term “and/or”. The “about” and “approximately” shall generally mean an acceptable degree of error in the measured quantity in view of the nature or accuracy of the measurement. Exemplary degrees of error are typically within 10% thereof and more typically within 5% thereof. The method and composition disclosed in the present invention encompass polypeptides and nucleic acids having a specified sequence, a variant sequence, or a sequence substantially identical or similar thereto, e.g., a sequence that is at least 85%, 90%, 95%, 99% or more identical to the sequence specified. In the context of amino acid sequences, the term “substantially identical” is used herein to refer to a first amino acid sequence.

As used herein, the term “pharmaceutically acceptable carrier” refers to a carrier that is pharmacologically and/or physiologically compatible with the subject and the active ingredient, is well known in the art (see, e.g., Remington's Pharmaceutical Sciences. Edited by Gennaro A R, 19th ed. Pennsylvania: Mack Publishing Company, 1995), and includes, but is not limited to: pH adjusting agents, surfactants, adjuvants, ionic strength enhancers, diluents, osmotic pressure-maintaining agents, absorption-retarding agents, and preservatives. For example, pH adjusting agents include, but are not limited to, phosphate buffers. Surfactants include, but are not limited to, cationic, anionic or nonionic surfactants, e.g., Tween-80. Ionic strength enhancers include, but are not limited to, sodium chloride. Preservatives include, but are not limited to, various antibacterial and antifungal agents, such as paraben, chloretone, phenol, and sorbic acid. Osmotic pressure-maintaining agents include, but are not limited to, sugars, NaCl, and the like. Absorption-retarding agents include, but are not limited to, monostearate salts and gelatin. Diluents include, but are not limited to, water, aqueous buffers (e.g., buffered saline), alcohols and polyols (e.g., glycerol), and the like. Preservatives include, but are not limited to, various antibacterial and antifungal agents, such as thiomersalate, 2-phenoxyethanol, paraben, chloretone, phenol, and sorbic acid. Stabilizers have the meaning commonly understood by those skilled in the art, and they are capable of stabilizing the desired activity of the active ingredient in a drug, and include, but are not limited to, sodium glutamate, gelatin, SPGA, sugars (such as sorbitol, mannitol, starch, sucrose, lactose, dextran, or glucose), amino acids (such as glutamic acid or glycine), proteins (such as dried whey, albumin, or casein) or degradation products thereof (such as lactalbumin hydrolysate), and the like.

As used herein, the term “EC₅₀” refers to the concentration for 50% of maximal effect, i.e., the concentration that can cause 50% of the maximal effect.

As used herein, the terms “cancer” and “cancer patient” are intended to include all types of cancerous growths or oncogenic processes, metastatic tissues or malignantly transformed cells, tissues or organs, regardless of their histopathological types or stages of invasiveness. Examples include, but are not limited to, solid tumors, hematologic cancer, soft tissue tumors and metastatic lesions.

On the basis of the general knowledge in the art, the above preferred conditions can be combined arbitrarily to obtain preferred embodiments of the present invention.

The reagents and starting materials used in the present invention are commercially available.

The beneficial effects of the present invention are as follows:
1. The OX40 antibodies of the present invention may be fully human “heavy chain”-only antibodies, which are only half the size of conventional IgG antibodies, and which, due to the absence of light chains, can be used for bispecific antibodies while solving the problems of light chain mismatch and heterodimerization; the fully human antibodies can be safely administered to a human subject without eliciting an immunogenic response.
2. The antibodies or the antigen-binding fragments thereof of the present invention have activity in specifically binding to human OX40 and cynomolgus monkey (cyno) OX40. In addition, the antibodies or the antigen-binding fragments thereof of the present invention can promote greater activation of NF-Kb, thereby stimulating the OX40 signaling pathway, and can activate the OX40 pathway in vitro and induce the activation of T cells, with the activation effect comparable to or greater than existing antibodies (e.g., Pogalizumab). Meanwhile, the antibodies or the antigen-binding fragments thereof of the present invention have CD32b crosslinking dependence. In a certain preferred embodiment of the present invention, the OX40-targeted antibody is capable of binding to human OX40 and cynomolgus monkey OX40 proteins, with the binding ability increasing in a positive correlation with the antibody concentration. In a certain preferred embodiment of the present invention, the antibody or the antigen-binding fragment thereof of the present invention is capable of specifically binding to a cell line overexpressing OX40, but not to other members of the TNF tumor necrosis factor receptor superfamily. In a certain preferred embodiment of the present invention, the antibodies or the antigen-binding fragments thereof of the present invention may be fully human “heavy chain”-only antibodies targeting OX40, which are only half the size of conventional IgG antibodies, and which, due to the absence of light chains, can be used for bispecific antibodies while solving the problems of light chain mismatch and heterodimerization.
3. In the preparation of bispecific antibodies using the antibody or the antigen-binding fragment thereof of the present invention, the bispecific antibodies are all capable of binding to human OX40 and the corresponding tumor-associated antigens, and the bispecific antibodies of the present invention do not affect the binding to tumor cells. In a certain preferred embodiment of the present invention, the bispecific antibody has one or two or three or more sites for binding to OX40, thereby enabling optimization of the activity at the OX40 end. In a certain preferred embodiment of the present invention, one end of the bispecific antibody is capable of recognizing and specifically binding to tumor cells, such as EPCAM, PSMA, CLDN18.2, B7H4 or PD-L1, thereby specifically activating T cells in the tumor microenvironment and reducing toxicity caused by OX40 activation.

### BRIEF DESCRIPTION OF THE DRAWINGS

FIGS. 1A-1C show the results of the binding of anti-OX40 HCAb antibodies to a human OX40 protein.

FIGS. 2A-2C show the results of the binding of anti-OX40 HCAb antibodies to a cynomolgus monkey OX40 protein.

FIGS. 3A-3C show the results of the in vitro binding of anti-OX40 HCAb antibodies to CHO-K1/human OX40 cells.

FIGS. 4A-4C show the results of the blocking of anti-OX40 HCAb antibodies on the binding of a human OX40 ligand to human OX40 on the cell surface.

FIGS. 5A-5F show the results of the stimulation of the OX40 signaling pathway by OX40 antibodies as assayed using a reporter gene cell line.

FIG. 6A shows that most OX40 antigen-binding proteins have the function of activating the OX40 pathway and inducing the activation of T cells.

FIG. 6B shows that OX40 antigen-binding proteins have CD32b crosslinking dependence.

FIG. 7 shows that the antibodies of the present application specifically bind to CHO-K1/0X40 cells, but not to other members of the TNF tumor necrosis factor receptor superfamily.

FIG. 8A shows the structural representation of molecules with an IgG-VH tetravalent symmetric structure.

FIG. 8B shows the structural representation of molecules with a Fab-HCAb tetravalent symmetric structure.

FIG. 9 shows the results of the binding of OX40×TAA bispecific antibodies to human OX40.

FIGS. 10A-10E show the results of the binding of OX40×TAA bispecific antibodies to the corresponding human tumor-associated antigens.

FIGS. 11A-11F show that OX40×TAA bispecific antibodies all have the function of activating OX40-mediated T cell pathways under tumor cell crosslinking.

FIGS. 12A-12D show the results of heterologous mixed lymphocyte reaction of OX40×TAA bispecific antibodies.

### DETAILED DESCRIPTION

The examples shown below are intended to illustrate specific embodiments of the present invention and are not intended to limit the scope of the specification or claims in any way. The examples do not include detailed descriptions of conventional methods, such as those methods for constructing vectors and plasmids, methods for inserting genes encoding proteins into such vectors and plasmids, or methods for introducing plasmids into host cells. Such methods are well known to those of ordinary skill in the art and are described in numerous publications, including Sambrook, J., Fritsch, E. F. and Maniais, T. (1989) Molecular Cloning: A Laboratory Manual, 2nd edition, Cold spring Harbor Laboratory Press.

### Example 1. Acquisition of Fully Human Anti-OX40 HCAb Antibodies

The Harbour HCAb mouse (Harbour Antibodies BV, WO 2002/085945 A3) is a transgenic mouse carrying an immune repertoire of human immunoglobulins, capable of producing novel “heavy chain”-only antibodies that are only half the size of conventional IgG antibodies. The antibodies produced have only human antibody “heavy chain” variable domains and mouse Fc constant domains. Due to the absence of light chain, this antibody almost solves the problems of light chain mismatch and heterodimerization, allowing the technical platform to develop products that are difficult to realize by the conventional antibody platform.

### Example 1.1. Immunization of HCAb Mice

The transgenic Harbour HCAb human antibody mice aged 6-8 weeks were subjected to multiple rounds of immunization through 2 immunization schemes. Specifically: as immunization scheme 1, immunization was performed with a recombinant human OX40-ECD-Fc (ChemPartner, #21127-022) antigenic protein. In each round of immunization, each mouse received a subcutaneous inguinal injection or intraperitoneal injection of 100 μL in total. In the first round of immunization, each mouse received the immunization with an immunogenic reagent prepared by mixing 50 μg of antigenic protein with complete Freund's adjuvant (Sigma, #F5881) in a 1:1 volume ratio. In each subsequent round of booster immunization, each mouse received an immunization with an immunogenic reagent prepared by mixing 25 μg of antigenic protein with Ribi adjuvant (Sigma Adjuvant System, Sigma, #S6322). As immunization scheme 2, immunization was performed with an HEK293/0X40 (ChemPartner, Shanghai) stable cell line overexpressing human OX40. In each round of immunization, each mouse received an intraperitoneal injection of 2×10⁶ cell suspension. The interval between rounds of booster immunization was at least two weeks. In general, there are no more than five rounds of booster immunizations. The immunization was performed at days 0, 14, 28, 42, 56 and 70; and the antibody titer in serum of mice was determined at days 49 and 77. The last round of booster immunization was performed at a dose of 25 μg of OX40-ECD-Fc (ChemPartner, #21127-022) antigenic protein per mouse 5 days before the isolation of HCAb mouse splenic B cells.

Blood of mice was collected, diluted in a 10-fold gradient to obtain 5 concentrations (1:100, 1:1000, 1:10000, 1:100000, 1:1000000), and determined for the titer of anti-human OX40 in the blood of mice by an ELISA assay (as described in Example 2) in an ELISA plate coated with human OX40-ECD-Fc. The blood of mice at two concentrations (1:100 and 1:1000) was determined for the specific reactivity to CHO-K1/hOX40 cells (Chempartner, Shanghai) and CHO-K1 blast cells highly expressing OX40 by flow cytometry (as described in Example 3). Serum of mice before immunization was used as a blank control group (PB).

### Example 1.2. Acquisition of Sequences of Anti-OX40 HCAb Antibodies

When the titer of the OX40-specific antibody in the serum of mice was determined to reach a certain level, spleen cells of the mice were taken from which B cells were isolated, and the populations of CD138-positive plasma cells and human OX40 antigen-positive B cells were sorted using a BD flow sorter (BD Biosciences, FACS Ariall Cell Sorter). The RNA of the B cells was extracted and reversely transcribed into cDNA (SuperScript IV First-Strand synthesis system, Invitrogen, #18091200), and human VH genes were amplified by PCR using specific primers. PCR forward primer was 5′-GGTGTCCAGTGTSAGGTGCAGCTG-3′ (SEQ ID NO: 255) and PCR reverse primer was 5′-AATCCCTGGGCACTGAAGAGACGGTGACC-3′ (SEQ ID NO: 256). The amplified VH gene fragments were constructed into a mammalian cell expression plasmid pCAG vectors encoding the sequence of the heavy chain Fc domain of the human IgG1 antibody.

Mammal host cells (e.g., human embryonic kidney cell HEK293) were transfected with the constructed plasmids and allowed to express HCAb antibodies. The binding of the HCAb-expressed supernatant to a stable cell line CHO-K1/0X40 (CHO-K1/hu OX40, (Genscript, #M00561)) overexpressing human OX40 was determined, while screening was performed by a Mirrorball® fluorescent cytometer (SPT Labtech Ltd.) with a positive antibody (Pogalizumab) used as a positive control. The specific procedures were as follows: CHO-K1/OX40 cells were washed with a serum-free F12K medium (Thermo, #21127022) and resuspended in a serum-free medium to 1×10⁶/mL. Draq5 fluorescence probes (Cell Signaling Technology, #4048L) (1 μL of Draq5 added to 1 mL of CHO-K1/OX40 cells, diluted in a 1:1000 ratio) was added, and the mixture was incubated away from light for 30 min. After centrifugation, the cells were washed with a medium and the cell density was adjusted to 1×10⁵ cells/mL. Then, Alexa Fluor® 488, AffiniPure Goat Anti-Human IgG, Fcγ Fragment Specific secondary antibody (Jackson ImmunoResearch Laboratories Inc., #109-545-098) diluted in a 1:1000 ratio was added, and the mixture was added to a 384 well plate (Greiner Bio One, #781091) at 30 μL/well. Then, the positive control or HCAB-expressed supernatant was added to the 384-well plate at 10 μL/well, and the mixture was incubated for 2 h. Fluorescence values were read on a Mirrorball instrument. The positive clonal antibodies were further determined for the cross-binding activity to a human OX40 protein (Acro biosystem, #OX0-H5224) and a cynomolgus monkey OX40 protein (Novoprotein, #CB17) by ELISA assay. Meanwhile, they were further determined for the binding activity to CHO-K1/hu OX40 cells by FACS. The nucleotide sequences of the clonal antibodies encoding the variable domains of the antibody molecules and the corresponding amino acid sequences were obtained through conventional sequencing means. Plasmids containing the remaining sequenced clonal antibodies were transfected into HEK293 cells after the removal of the repeated sequences for expression, and the obtained supernatant was again subjected to NF-kb functional assays, thus obtaining 64 functional fully human OX40 monoclonal antibodies with unique sequences that simultaneously bind to CHO-K1/hu OX40 and cynomolgus monkey OX40 proteins. According to the binding ability to human and monkey cells and the NF-κb functional assay results, top 23 antibodies in the overall rankings were selected for recombinant expression.

It is well known to those skilled in the art that the CDRs of an antibody can be defined in the art using a variety of methods, such as the Kabat scheme based on sequence variability (see Kabat et al., Sequences of Proteins of Immunological Interest, Fifth Edition, National Institutes of Health (U.S.), Bethesda, Maryland (1991)), and the Chothia scheme based on the location of the structural loop regions (see J Mol Biol 273: 927-948, 1997). In the present application, the Combined scheme comprising the Kabat scheme and the Chothia scheme can also be used to determine the amino acid residues in a variable domain sequence. The Combined scheme combines the Kabat scheme with the Chothia scheme to obtain a larger range, which is detailed in Table a of the summary of the present invention. The information on the sequences of the 23 antibodies obtained by sequencing is shown in the Table 1 below (PR002055-PR002077).

### Example 1.3. Optimization of HCAb Antibody Sequences to Improve Binding Affinity for OX40

In this example, the binding affinity of the HCAb antibody PR002067 for OX40 was improved by an antibody engineering method (a yeast display library of antibody mutations). In this example, the CDR sequences of the antibody variable domains were analyzed according to the Chothia scheme. Mutations were randomly introduced into three CDRs of PR002067 to establish yeast display libraries of mutations of 3 CDRs (CDR1, CDR2 and CDR3). The affinity maturation sorting was divided into four rounds.

In the first round, yeast cells with binding ability in 3 mutation libraries were enriched by MACS, then expanded, and induced to serve as yeast cells for the first round of FACS sorting. In the second round, yeast cells with stronger binding ability were sorted using 0.2 nM Bio-hu OX40-his (Acro biosystem, #TN4-H82E4), then collected and expanded, and induced to serve as yeast cells for next round of sorting; in the third round, yeast cells with stronger binding ability were sorted using 0.02 nM Bio-hu OX40-his at a reduced concentration, then collected and expanded, and induced to serve as yeast cells for next round of sorting; in the fourth round, yeast cells with stronger binding ability were sorted using 0.006 nM Bio-huOX40-his at a further reduced concentration. Finally, the yeast cells sorted in the fourth round were sent for sequencing to find hot spots for random combination. Variant molecules were then prepared by conventional recombinant protein expression and purification techniques, with the corresponding sequence numbers listed in Table 1 (PR005362-PR005392) and the corresponding CDR sequences listed in Tables 1-1 (PR005362-PR005392). Finally, the binding ability of the recombinant mutant molecules was determined by FACS, BLI and other methods.

TABLE 1
Sequence numbers of anti-OX40 antibodies

| Antibody | Heavy | | | | | | | | |
|---|---|---|---|---|---|---|---|---|---|
| No. | chain | VH | FWR1 | HCDR1 | FWR2 | HCDR2 | FWR3 | HCDR3 | FWR4 |
| PR002055 | 208 | 142 | 4 | 13 | 28 | 42 | 64 | 82 | 102 |
| PR002056 | 209 | 143 | 4 | 10 | 29 | 42 | 65 | 83 | 102 |
| PR002057 | 210 | 144 | 4 | 14 | 29 | 42 | 66 | 83 | 102 |
| PR002058 | 211 | 145 | 4 | 10 | 29 | 43 | 66 | 84 | 102 |
| PR002059 | 212 | 146 | 5 | 13 | 30 | 44 | 67 | 82 | 102 |
| PR002060 | 213 | 147 | 4 | 15 | 29 | 44 | 68 | 83 | 102 |
| PR002061 | 214 | 148 | 4 | 10 | 29 | 44 | 64 | 83 | 102 |
| PR002062 | 215 | 149 | 4 | 10 | 31 | 45 | 69 | 83 | 102 |
| PR002063 | 216 | 150 | 4 | 10 | 32 | 42 | 69 | 83 | 103 |
| PR002064 | 217 | 151 | 6 | 10 | 31 | 45 | 69 | 83 | 102 |
| PR002065 | 218 | 152 | 1 | 10 | 29 | 46 | 70 | 85 | 102 |
| PR002066 | 219 | 153 | 7 | 10 | 29 | 44 | 67 | 83 | 104 |
| PR002067 | 220 | 154 | 4 | 10 | 29 | 44 | 71 | 86 | 102 |
| PR002068 | 221 | 155 | 4 | 16 | 33 | 42 | 65 | 82 | 104 |
| PR002069 | 222 | 156 | 4 | 10 | 30 | 47 | 67 | 87 | 102 |
| PR002070 | 223 | 157 | 7 | 16 | 29 | 44 | 72 | 83 | 102 |
| PR002071 | 224 | 158 | 1 | 10 | 29 | 42 | 65 | 83 | 102 |
| PR002072 | 225 | 159 | 4 | 10 | 34 | 45 | 73 | 88 | 105 |
| PR002073 | 226 | 160 | 1 | 10 | 29 | 43 | 66 | 84 | 102 |
| PR002074 | 227 | 161 | 4 | 10 | 35 | 48 | 74 | 89 | 102 |
| PR002075 | 228 | 162 | 8 | 10 | 29 | 49 | 64 | 90 | 103 |
| PR002076 | 229 | 163 | 4 | 10 | 29 | 49 | 64 | 83 | 103 |
| PR002077 | 230 | 164 | 4 | 10 | 29 | 50 | 67 | 90 | 102 |
| PR005362 | 234 | 168 | 4 | 20 | 29 | 44 | 71 | 86 | 102 |
| PR005363 | 235 | 169 | 4 | 21 | 29 | 44 | 71 | 86 | 102 |
| PR005364 | 236 | 170 | 4 | 10 | 29 | 54 | 78 | 86 | 102 |
| PR005365 | 237 | 171 | 4 | 10 | 29 | 55 | 78 | 86 | 102 |
| PR005366 | 238 | 172 | 4 | 10 | 29 | 56 | 78 | 86 | 102 |
| PR005367 | 239 | 173 | 4 | 10 | 29 | 42 | 78 | 86 | 102 |
| PR005368 | 240 | 174 | 4 | 10 | 29 | 57 | 78 | 86 | 102 |
| PR005369 | 241 | 175 | 4 | 10 | 29 | 58 | 78 | 86 | 102 |
| PR005370 | 242 | 176 | 4 | 10 | 29 | 59 | 78 | 86 | 102 |
| PR005371 | 243 | 177 | 4 | 10 | 29 | 60 | 78 | 86 | 102 |
| PR005372 | 244 | 178 | 4 | 10 | 29 | 44 | 71 | 94 | 102 |
| PR005373 | 245 | 179 | 4 | 10 | 29 | 44 | 71 | 95 | 102 |
| PR005374 | 246 | 180 | 4 | 10 | 29 | 44 | 71 | 96 | 102 |
| PR005375 | 247 | 181 | 4 | 10 | 29 | 44 | 71 | 97 | 102 |
| PR005376 | 248 | 182 | 4 | 10 | 29 | 44 | 71 | 98 | 102 |
| PR005377 | 249 | 183 | 4 | 10 | 29 | 44 | 71 | 99 | 102 |
| PR005378 | 250 | 184 | 4 | 10 | 29 | 54 | 78 | 95 | 102 |
| PR005379 | 251 | 185 | 4 | 10 | 29 | 54 | 78 | 97 | 102 |
| PR005380 | 252 | 186 | 4 | 10 | 29 | 55 | 78 | 95 | 102 |
| PR005381 | 253 | 187 | 4 | 10 | 29 | 55 | 78 | 97 | 102 |
| PR005382 | 254 | 188 | 4 | 10 | 29 | 56 | 78 | 95 | 102 |
| PR005383 | 255 | 189 | 4 | 10 | 29 | 56 | 78 | 97 | 102 |
| PR005384 | 256 | 190 | 4 | 22 | 29 | 54 | 78 | 86 | 102 |
| PR005385 | 257 | 191 | 4 | 23 | 29 | 54 | 78 | 86 | 102 |
| PR005386 | 258 | 192 | 4 | 24 | 29 | 54 | 78 | 86 | 102 |
| PR005387 | 259 | 193 | 4 | 21 | 29 | 54 | 78 | 86 | 102 |
| PR005388 | 260 | 194 | 4 | 10 | 29 | 54 | 78 | 99 | 102 |
| PR005389 | 261 | 195 | 4 | 22 | 29 | 54 | 78 | 99 | 102 |
| PR005390 | 262 | 196 | 4 | 23 | 29 | 54 | 78 | 99 | 102 |
| PR005391 | 263 | 197 | 4 | 24 | 29 | 54 | 78 | 99 | 102 |
| PR005392 | 264 | 198 | 4 | 21 | 29 | 54 | 78 | 99 | 102 |

TABLE 1-1
CDR sequences of anti-OX40 antibodies

| Antibody No. | HCDR1 | HCDR2 | HCDR3 |
|---|---|---|---|
| PR002055 | GLTFSSY | SGGGGS | GMTGSTDVDY |
| PR002056 | GFTFSSY | SGGGGS | GMTGTTDVDY |
| PR002057 | GFIFSSY | SGGGGS | GMTGTTDVDY |
| PR002058 | GFTFSSY | SGGSGS | GVTGTDFDF |
| PR002059 | GLTFSSY | SGRGGS | GMTGSTDVDY |
| PR002060 | GFTFSDY | SGRGGS | GMTGTTDVDY |
| PR002061 | GFTFSSY | SGRGGS | GMTGTTDVDY |
| PR002062 | GFTFSSY | SGSGGS | GMTGTTDVDY |
| PR002063 | GFTFSSY | SGGGGS | GMTGTTDVDY |
| PR002064 | GFTFSSY | SGSGGS | GMTGTTDVDY |
| PR002065 | GFTFSSY | SGRGDI | GMTGSTDVDF |
| PR002066 | GFTFSSY | SGRGGS | GMTGTTDVDY |
| PR002067 | GFTFSSY | SGRGGS | GTTGTTDVDY |
| PR002068 | GFSFSSY | SGGGGS | GMTGSTDVDY |
| PR002069 | GFTFSSY | SGGGGN | GVTGTTDVDY |
| PR002070 | GFSFSSY | SGRGGS | GMTGTTDVDY |
| PR002071 | GFTFSSY | SGGGGS | GMTGTTDVDY |
| PR002072 | GFTFSSY | SGSGGS | GMTGTTDVDF |
| PR002073 | GFTFSSY | SGGSGS | GVTGTDFDF |
| PR002074 | GFTFSSY | SGGGNN | GWELPLLEN |
| PR002075 | GFTFSSY | SGSGGN | GITGTTDVDY |
| PR002076 | GFTFSSY | SGSGGN | GMTGTTDVDY |
| PR002077 | GFTFSSY | SGRGNI | GITGTTDVDY |
| PR005362 | GLPFDCY | SGRGGS | GTTGTTDVDY |
| PR005363 | GLPFDSY | SGRGGS | GTTGTTDVDY |
| PR005364 | GFTFSSY | SGRGGQ | GTTGTTDVDY |
| PR005365 | GFTFSSY | SGLGGQ | GTTGTTDVDY |
| PR005366 | GFTFSSY | SGRSGQ | GTTGTTDVDY |
| PR005367 | GFTFSSY | SGGGGS | GTTGTTDVDY |
| PR005368 | GFTFSSY | SGLGGS | GTTGTTDVDY |
| PR005369 | GFTFSSY | TGRGGQ | GTTGTTDVDY |
| PR005370 | GFTFSSY | SGHGGT | GTTGTTDVDY |
| PR005371 | GFTFSSY | SGRGGT | GTTGTTDVDY |
| PR005372 | GFTFSSY | SGRGGS | GTTGSWDVCY |
| PR005373 | GFTFSSY | SGRGGS | GTTGTWDVDW |
| PR005374 | GFTFSSY | SGRGGS | GTTGSWDVDW |
| PR005375 | GFTFSSY | SGRGGS | GTTGSYDVDW |
| PR005376 | GFTFSSY | SGRGGS | GTTGSTDVDW |
| PR005377 | GFTFSSY | SGRGGS | GTTGTWDVDY |
| PR005378 | GFTFSSY | SGRGGQ | GTTGTWDVDW |
| PR005379 | GFTFSSY | SGRGGQ | GTTGSYDVDW |
| PR005380 | GFTFSSY | SGLGGQ | GTTGTWDVDW |
| PR005381 | GFTFSSY | SGLGGQ | GTTGSYDVDW |
| PR005382 | GFTFSSY | SGRSGQ | GTTGTWDVDW |
| PR005383 | GFTFSSY | SGRSGQ | GTTGSYDVDW |
| PR005384 | GLPFSSY | SGRGGQ | GTTGTTDVDY |
| PR005385 | GLTFDSY | SGRGGQ | GTTGTTDVDY |
| PR005386 | GFPFDSY | SGRGGQ | GTTGTTDVDY |
| PR005387 | GLPFDSY | SGRGGQ | GTTGTTDVDY |
| PR005388 | GFTFSSY | SGRGGQ | GTTGTWDVDY |
| PR005389 | GLPFSSY | SGRGGQ | GTTGTWDVDY |
| PR005390 | GLTFDSY | SGRGGQ | GTTGTWDVDY |
| PR005391 | GFPFDSY | SGRGGQ | GTTGTWDVDY |
| PR005392 | GLPFDSY | SGRGGQ | GTTGTWDVDY |

### Example 1.4. Preparation of Fully Human Recombinant Anti-OX40 Antibodies

Mammalian host cells (e.g., human embryonic kidney cell HEK293) were transfected with plasmids encoding HCAb antibodies, and purified anti-OX40 recombinant heavy-chain antibodies could be obtained using conventional recombinant protein expression and purification techniques. Specifically, HEK293 cells were expanded in FreeStyle™ F17 Expression Medium (Thermo, #A1383504). Before transient transfection, the cells were adjusted to a concentration of 6×10⁵ cells/mL, and cultured in a shaker at 37° C. with 8% CO₂ for 24 h to make a concentration of 1.2×10⁶ cells/mL. 30 mL of the cultured cells were taken, and 30 μg of the above plasmids encoding HCAb heavy chains were dissolved in 1.5 mL of Opti-MEM serum-free medium (Thermo, #31985088). Then 120 μL of 1 mg/mL PEI (Polysciences, Inc, #23966-2) was dissolved in 1.5 mL of Opti-MEM, and the mixture was left to stand for 5 min. PEI was slowly added to the plasmids, and the mixture was incubated at room temperature for 10 min. The mixed solution of plasmids and PEI was slowly added dropwise while shaking the culture flask, and the cells were cultured in a shaker at 37° C. with 8% CO₂ for 5 days. Cell viability was measured after 5 days. The culture was collected and centrifuged at 3300 g for 10 min, and then the supernatant was collected and centrifuged at high speed to remove impurities. A gravity column (Bio-Rad, #7311550) containing MabSelect™ (GE Healthcare Life Science, #71-5020-91 AE) was equilibrated with PBS (pH 7.4) and rinsed with 2-5 column volumes of PBS. The supernatant sample was loaded onto a column. The column was rinsed with 5-10 column volumes of PBS. The target protein was eluted with 0.1 M glycine (pH 3.5). The eluate was adjusted to neutrality with Tris-HCl (pH 8.0), and concentrated and buffer exchanged into PBS buffer with an ultrafiltration tube (Millipore, #UFC901024) to obtain a purified anti-human OX40 HCAb monoclonal antibody solution. The antibody concentration was determined by measuring the absorbance at 280 nm using NanoDrop, and the antibody purity was determined by SEC-HPLC and SDS-PAGE.

Meanwhile, a positive control anti-OX40 antibody Pogalizumab analog was produced in the present application, with the corresponding antibody number of PR003475. Its corresponding amino acid sequences were found in the IMGT database, in which the amino acid sequence of heavy chain was set forth in SEQ ID NO: 233, and the amino acid sequence of the light chain was set forth in SEQ ID NO: 270.

### Example 1.5. Analysis of Protein Purity and Polymers by HPLC-SEC

Analytical size-exclusion chromatography (SEC) was used to analyze the obtained protein samples for purity and polymer forms. An analytical chromatography column TSKgel G3000SWx1 (Tosoh Bioscience, 08541, 5 μm, 7.8 mm×30 cm) was connected to a high-performance liquid chromatograph (HPLC, model: Agilent Technologies, Agilent 1260 Infinity II) and equilibrated with a PBS buffer at room temperature for at least 1 h. A proper amount of the protein sample (at least 10 μg, with the concentration adjusted to 1 mg/mL) was filtered through a 0.22 μm membrane filter and then injected into the system, and the program for HPLC was set: the sample was eluted in the chromatography column with a PBS buffer (pH 7.4) at a flow rate of 1.0 mL/min for a maximum of 20 min. The detection wavelength was 280 nm. After being recorded, the chromatogram was integrated using ChemStation software and relevant data were calculated. An analysis was generated, with the retention time of the components with different molecular sizes in the sample reported.

### Example 1.6. Analysis of Protein Purity and Hydrophobicity by HPLC-HIC

Analytical hydrophobic interaction chromatography (HIC) was used to analyze the obtained protein samples for purity and hydrophobicity. An analytical chromatography column TSKge1 Butyl-NPR (Tosoh Bioscience, 14947, 4.6 mm×3.5 cm) was connected to a high-performance liquid chromatograph (HPLC, model: Agilent Technologies, Agilent 1260 Infinity II) and equilibrated with a PBS buffer at room temperature for at least 1 h. The program for HPLC was set: a linear gradient from 100% mobile phase A (20 mM histidine, 1.8 M ammonium sulfate, pH 6.0) to 100% mobile phase B (20 mM histidine, pH 6.0) over 16 min; flow rate: 0.7 mL/min; protein sample concentration: 1 mg/mL; and injection volume: 20 μL. The detection wavelength was 280 nm. After being recorded, the chromatogram was integrated using ChemStation software and relevant data were calculated. An analysis was generated, with the retention time of the components with different molecular sizes in the sample reported.

### Example 1.7. Determination of Thermostability of Protein Molecules by DSF

Differential scanning fluorimetry (DSF) is a commonly used high-throughput method for determining the thermostability of proteins. In this method, changes in the fluorescence intensity of the dye that binds to unfolded protein molecules were monitored using a real-time quantitative fluorescence PCR instrument to reflect the denaturation process of the protein and thus to reflect the thermostability of the protein. In this example, the thermal denaturation temperature (Tm) of a protein molecule was measured by DSF. 10 μg of protein was added to a 96-well PCR plate (Thermo, AB-07001W), followed by the addition of 2 μL of 100×diluted dye SYPRO™ (Invitrogen, #2008138), and then a buffer was added to make a final volume of 40 μL per well. The PCR plate was sealed, placed in a real-time quantitative fluorescence PCR instrument (Bio-Rad, model: CFX96 PCR System), and incubated at 25° C. for 5 min, then at a temperature gradually increased from 25° C. to 95° C. at a gradient of 0.2° C./0.2 min, and at a temperature decreased to 25° C. at the end of the test. The FRET scanning mode was used and data analysis was performed using Bio-Rad CFX Maestro software to calculate the Tm of the sample. The results are shown in Table 2 below.

TABLE 2
Physicochemical properties of OX40 antibodies

| | | Yield | | | | | | |
|---|---|---|---|---|---|---|---|---|
| | Expression | (mg/L) | | | Endotoxin | | HPLC-HIC | |
| Recombinant | system and | after first | HPLC-SEC | Concentration | level | | retention | DSF |
| antibody | volume | purification | purity (%) | (mg/mL) | (EU/mg) | Total(mg) | time (min) | TM1(° C.) |
| PR002055 | HEK293 | 56.6 | 99.64 | 3.77 | <1 | 2.26 | 17.931 | 56.2 |
| | (40 ml) | | | | | | | |
| PR002056 | HEK293 | 147 | 98.53 | 1.96 | <1 | 5.88 | 17.872 | 51.2 |
| | (40 ml) | | | | | | | |
| PR002057 | HEK293 | 62 | 100 | 3.1 | <1 | 2.48 | 17.686 | 47.2 |
| | (40 ml) | | | | | | | |
| PR002058 | HEK293 | 34.4 | 99.03 | 2.29 | <1 | 1.37 | 16.899 | 47 |
| | (40 ml) | | | | | | | |
| PR002059 | HEK293 | 147.8 | 100 | 1.97 | <1 | 5.91 | 16.646 | 57.4 |
| | (40 ml) | | | | | | | |
| PR002060 | HEK293 | 32.7 | 99 | 2.18 | <1 | 1.31 | 17.731 | 57 |
| | (40 ml) | | | | | | | |
| PR002061 | HEK293 | 128 | 99.6 | 6.4 | <1 | 5.12 | 17.67 | 53.2 |
| | (40 ml) | | | | | | | |
| PR002062 | HEK293 | 126 | 99.52 | 6.3 | <1 | 5.04 | 17.148 | 51.8 |
| | (40 ml) | | | | | | | |
| PR002063 | HEK293 | 137.5 | 99.3 | 5.5 | <1 | 5.5 | 18.109 | 51.8 |
| | (40 ml) | | | | | | | |
| PR002064 | HEK293 | 132.5 | 99.47 | 5.3 | <1 | 5.3 | 16.989 | 51.4 |
| | (40 ml) | | | | | | | |
| PR002065 | HEK293 | 90 | 100 | 3.6 | <1 | 3.6 | 19.235 | 57.8 |
| | (40 ml) | | | | | | | |
| PR002066 | HEK293 | 142.5 | 99.19 | 5.7 | <1 | 5.7 | 17.93 | 53 |
| | (40 ml) | | | | | | | |
| PR002067 | HEK293 | 87.5 | 99.29 | 3.5 | <1 | 3.5 | 17.469 | 45.4 |
| | (40 ml) | | | | | | | |
| PR002068 | HEK293 | 8.4 | n/a | 0.56 | <1 | 0.34 | — | 45 |
| | (40 ml) | | | | | | | |
| PR002069 | HEK293 | 38.4 | 99.54 | 2.56 | <1 | 1.54 | 16.929 | 53.4 |
| | (40 ml) | | | | | | | |
| PR002070 | HEK293 | 120 | 100 | 4.8 | <1 | 4.8 | 17.531 | 51.8 |
| | (40 ml) | | | | | | | |
| PR002071 | HEK293 | 35.4 | 99.03 | 2.36 | <1 | 1.42 | 17.867 | 52.6 |
| | (40 ml) | | | | | | | |
| PR002072 | HEK293 | 49.5 | 99.18 | 3.3 | <1 | 1.98 | 18.387 | 54.2 |
| | (40 ml) | | | | | | | |
| PR002073 | HEK293 | 37.8 | 100 | 2.52 | <1 | 1.51 | 16.862 | 48 |
| | (40 ml) | | | | | | | |
| PR002074 | HEK293 | 37.2 | 96.7 | 2.48 | <1 | 1.49 | 18.971 | 56.2 |
| | (40 ml) | | | | | | | |
| PR002075 | HEK293 | 84 | 98 | 4.2 | <1 | 3.36 | 18.137 | 53.2 |
| | (40 ml) | | | | | | | |
| PR002076 | HEK293 | 117.5 | 100 | 4.7 | <1 | 4.7 | 17.688 | 56.8 |
| | (40 ml) | | | | | | | |
| PR002077 | HEK293 | 95 | 97.28 | 3.8 | <1 | 3.8 | 18.315 | 53.2 |
| | (40 ml) | | | | | | | |

### Example 2. Binding Ability of Anti-OX40 HCAb Monoclonal Antibodies at the Protein Level as Assayed by ELISA

This example is intended to investigate the in vitro binding activity of the anti-OX40 HCAb monoclonal antibodies prepared in Example 1 to human and cynomolgus monkey OX40 proteins. The antibody binding assay at the protein level was performed using a human OX40 protein (Acro biosystem, #OX0-H5224) and a cynomolgus monkey OX40 protein (Novoprotein, #CB17). Briefly, a 384-well plate (PerkinElmer, #6007509) was coated with 1 μg/mL human OX40 protein and a cynomolgus monkey OX40 protein dissolved in PBS at 20 μL/well, and incubated at 4° C. overnight. The next day, the 384-well plate was washed three times with PBS containing 0.05% Tween (MEDICAGO, #09-9410-100), and blocked with PBS containing 2% milk (Bio-Rad, #170-6404) at 37° C. for 1 h. The test OX40 antibodies and positive antibody (Pogalizumab) were diluted in a 4-fold gradient from the initial concentration of 10 nM. The blocked 384-well plate was washed three times with PBST, added with 10 μL of PBS or 10 μL of antibody and positive control (Pogalizumab) diluted in a 4-fold gradient, and incubated at room temperature for 1 h. The plate was washed three times, added with goat anti-human Fc horseradish peroxidase (Jackson ImmunoResearch Laboratories Inc., #109-035-098) at 20 μL/well, and incubated at 37° C. for 40 min. The plate was washed three times, added with TMB (Sera Care, #5120-0077) at 20 μL/well, and incubated at room temperature for 5-15 min. The plate was added with a stop buffer (BBI life sciences, #E661006-0200) at 20 μL/well, and read for OD₄₅₀₋₆₅₀ values using a plate reader (Molecular Devices, model: SpectraMax Plus). The values were analyzed with Graphad 8.0 and plotted.

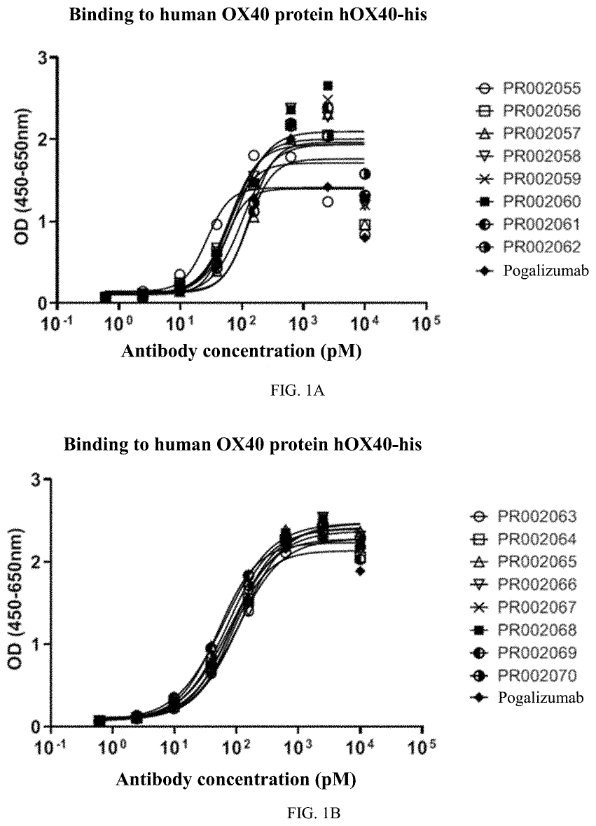

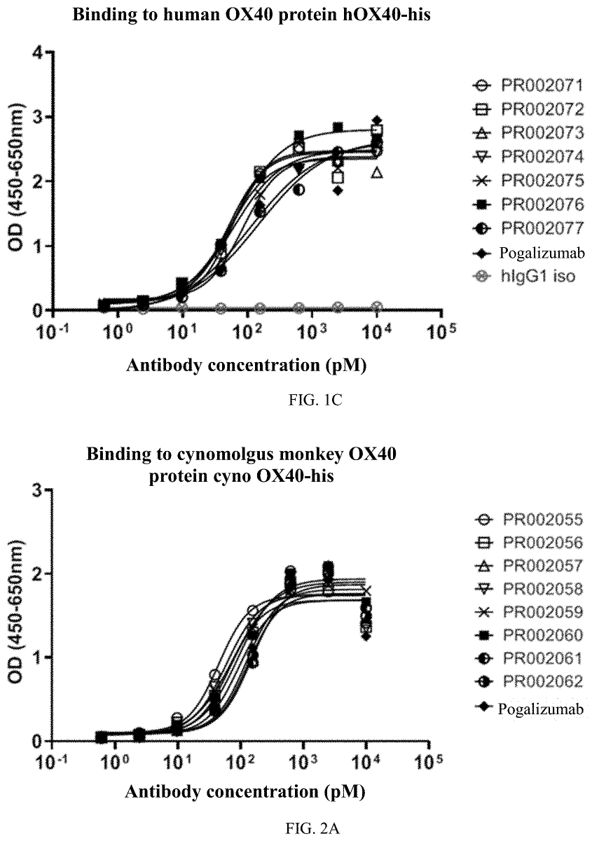

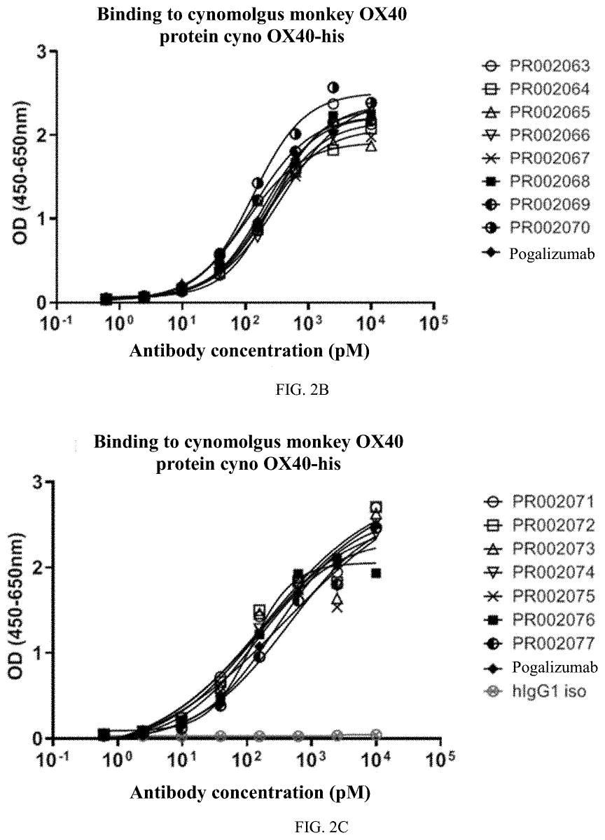

As shown in FIGS. 1 and 2 and Tables 3 and 4, the OX40 antibodies of this example were all able to bind to human OX40 and cynomolgus monkey (cyno) OX40 proteins, with the detected antibody binding ability increasing in a positive correlation with the antibody concentration. In the comparison with the reference antibody Tab ((Pogalizumab), conventional IgG antibody, with heavy chain and light chain sequences set forth in SEQ ID NO: 233 and SEQ ID NO: 270, respectively), PR002055, PR002056, PR002058, PR002059, PR002065, PR002069, PR002070, PR002074 and PR002076 had an EC50 value for binding to human OX40 and cynomolgus monkey OX40 proteins comparable to or lower than Tab (Pogalizumab), indicating that those antibodies can bind to human OX40 more sensitively at lower concentrations, among which PR002055 is the best, with an EC50 of less than 50 pM.

TABLE 3
Binding activity of OX40 antibodies to a human OX40 protein

| Antibody | EC50 (pM) | Maximum OD |
|---|---|---|
| PR002055 | 26.84 | 1.412 |
| PR002056 | 56.97 | 1.713 |
| PR002057 | 124.1 | 1.76 |
| PR002058 | 62.92 | 1.935 |
| PR002059 | 69.52 | 2.003 |
| PR002060 | 82.81 | 2.094 |
| PR002061 | 134.4 | 1.963 |
| PR002062 | 103.1 | 1.939 |
| PR002063 | 100.9 | 2.281 |
| PR002064 | 81.43 | 2.28 |
| PR002065 | 63.88 | 2.474 |
| PR002066 | 118.1 | 2.47 |
| PR002067 | 80.6 | 2.378 |
| PR002068 | 91.97 | 2.418 |
| PR002069 | 50.97 | 2.236 |
| PR002070 | 72.89 | 2.405 |
| PR002071 | 50.16 | 2.452 |
| PR002072 | 57.72 | 2.471 |
| PR002073 | 85.02 | 2.38 |
| PR002074 | 51.15 | 2.349 |
| PR002075 | 61.84 | 2.484 |
| PR002076 | 64.97 | 2.8 |
| PR002077 | 145.6 | 2.665 |
| Pogalizumab | 82.59 | 2.134 |

TABLE 4
Binding activity of OX40 antibodies
to a cynomolgus monkey OX40 protein

| Antibody | EC50 (pM) | Maximum OD |
|---|---|---|
| PR002055 | 45.22 | 1.743 |
| PR002056 | 68.19 | 1.685 |
| PR002057 | 132.8 | 1.764 |
| PR002058 | 59.74 | 1.761 |
| PR002059 | 77.35 | 1.904 |
| PR002060 | 93.8 | 1.94 |
| PR002061 | 151.8 | 1.872 |
| PR002062 | 125.6 | 1.815 |
| PR002063 | 275.8 | 2.318 |
| PR002064 | 217.9 | 2.079 |
| PR002065 | 103.5 | 1.918 |
| PR002066 | 369.7 | 2.444 |
| PR002067 | 232 | 2.163 |
| PR002068 | 253.1 | 2.381 |
| PR002069 | 129 | 2.238 |
| PR002070 | 131.9 | 2.513 |
| PR002071 | 201.5 | 2.927 |
| PR002072 | 216.7 | 2.968 |
| PR002073 | 145.7 | 2.552 |
| PR002074 | 140.4 | 2.341 |
| PR002075 | 414.9 | 3.072 |
| PR002076 | 115.6 | 2.053 |
| PR002077 | 440.9 | 2.729 |
| Pogalizumab | 194.3 | 2.226 |

### Example 3. Binding Ability of Anti-OX40 HCAb Monoclonal Antibodies at the Cellular Level as Assayed by FACS

This example is intended to investigate the in vitro binding activity of anti-human OX40 HCAb monoclonal antibodies to human OX40. The antibody binding assay at the cellular level was performed using a stable CHO-K1 cell strain overexpressing human OX40 (CHO-K1/hu OX40). Briefly, the CHO-K1/hu OX40 cells were digested, resuspended in an F12K complete medium and washed once with PBS. The cell density was adjusted to 1×10⁶ cells/mL with PBS. The cells were seeded in a 96-well V-bottom plate (Corning, #3894) at 100 μL/well, and centrifuged, and then the supernatant was discarded. Then, the test antibodies diluted in a 3-fold gradient at concentrations that were 2 times the final concentrations were added at 100 μL/well. The cells were incubated at 4° C. for 1 h away from light. Thereafter, the cells in each well were rinsed twice with 100 μL of pre-cooled PBS, and centrifuged at 500 g at 4° C. for 5 min, and then the supernatant was discarded. Then, 100 μL of a fluorescent secondary antibody (Alexa Fluor 488-conjugated AffiniPure Goat Anti-Human IgG, Fcγ Fragment Specific, Jackson, #109-545-06, diluted in a 1:1000 ratio) was added to each well. The plate was incubated away from light at 4° C. for 30 min. The cells in each well were rinsed twice with 100 μL of pre-cooled PBS, and centrifuged at 500 g at 4° C. for 5 min, and then the supernatant was discarded. Finally, the cells in each well were resuspended in 200 μL of pre-cooled PBS, and the fluorescence signal values were read using a BD FACS CANTOII.

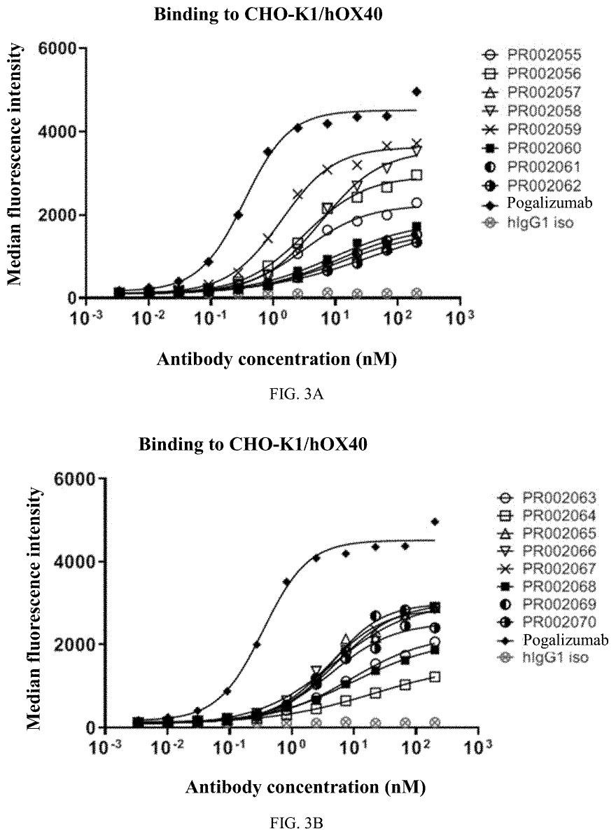

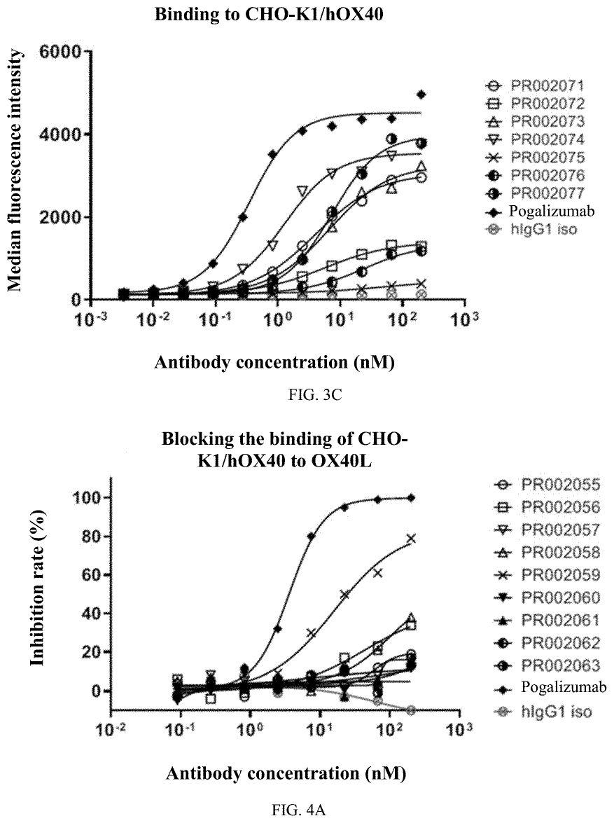

As shown in FIGS. 3A-3C and Table 5, the OX40 antibodies of the present invention were all able to bind to CHO-K1/hu OX40 cells, with the detected binding ability increasing in a positive correlation with the antibody concentration.

TABLE 5
Binding activity of OX40 antibodies to CHO-K1/hu OX40

| Antibody | EC50 (nM) | Maximum MFI |
|---|---|---|
| PR002055 | 3.18 | 2244 |
| PR002056 | 3.12 | 2928 |
| PR002057 | 12.81 | 1668 |
| PR002058 | 5.79 | 3559 |
| PR002059 | 1.35 | 3628 |
| PR002060 | 8.95 | 1857 |
| PR002061 | 10.95 | 1738 |
| PR002062 | 37.89 | 1902 |
| PR002063 | 9 | 2244 |
| PR002064 | 22.44 | 1573 |
| PR002065 | 3.82 | 2865 |
| PR002066 | 4.67 | 3012 |
| PR002067 | 5.25 | 3012 |
| PR002068 | 10.08 | 2129 |
| PR002069 | 4.43 | 2992 |
| PR002070 | 4.39 | 2532 |
| PR002071 | 4 | 3059 |
| PR002072 | 5.64 | 1405 |
| PR002073 | 6.55 | 3258 |
| PR002074 | 1.27 | 3541 |
| PR002075 | n.d. | 501 |
| PR002076 | 24.59 | 1380 |
| PR002077 | 7.22 | 3982 |
| Pogalizumab | 0.34 | 4516 |

### Example 4. Blocking of Antigen-Binding Proteins on Binding of OX40 to an OX40 Ligand

In order to investigate the in vitro blocking ability of human OX40-binding proteins to the binding of human OX40 to a human OX40 ligand (OX40L), the blocking assay on human OX40/OX40L binding was performed using a CHO-K1 cell strain overexpressing human OX40 (CHO-K1/hu OX40). Briefly, the CHO-K1/hu OX40 cells were digested and resuspended in an F-12K complete medium, and the cell density was adjusted to 1×10⁶ cells/mL. The cells were seeded in a 96-well V-bottom plate (Corning, #3894) at 100 μL/well, and centrifuged, and then the supernatant was discarded. The test antigen-binding proteins diluted in a 3-fold gradient at concentrations that were 2 times the final concentrations were added at 100 μL/well, and the mixture was well mixed, wherein the antigen-binding protein had the highest final concentration of 100 nM, and a total of 8 concentrations were obtained. Pogalizumab was used as a positive control, while hIgG1 was used as a negative control. Meanwhile, two other controls were set, i.e., a no-blocking control with no antibody and only biotin-labeled human OX40L protein and secondary antibody, and a 100% blocking control with only secondary antibody. The cells were incubated at 4° C. for 1 h away from light. Thereafter, the cells were centrifuged at 4° C. for 5 min, and then the supernatant was discarded. Then, 50 μL, of 0.1 μg/mL biotin-labeled human OX40L protein (Acro biosystem, OXL-H82Q6) was added to each well except for the 100% blocking well, and the cells were incubated away from light at 4° C. for 30 min. The cells in each well were rinsed twice with 100 μL of pre-cooled PBS, and centrifuged at 500 g at 4° C. for 5 min, and then the supernatant was discarded. A 1:200 fluorescent secondary antibody PE Streptavidin (BD Biosciences, #554061) was added at 100 μL/well, and the cells were incubated away from light at 4° C. for 30 min. The cells in each well were rinsed twice with 200 μL of pre-cooled PBS, and centrifuged at 500 g at 4° C. for 5 min, and then the supernatant was discarded. Finally, the cells in each well were resuspended in 200 μL of precooled PBS. Fluorescence signal values were read using BD FACS CANTOII, and IC50 was calculated. Inhibition rate %={average MFI of non-blocking control well−MFI value of OX40 antibody}/{(average MFI of non-blocking control well)−average MFI of 100% blocking control well)}×100%.

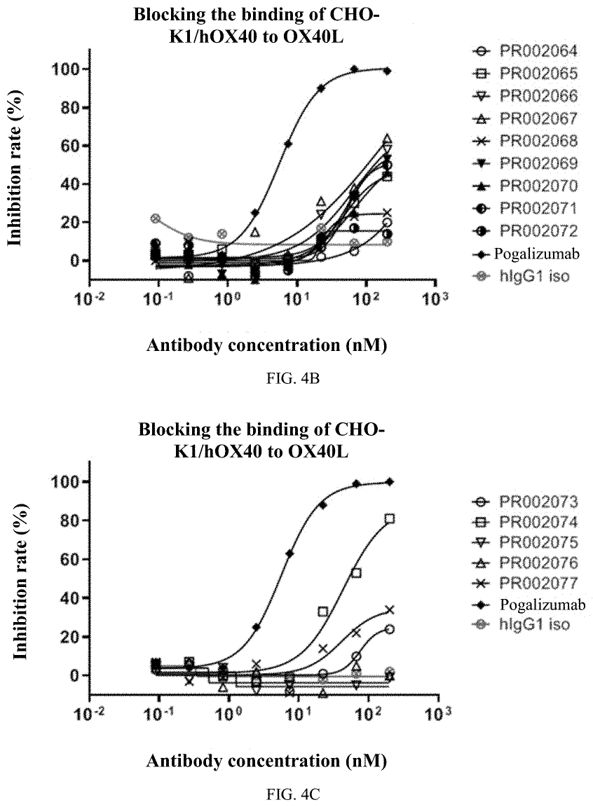

The results are shown in FIGS. 4A-4C and Table 6. FIGS. 4A-4C and Table 6 showed that the OX40 antibodies described herein (OX40 antigen-binding proteins) had relatively weak blocking ability to the binding of a human OX40 ligand to human OX40 on the cell surface. PR002059 showed comparable blocking effect to the reference antibody Tab (Pogalizumab); and the remaining antibodies showed a relatively weak blocking effect. This indicates that most of the OX40 antigen-binding proteins described herein are weak blocking or non-blocking antibodies.

TABLE 6
Blocking of antigen-binding proteins
on binding of an OX40 ligand to OX40

| Antibody | IC50 (nM) | Maximum inhibition rate (%) |
|---|---|---|
| PR002055 | 59.94 | 19 |
| PR002056 | 43.71 | 34 |
| PR002057 | n.d. | 12 |
| PR002058 | 98.1 | 38 |
| PR002059 | 15.94 | 79 |
| PR002060 | n.d. | 12 |
| PR002061 | n.d. | 16 |
| PR002062 | n.d. | 13 |
| PR002063 | n.d. | 17 |
| PR002064 | n.d. | 20 |
| PR002065 | 49.58 | 44 |
| PR002066 | 67.61 | 58 |
| PR002067 | 166.2 | 64 |
| PR002068 | 23.06 | 25 |
| PR002069 | 50.86 | 53 |
| PR002070 | 67.57 | 46 |
| PR002071 | 51.73 | 50 |
| PR002072 | n.d. | 14 |
| PR002073 | 75.21 | 24 |
| PR002074 | 43.13 | 81 |
| PR002075 | n.d. | 0 |
| PR002076 | n.d. | 0 |
| PR002077 | 41.1 | 34 |
| Pogalizumab | 5.575 | 99 |

### Example 5. Stimulation of the OX40 Signaling Pathway by OX40 Antibodies (Antigen-Binding Proteins) as Assayed Using a Reporter Gene Cell Line

CHO-K1 cells expressing CD32b (CHO-K1/CD32b) (Genscript, #M00587) or CHO-K1 (ATCC, #CCL-61) were plated in a 96-well plate (Perkin Elmer, #6005225) at 1.5×10⁴ cells/100 μL/well, and incubated in an incubator at 37° C. with 5% CO2 overnight. The next day, the supernatant was removed, the test antigen-binding proteins diluted in a 5-fold gradient at concentrations that were 2 times the final concentrations (initial final concentration: 200 nM) were added to the 96-well plate at 40 μL/well. hlgG1 was used as a negative control. HEK293 reporter cells capable of constantly expressing the luciferase reporter genes of OX40 and NF-kb response elements (HEK293/OX40/NF-kb reporter cells, BPS Biosciences, #60482) were added at 4.5×10⁴ cells/40 μL/well. The cells were incubated in an incubator at 37° C. with 5% CO2 for 6 h. Then, ONE-Glo™ luciferase reagent (Promega, #E6110) was added. The cells were incubated at room temperature for 5 min, and the luminescence values were determined using a microplate reader.

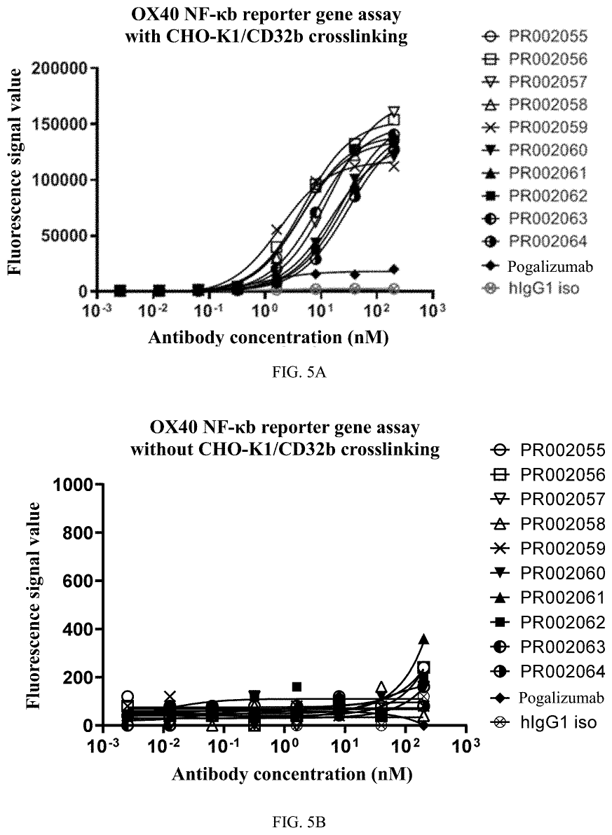

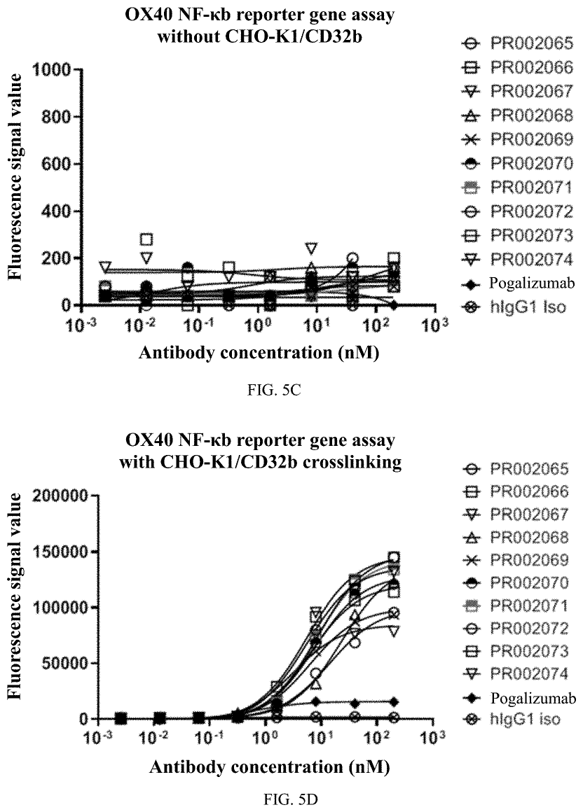

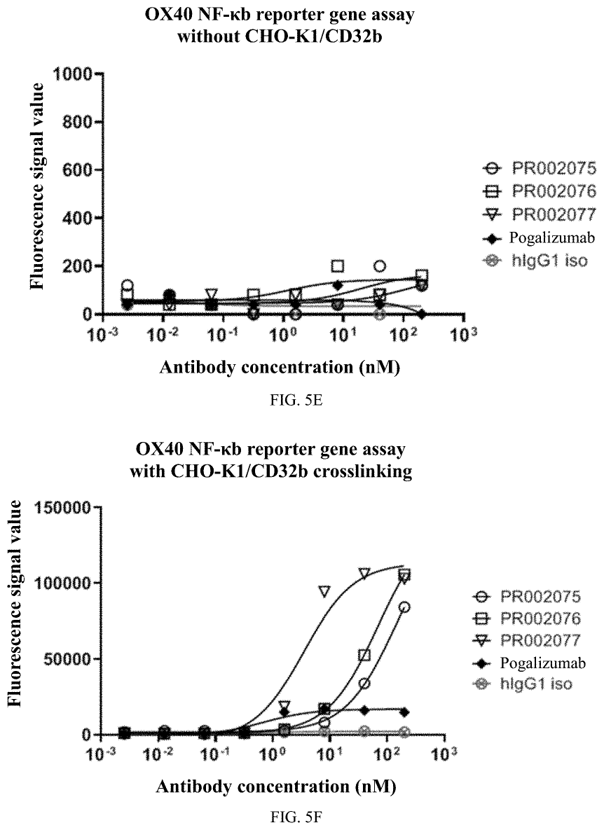

The results are shown in FIGS. 5A-5F and Table 7 below. The results in FIGS. 5A-5F and Table 7 showed that the OX40 antigen-binding proteins described herein were all CD32b crosslinking dependent. Under CHO-K1/CD32b crosslinking, the promotion of the OX40-mediated NF-κb signaling pathway by most of the OX40 antigen-binding proteins described herein increased in a positive correlation with their concentrations. In the comparison with the reference antibody Tab (Pogalizumab), all OX40 antibodies had a higher maximum luminescence value than the reference antibody, indicating that those antibodies were all able to promote greater activation of NF-κb. Among them, PR002059 was the best, and it had comparable EC₅₀ to the reference antibody, but had a higher maximum luminescence value than the reference antibody.

TABLE 7
Stimulation of the OX40 signaling
pathway by antigen-binding proteins

| | | Maximum fluorescence |
|---|---|---|
| Antibody | EC50 (nM) | signal value |
| PR002055 | 4.66 | 140624 |
| PR002056 | 4.89 | 153884 |
| PR002057 | 14.35 | 172165 |
| PR002058 | 4.29 | 135546 |
| PR002059 | 1.83 | 116834 |
| PR002060 | 15.1 | 132365 |
| PR002061 | 23.2 | 155668 |
| PR002062 | 25.22 | 148937 |
| PR002063 | 8.64 | 149686 |
| PR002064 | 30.34 | 147258 |
| PR002065 | 8.55 | 149211 |
| PR002066 | 5.45 | 146566 |
| PR002067 | 5.37 | 136921 |
| PR002068 | 22.95 | 138931 |
| PR002069 | 6.27 | 99697 |
| PR002070 | 7.6 | 129161 |
| PR002071 | 7.85 | 143752 |
| PR002072 | 14.21 | 99625 |
| PR002073 | 6.39 | 120867 |
| PR002074 | 3.08 | 84796 |
| PR002075 | 135.9 | 140219 |
| PR002076 | 67.03 | 140541 |
| PR002077 | 3.51 | 113373 |
| Pogalizumab | 0.91 | 18147 |
| hIgG1 iso | / | 2530 |

### Example 6. Antigen-Binding Proteins Capable of Activating the OX40 Pathway In Vitro

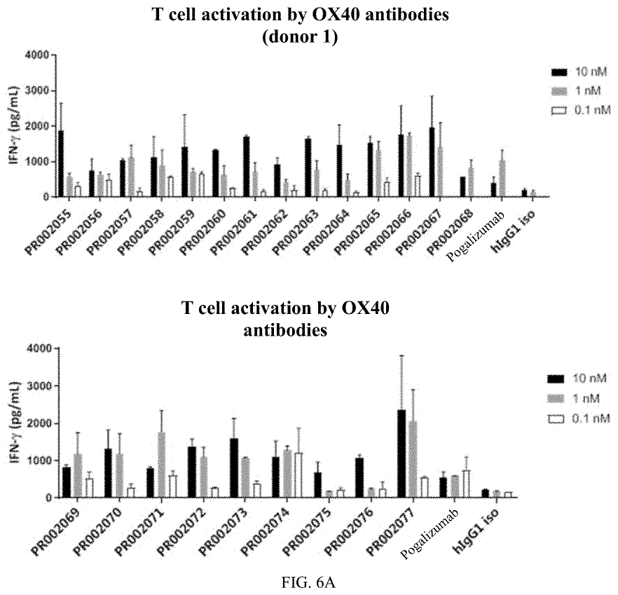

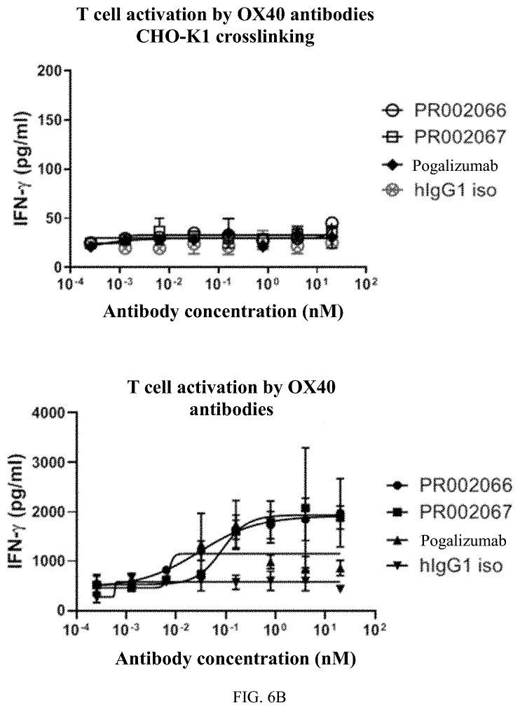

CHO-K1 (ATCC, #CCL-61) or CHO-K1-CD32b cells were treated with 10 μg/mL mitomycin (Beijing Ruitaibio, #10107409001) at 37° C. for 30 min. Then, the cells were washed 4 times with an F-12K culture medium containing 10% FBS. The two treated cells were plated in a 96-well flat-bottom plate (Corning, #3559) at 1.5×10⁴ cells/well and incubated in an incubator at 37° C. overnight. The next day, human CD3 positive T cells were isolated from human PBMCs using a MACS kit (Miltenyi Biotec, #130-096-535). The number of the cells was determined firstly, and then corresponding amounts of MACS buffer and Pan-T cell biotin antibody were added according to the number of the cells. The mixture was well mixed and left to stand at 4° C. for 5 min. Then, a corresponding amount of magnetic microbeads was added, and the mixture was left to stand at 4° C. for 10 min. What passed through the LS column were CD3 positive T cells. The previous day's culture medium was washed off the 96-well plate, and purified T cells were added at 1×10⁵ cells/well. Then, an OX40 antibody or a control antibody at a corresponding concentration was added, and OKT3 (eBiosciences, #16-0037-85) was added to make a final concentration of 0.3 μg/mL. The cells were incubated in an incubator at 37° C. with 5% CO2 for 72 h. 72 h later, the supernatant was collected and assayed for IFN-γ content using an ELISA kit (Invitrogen, #88-7316-88). The ELISA assay was performed by referring to the instructions of relevant kit. The absorbance values at 450 nm and 570 nm were read using a microplate reader (Molecular Devices, model: SpectraMax Plus), and the concentration of IFN-γ in the supernatant was calculated from the standard readings (OD₄₅₀-OD₅₇₀). The data were processed and analyzed by plotting using GraphPad Prism 8 software. The results are shown in FIG. 6A. It could be seen that most of the OX40 antigen-binding proteins described herein had the function of activating the OX40 pathway and inducing the activation of T cells, with the activation effect stronger than the reference antibody Tab (Pogalizumab). As shown in FIG. 6B and Table 8, the OX40 antigen-binding proteins of the present invention all had CD32b crosslinking dependence and had greater activation effects on T cells than the reference antibody.

TABLE 8
Activation effect of OX40 antibodies on T cells

| Antibody | EC50(nM) | Maximum IFN-γ release value |
|---|---|---|
| PR002066 | 0.03102 | 1873 |
| PR002067 | 0.08634 | 2003 |
| Pogalizumab | 0.003736 | 1142 |

### Example 7. Determination of Binding Affinity and Dissociation Constant of OX40 Antibodies to a Recombinant OX40 Protein by the BLI Method

The binding kinetics between the antigen and the antibody was analyzed by the Biolayer Interferometry (BLI) technique using an Octet Red 96e molecular interaction analyzer (Fortebio). Affinity was determined using an Octet RED96 instrument (Pall Fortebio) and a ProA avidin sensor (Pall ForteBio, #18-5010) according to the detailed procedures and methods provided by the manufacturer.

The ProA avidin sensors placed in a column were equilibrated in an assay buffer for 10 min, and then were used to capture the OX40 antibodies at 200 nM at a capture height of 0.8 nM; the ProA sensors were equilibrated in a buffer for 120 s, and then were associated with human OX40 proteins or cynomolgus monkey OX40 proteins diluted in a 2-fold gradient (OX40 HCAB at 200-6.25 nM and 0 nM; Pogalizumab at 25-1.56 nM and 0 nM) for 180 s, and dissociated for 800 s (PR002063, PR002065, PR002066 and PR002077 dissociated for 400 s from cynomolgus monkey proteins); finally, the ProA sensors were immersed into 10 mM glycine-hydrochloric acid (pH 1.5) solution for regeneration to elute the proteins associated with the sensors. The association and dissociation signals between the OX40 antibodies and OX40 proteins were recorded by Octet Red 96 in real time. When data analysis was performed using Octet Data Analysis software (Fortebio, version 11.0), 0 nM was used as a reference hole, and reference subtraction was performed; the “1:1 Global fitting” method was selected to fit the data, and the kinetics parameters of the binding of antigens to antigen-binding proteins were calculated, with kon(1/Ms) values, kdis(1/s) values and KD(M) values obtained. The results are as shown in Table 9. It could be seen that most of the OX40 antigen-binding proteins described herein had slightly higher KD(M) for binding to human OX40 or cynomolgus monkey OX40 than Pogalizumab, indicating they have relatively weaker binding affinity for OX40 than the reference antibody, which is probably related to the structural differences between the OX40 HCAb of the present invention and Pogalizumab.

TABLE 9
Binding affinity of antibodies to human OX40
protein and cynomolgus monkey OX40 protein

| | | Antigen | | | | |
|---|---|---|---|---|---|---|
| | Antigen | concentration | | | | |
| Antibody | protein | (nM) | KD (M) | kₒₙ (1/Ms) | k_{dis} (1/s) | Full R\hat{ }2 |
| PR002063 | Human | 200-6.25 | 1.30E−08 | 3.92E+04 | 5.11E−04 | 0.9987 |
| PR002065 | OX40 | 200-6.25 | 6.79E−09 | 4.96E+04 | 3.37E−04 | 0.9978 |
| PR002066 | | 200-6.25 | 8.71E−09 | 4.44E+04 | 3.87E−04 | 0.998 |
| PR002067 | | 200-6.25 | 4.16E−09 | 3.74E+04 | 1.56E−04 | 0.9991 |
| PR002077 | | 200-6.25 | 1.06E−08 | 3.43E+04 | 3.64E−04 | 0.9981 |
| Pogalizumab | | 25-1.56 | <1.0E−12 | 2.98E+05 | <1.0E−07 | 0.9978 |
| PR002063 | | 200-6.25 | 5.34E−08 | 3.60E+04 | 1.92E−03 | 0.9972 |
| PR002065 | Cynomolgus | 200-6.25 | 3.37E−08 | 3.08E+04 | 1.04E−03 | 0.9945 |
| PR002066 | monkey | 200-6.25 | 2.27E−08 | 4.27E+04 | 9.70E−04 | 0.9939 |
| PR002067 | OX40 | 200-6.25 | 1.92E−08 | 2.48E+04 | 4.75E−04 | 0.9945 |
| PR002077 | | 200-6.25 | 2.21E−08 | 3.27E+04 | 7.22E−04 | 0.9933 |
| Pogalizumab | | 25-1.56 | 8.94E−11 | 3.62E+05 | 3.24E−05 | 0.9981 |

### Example 8. Specific Binding of Antigen-Binding Proteins to OX40

OX40 belongs to the TNF tumor necrosis factor receptor superfamily, which consists of a large group of multifunctional receptors having the function of mediating immune and non-immune cells. Six receptors including CD40, OX40, 4-BB, CD27, GITR and CD30 have been identified as important immune co-stimulators. Similarly, the inducible T cell co-stimulatory factor (ICOS) is another receptor that plays a critical role in the function and survival of activated T cells or memory T cells.

This example is intended to investigate the specificity of in vitro binding of anti-human OX40 HCAb monoclonal antibodies by detecting 3 receptors of the TNF tumor necrosis factor receptor superfamily and ICOS by flow cytometry. The antibody binding assay at the cellular level was performed using a CHO-K1 cell strain overexpressing human OX40 (CHO-K1/hu OX40), a CHO-K1 cell strain overexpressing human CD40 (CHO-K1/hu CD40, Beijing KYinno, #KC-1286), a CHO-K1 cell strain overexpressing human 4-1BB (CHO-K1/hu 4-1BB, Genscript, #M00538) and an HEK293 cell strain overexpressing human ICOS (HEK293T/ICOS, Genscript, #KC-0210). Briefly, these cells were digested and resuspended in F12K or DMEM complete medium, and the cell density was adjusted to 1×10⁶ cells/mL. The cells were seeded in a 96-well V-bottom plate at 100 μL/well, and centrifuged, and then the supernatant was discarded. Then, the test antibodies diluted in a 3-fold gradient were added at 100 μL/well. The cells were incubated at 4° C. for 1 h away from light. Thereafter, the cells in each well were rinsed twice with 100 μL of pre-cooled PBS, and centrifuged at 500 g at 4° C. for 5 min, and then the supernatant was discarded. Then, 100 μL, of a fluorescent secondary antibody (Alexa Fluor 488-conjugated AffiniPure Goat Anti-Human IgG, Fcγ Fragment Specific, Jackson, #109-545-06, diluted in a 1:1000 ratio) was added to each well. The plate was incubated away from light at 4° C. for 30 min. The cells in each well were rinsed twice with 100 μL of pre-cooled PBS, and centrifuged at 500 g at 4° C. for 5 min, and then the supernatant was discarded. Finally, the cells in each well were resuspended in 200 μL of pre-cooled PBS, and the fluorescence signal values were read using NovoCyte flow cytometer (ACEA Biosciences).

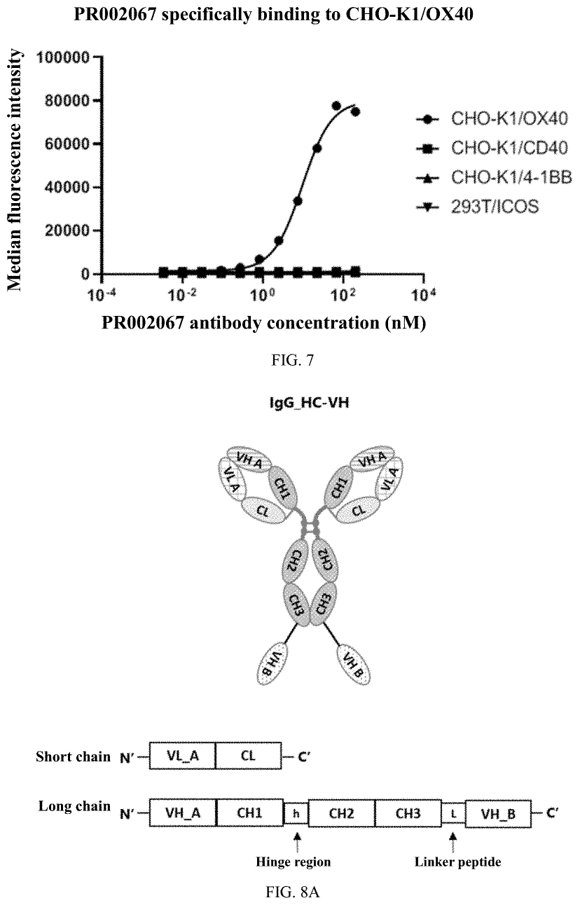

The results are shown in FIG. 7. It could be seen that PR002067 described herein specifically bound to CHO-K1/OX40 cells, but not to other members of the TNF tumor necrosis factor receptor superfamily.

### Example 9. Structure and Design of Bispecific Antibodies to OX40 and Tumor Targets

The anti-OX40 heavy-chain antibody and anti-PSMA (PR001331, H2L2 antibody, 202010096322.6), the anti-OX40 heavy-chain antibody and EPCAM (PRO01081, H2L2 antibody, 202010114063.5), the anti-OX40 heavy-chain antibody and CLDN18.2 (PR002726, H2L2 antibody, 201910941316.3), the anti-OX40 heavy-chain antibody and B7H4 (PR002408, H2L2 antibody), the anti-OX40 heavy-chain antibody and PD-L1 (PR000265, H2L2 antibody, 201910944996.4) selected from Example 1 to Example 8 were used to prepare bispecific antibodies capable of binding to two targets simultaneously, one end of which can recognize a tumor target TAA specifically expressed on the surface of tumor cells (e.g., PSMA, EPCAM, CLDN18.2, B7H4, or PD-L1), and the other end of which can bind to an OX40 molecule on T cells, and can recruit and activate T cells in the vicinity of tumor cells, thereby killing the tumor cells.

The information on the sequences of the H2L2 antibodies of the anti-tumor targets (e.g., PSMA, EPCAM, CLDN18.2, B7H4, and PD-L1) used in this example is shown in Table 10 below.

TABLE 10
Sequences of H2L2 antibodies of anti-tumor targets

| Antibody | Light | Heavy | | | | | | | | |
|---|---|---|---|---|---|---|---|---|---|---|
| No. | chain | chain | VL | VH | LCDR1 | LCDR2 | LCDR3 | HCDR1 | HCDR2 | HCDR3 |
| PR000265 | 265 | 205 | 199 | 139 | 111 | 119 | 129 | 10 | 39 | 79 |
| PR001081 | 266 | 206 | 200 | 140 | 112 | 120 | 130 | 11 | 40 | 80 |
| PR001331 | 267 | 207 | 201 | 141 | 113 | 121 | 131 | 12 | 41 | 81 |
| PR002408 | 268 | 231 | 202 | 165 | 114 | 122 | 132 | 17 | 51 | 91 |
| PR002726 | 269 | 232 | 203 | 166 | 112 | 120 | 133 | 18 | 52 | 92 |

The TAAxOX40 bispecific antibodies prepared in this example include a variety of molecular structures:

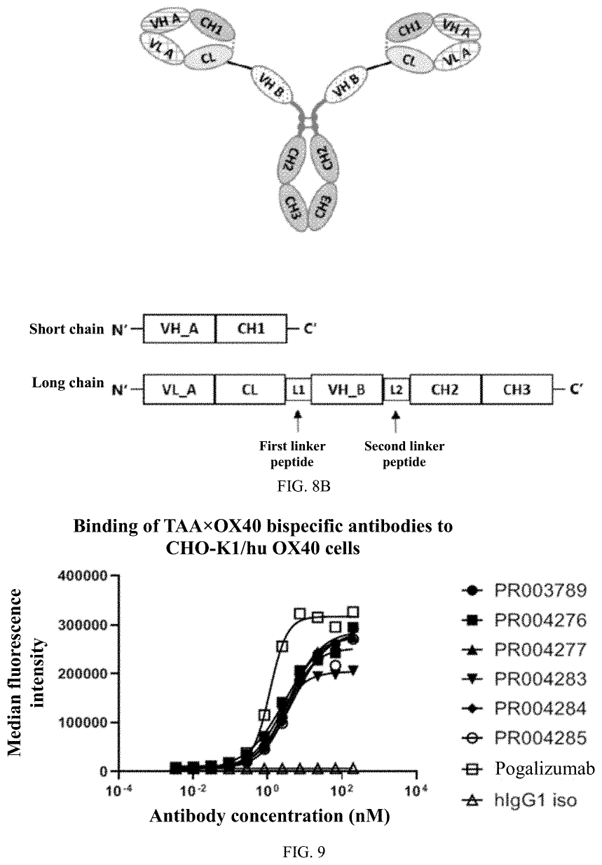

1) A molecule with an IgG-VH tetravalent symmetric structure (as shown in FIG. 8A), comprising two polypeptide chains: a polypeptide chain 1, also known as a short chain, comprising VL_A-CL from the amino-terminus to the carboxyl-terminus; and a polypeptide chain 2, also known as a long chain, comprising VH_A-CH1-h-CH2-CH3-L-VH_B from the amino-terminus to the carboxyl-terminus.

In one embodiment, CH3 is fusion-linked directly to VH_B in the polypeptide chain 2, i.e., L is 0 in length. In another embodiment, CH3 is linked to VH_B via a linker peptide L in the polypeptide chain 2; L may be the sequence listed in Table 11.

2) A molecule with a Fab-HCAb tetravalent symmetric structure (as shown in FIG. 8B), comprising two polypeptide chains: a polypeptide chain 1, also known as a short chain, comprising VH_A-CH1 from the amino-terminus to the carboxyl-terminus; and a polypeptide chain 2, also known as a long chain, comprising VL_A-CL-L1-VH_B-L2-CH2-CH3 from the amino-terminus to the carboxyl-terminus. In the structure, VL_A of the antibody A and VH_{_B} of the heavy-chain antibody B are fused on the same polypeptide chain, so that the mismatched byproducts generated by the association of VL_A and VH_B can be avoided.

VH_B is linked to CH2 via a linker peptide L2 in the polypeptide chain 2; L2 may be a hinge region or a hinge region-derived linker peptide sequence or the sequence listed in Table 11, preferably the sequence of human IgG1 hinge, human IgG1 hinge (C220S) or G5-LH.

In one embodiment, CL is fusion-linked directly to VH_B in the polypeptide chain 2, i.e., L1 is 0 in length. In another embodiment, CL is linked to VH_B via a linker peptide L1 in the polypeptide chain 2; and L1 may be the sequence listed in Table 11.

TABLE 11
Linker peptides

| Name of | Length of | | SEQ |
|---|---|---|---|
| linker | linker | Sequence of | ID |
| peptide | peptide | linker peptide | NO: |
| GS_4 | 4 | GSGS | 278 |
| GS_5 | 5 | GGGGS | 279 |
| GS_7 | 7 | GGGGSGS | 280 |
| GS_15 | 15 | GGGGSGGGGSGGGGS | 281 |
| GS_20 | 20 | GGGGSGGGGSGGGGS | 282 |
| | | GGGGS | |
| GS_25 | 25 | GGGGSGGGGSGGGGS | 283 |
| | | GGGGSGGGGS | |
| Human IgG1 | 15 | EPKSCDKTHTCPPCP | 284 |
| hinge | | | |
| Human IgG1 | 15 | EPKSSDKTHTCPPCP | 285 |
| hinge | | | |
| (C220S) | | | |
| H1_15 | 15 | EPKSSDKTHTPPPPP | 286 |
| G5-LH | 15 | GGGGGDKTHTCPPCP | 287 |
| H1_15-RT | 17 | EPKSSDKTHTPPPPPRT | 288 |
| L-GS_15-RT | 18 | LGGGGSGGGGSGGGGSRT | 289 |
| L-H1_15-RT | 18 | LEPKSSDKTHTPPPPPRT | 290 |
| KL-H1_15-RT | 19 | KLEPKSSDKTHTPPPPPRT | 291 |
| KL-H1_15-AS | 19 | KLEPKSSDKTHTPPPPPAS | 292 |
| RT-GS_5-KL | 9 | RTGGGGSKL | 293 |
| RT-GS_15-KL | 19 | RTGGGGSGGGGSGGGGSKL | 294 |
| RT-GS_25-KL | 29 | RTGGGGSGGGGSGGGGSGG | 295 |
| | | GGSGGGGSKL | |
| GS_2 | 2 | GS | |

Bispecific antibodies contain the Fc domain of IgG1 with mutations L234A and L235A or L234A and L235A and P329G (numbered according to the EU index).

The information on the bispecific antibodies with the IgG-VH tetravalent symmetric structure constructed in this example is shown in Table 12 below, the information on the bispecific antibodies with the Fab-HCAb tetravalent symmetric structure constructed in this example is shown in Table 13 below, and the physicochemical properties thereof are shown in Table 14 below.

TABLE 12
Bispecific antibodies with IgG-VH tetravalent symmetric structure

| Bispecific | | TAA | OX40 | Linker peptide | |
|---|---|---|---|---|---|
| antibody | Tumor-specific | antibody | antibody | (between VH_B | |
| molecule | target (TAA) | (IgG) | (VH_B) | and IgG) | Fc type (mutation) |
| PR003789 | PD-L1 | PR000265 | PR002067 | H1_15 | Human IgG1 |
| | | | | | (L234A, L235A, |
| | | | | | P329G) |
| PR004276 | B7H4 | PR002408 | PR002067 | H1_15-RT | Human IgG1 |
| | | | | | (L234A, L235A) |
| PR004283 | EPCAM | PR001081 | PR002067 | H1_15-RT | Human IgG1 |
| | | | | | (L234A, L235A) |
| PR004284 | PSMA | PR001331 | PR002067 | H1_15-RT | Human IgG1 |
| | | | | | (L234A, L235A) |
| PR004285 | CLDN18.2 | PR002726 | PR002067 | H1_15-RT | Human IgG1 |
| | | | | | (L234A, L235A) |

TABLE 13
Bispecific antibodies with Fab-HCAb tetravalent symmetric structure

| Bispecific | Tumor- | TAA | OX40 | First linker | Second linker | |
|---|---|---|---|---|---|---|
| antibody | specific | antibody | antibody | peptide (between | peptide (between | Fc type |
| molecule | target (TAA) | (Fab) | (VH_B) | Fab and VH_B) | VH_B and CH2) | (mutation) |
| PR004277 | B7H4 | PR002408 | PR002067 | H1_15 | Human IgG1 | Human IgG1 |
| | | | | | hinge | (L234A, |
| | | | | | (C220S) | L235A) |

TABLE 14
Expression and physicochemical properties of bispecific antibodies

| | | Plasmid | | |
|---|---|---|---|---|
| | | transfection | Yield | HPLC- |
| Bispecific | Expression | ratio (short | (mg/L) | SEC |
| antibody | system | chain:long | after first | purity |
| molecule | and volume | chain) | purification | (%) |
| PR003789 | HEK293-6E (40 ml) | 3:2 | 3.3 | 96.08 |
| PR004276 | HEK293-6E (40 ml) | 3:2 | 17.7 | 95.46 |
| PR004283 | HEK293-6E (40 ml) | 3:2 | 27.5 | 95.42 |
| PR004284 | HEK293-6E (40 ml) | 3:2 | 21.0 | 99.43 |
| PR004285 | HEK293-6E (40 ml) | 3:2 | 60.8 | 98.15 |
| PR004277 | HEK293-6E (40 ml) | 3:2 | 6.3 | 86.74 |

The information on the CDR numbers of the heavy chain and light chain sequences of the TAA×OX40 bispecific antibodies constructed in this example is shown in Table 15 below, and the information on the polypeptide chain numbers is shown in Table 16 below.

TABLE 15
CDR numbers of sequences of TAA × OX40 bispecific antibodies

| | Antigen- | | | | | | |
|---|---|---|---|---|---|---|---|
| Antibody | binding | | | | | | |
| No. | domain No. | LCDR1 | LCDR2 | LCDR3 | HCDR1 | HCDR2 | HCDR3 |
| PR003789 | #1 | 111 | 119 | 129 | 10 | 39 | 79 |
| | #2 | | | | 10 | 44 | 86 |
| PR004276 | #1 | 114 | 122 | 132 | 17 | 51 | 91 |
| | #2 | | | | 10 | 44 | 86 |
| PR004277 | #1 | 114 | 122 | 132 | 17 | 51 | 91 |
| | #2 | | | | 10 | 44 | 86 |
| PR004283 | #1 | 112 | 120 | 130 | 11 | 40 | 80 |
| | #2 | | | | 10 | 44 | 86 |
| PR004284 | #1 | 113 | 121 | 131 | 12 | 41 | 81 |
| | #2 | | | | 10 | 44 | 86 |
| PR004285 | #1 | 112 | 120 | 133 | 18 | 52 | 92 |
| | #2 | | | | 10 | 44 | 86 |

TABLE 16
Polypeptide chain numbers of TAA × OX40 bispecific antibodies

| | Polypeptide chain 1 | Polypeptide chain 2 |
|---|---|---|
| Antibody No. | SEQ ID NO | SEQ ID NO |
| PR003789 | 265 | 271 |
| PR004276 | 268 | 272 |
| PR004277 | 273 | 274 |
| PR004283 | 266 | 275 |
| PR004284 | 267 | 276 |
| PR004285 | 269 | 277 |

### Example 10. Binding Ability of Bispecific Antibodies to OX40 Cells as Assayed by FACS

This example is intended to investigate the in vitro binding activity of OX40×TAA bispecific antibodies obtained in Example 9 to human OX40. The antibody binding assay at the cellular level was performed using a CHO-K1 cell strain overexpressing human OX40 (CHO-K1/hu OX40). Briefly, CHO-K1/hu OX40 cells were digested, resuspended in an F12K complete medium, and washed with PBS, and the cell density was adjusted to 1×10⁶ cells/ml with PBS. The cells were seeded in a 96-well V-bottom plate (Corning, #3894) at 100 μL/well, followed by the addition of test antibodies diluted in a 3-fold gradient at concentrations that were 2 times the final concentrations at 100 μL/well. The cells were incubated at 4° C. for 1 h away from light. Thereafter, the cells in each well were rinsed twice with 100 μL of pre-cooled PBS, and centrifuged at 500 g at 4° C. for 5 min, and then the supernatant was discarded. Then, 100 μL, of a fluorescent secondary antibody (Alexa Fluor 488-conjugated AffiniPure Goat Anti-Human IgG, Fcγ Fragment Specific, Jackson, #109-545-06, diluted in a 1:1000 ratio) was added to each well. The plate was incubated away from light at 4° C. for 30 min. The cells in each well were rinsed twice with 100 μL of pre-cooled PBS, and centrifuged at 500 g at 4° C. for 5 min, and then the supernatant was discarded. Finally, the cells in each well were resuspended in 200 μL of pre-cooled PBS, and the fluorescence signal values were read using NovoCyte flow cytometer (ACEA Biosciences).

As shown in FIG. 9 and Table 17, the OX40×TAA bispecific antibodies of the present application were all able to bind to human OX40, with the detected binding ability increasing in a positive correlation with the antibody concentration. Their binding ability was comparable to that of the reference antibody Tab (Pogalizumab).

TABLE 17
Binding of OX40 × TAA bispecific
antibodies to human OX40 cells

| Antibody | EC50(nM) | Maximum MFI |
|---|---|---|
| PR003789 | 3.977 | 287338 |
| PR004276 | 2.799 | 281894 |
| PR004277 | 3.458 | 274396 |
| PR004283 | 1.809 | 204612 |
| PR004284 | 4.955 | 287445 |
| PR004285 | 3.548 | 252325 |
| Pogalizumab | 1.153 | 317098 |
| hIgG1 | ~70.97 | 6902 |

### Example 11. Binding Ability of Bispecific Antibodies to Corresponding Tumor-Associated Antigen Cells as Assayed by FACS

This example is intended to investigate the in vitro binding activity of OX40×TAA bispecific antibodies obtained in Example 9 to tumor-associated antigens (TAAs). The antibody binding assay at the cellular level was performed using SK-BR-3 (Cell Bank of Chinese Academy of Science, #TCHu225) highly expressing human B7H4, MDA-MB-231 (ATCC, HTB-26) highly expressing human PD-L1, LNCAP (Nanjing Cobioer, #CBP60346) highly expressing human PSMA, NUGC-4 (ExPASy, #CVCL_3082) highly expressing human CLDN18.2, or Capan-2 (ATCC, #HTB-80) highly expressing human EPCAM. Briefly, those cells were digested, resuspended in a complete medium, and washed with PBS, and the cell density was adjusted to 1×10⁶ cells/ml with PBS. The cells were seeded in a 96-well V-bottom plate (Corning, #3894) at 100 μL/well, and centrifuged, and then the supernatant was discarded. Then, the test antibodies diluted in a 3-fold gradient were added at 100 μL/well. The cells were incubated at 4° C. for 1 h away from light. Thereafter, the cells in each well were rinsed twice with 100 μL of pre-cooled PBS, and centrifuged at 500 g at 4° C. for 5 min, and then the supernatant was discarded. Then, 100 μL, of a fluorescent secondary antibody (Alexa Fluor 488-conjugated AffiniPure Goat Anti-Human IgG, Fcγ Fragment Specific, Jackson, #109-545-06, diluted in a 1:1000 ratio) was added to each well. The plate was incubated away from light at 4° C. for 30 min. The cells in each well were rinsed twice with 100 μL of pre-cooled PBS, and centrifuged at 500 g at 4° C. for 5 min, and then the supernatant was discarded. Finally, the cells in each well were resuspended in 200 μL of pre-cooled PBS, and the fluorescence signal values were read using NovoCyte flow cytometer (ACEA Biosciences).

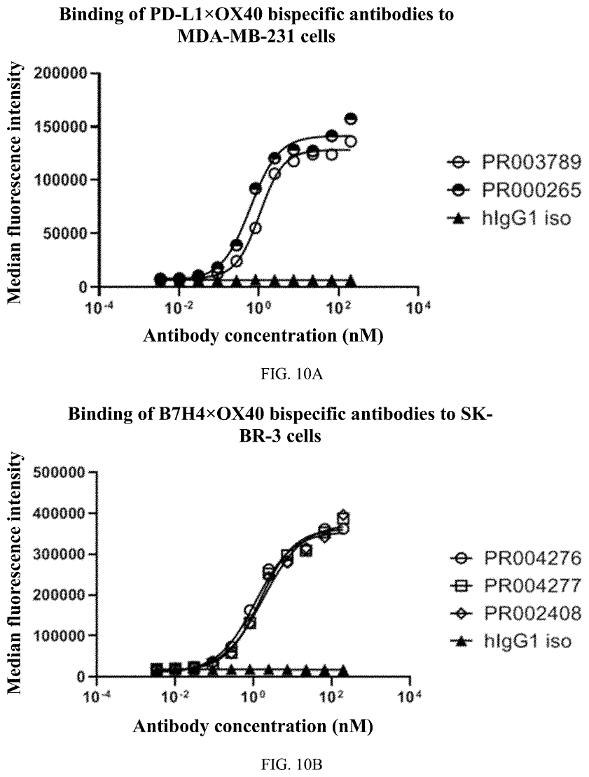

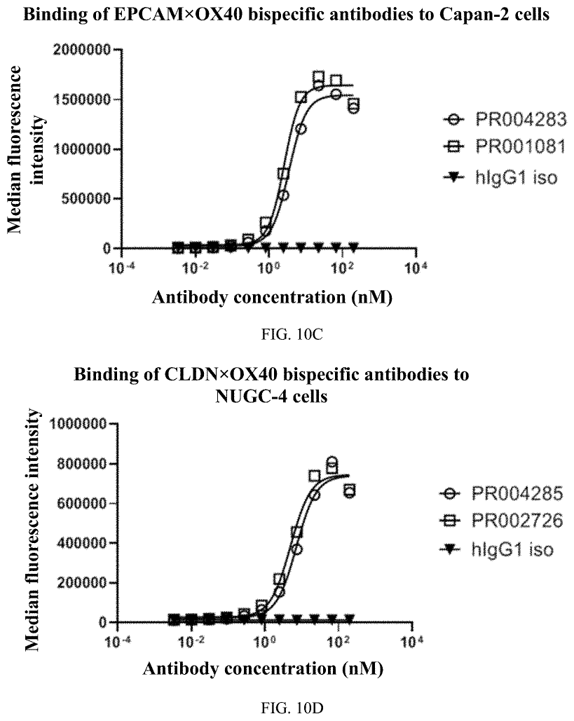

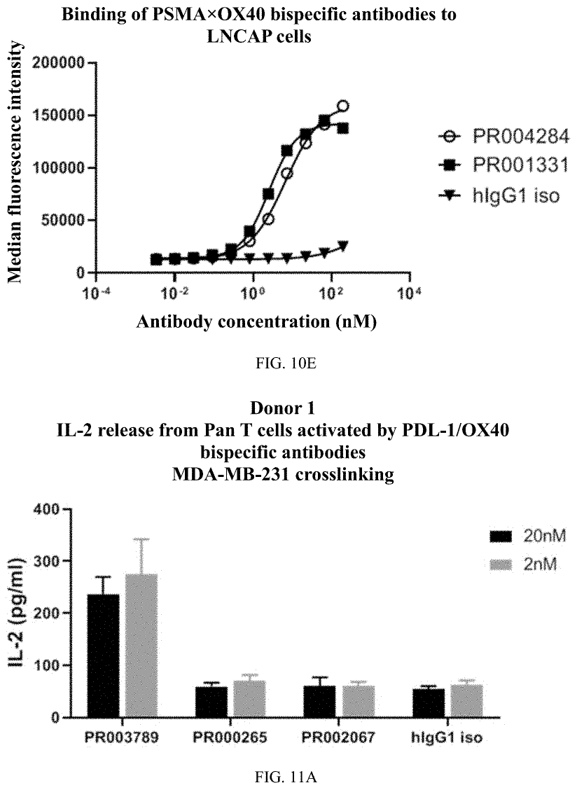

As shown in FIGS. 10A-10E and Tables 18-1, 18-2, 18-3, 18-4 and 18-5, the OX40×TAA bispecific antibodies of the present invention were all able to bind to the corresponding human tumor-associated antigens, with the detected binding ability increasing in a positive correlation with the antibody concentration. The bispecific antibodies exhibited comparable EC₅₀ and maximum values to the corresponding TAA parental monoclonal antibodies.

TABLE 18-1
Binding of OX40 × PD-L1 bispecific
antibodies to corresponding MDA-MB-231 cells

| | | Maximum median |
|---|---|---|
| Antibody | EC50(nM) | fluorescence intensity |
| PR003789 | 1.040 | 128390 |
| PR000265 | 0.6050 | 141594 |

TABLE 18-2
Binding of OX40 × B7H4 bispecific
antibodies to corresponding SK-BR-3 cells

| | | Maximum median |
|---|---|---|
| Antibody | EC50(nM) | fluorescence intensity |
| PR004276 | 1.139 | 354678 |
| PR004277 | 1.506 | 362253 |
| PR002408 | 1.813 | 373359 |

TABLE 18-3
Binding of OX40 × EpCAM bispecific
antibodies to corresponding Capan-2 cells

| | | Maximum median |
|---|---|---|
| Antibody | EC50(nM) | fluorescence intensity |
| PR004283 | 3.518 | 1544198 |
| PR001081 | 2.584 | 1642796 |

TABLE 18-4
Binding of OX40 × Claudin18.2 bispecific
antibodies to corresponding NUGC-4 cells

| | | Maximum median |
|---|---|---|
| Antibody | EC50(nM) | fluorescence intensity |
| PR004285 | 7.205 | 740185 |
| PR002726 | 4.982 | 745505 |

TABLE 18-5
Binding of OX40 × PSMA bispecific
antibodies to corresponding LNCAP cells

| | | Maximum median |
|---|---|---|
| Antibody | EC50(nM) | fluorescence intensity |
| PR004284 | 6.701 | 160857 |
| PR001331 | 2.578 | 142270 |
| PR000327 | 3.025 | 126604 |

### Example 12. In Vitro Activation of OX40 Pathway by Bispecific Antibodies

This example is intended to investigate the T cell activation activity of OX40×TAA bispecific antibodies by binding to the costimulatory molecule OX40 in the presence of target cells.

In this example, the target cells were cells expressing a particular antigen (e.g., a tumor-specific antigen), such as MDA-MB-231 (ATCC, HTB-26) highly expressing human PD-L1, or CHO-K1-hu B7H4 (constructed in house) highly expressing human B7H4, or HEK293-hu PSMA (Beijing KYinno, #KC-1005) highly expressing human PSMA, or Capan-2 (ATCC, HTB-80) highly expressing human EPCAM, or NUGC4 (JCRB, JCRB0834) highly expressing human CLDN18.2. The effector cells were isolated human T cells.

Specifically, 96-well flat-bottom plate (Corning, #3599) was coated firstly with 0.3 μg/mL anti-CD3 antibody OKT3 (Thermo, #16-0037-81) at 100 μL/well. Then, the density of human T cells (isolated from human PBMCs with a T cell isolation kit (Miltenyi, #130-096-535)) was adjusted to 2×10⁶ cells/mL, and the density of target cells was adjusted to 3×10⁵ cells/mL. The two cell suspensions were each seeded into a 96-well plate at 50 μL/well. Then, antibody molecules at different concentrations were added at 100 μL/well, and two duplicate wells were set for each concentration. hIgG1 iso (CrownBio, #C0001) and hIgG4 iso (CrownBio, #C0045) were used as a control. The 96-well plate was incubated in an incubator at 37° C. with 5% CO2 for 2 days. The supernatant after 48 h of culture was collected and the concentration of IL-2 in the supernatant was determined using an IL-2 ELISA kit (Thermo, #88-7025-88). The ELISA assay was performed by referring to the instructions of relevant kit. The absorbance values at 450 nm and 570 nm were read using a microplate reader (Molecular Devices, model: SpectraMax Plus), and the concentration of IL-2 was calculated from the standard readings (0D450-0D570). The data were processed and analyzed by plotting using GraphPad Prism 8 software.

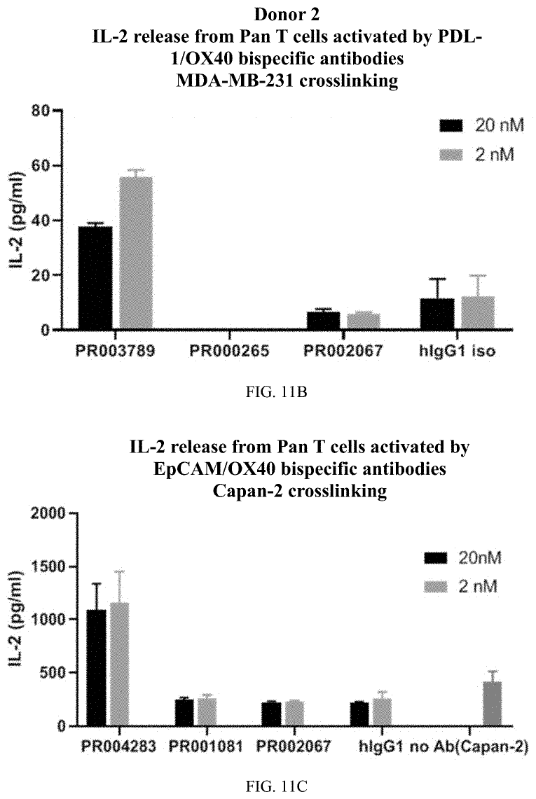

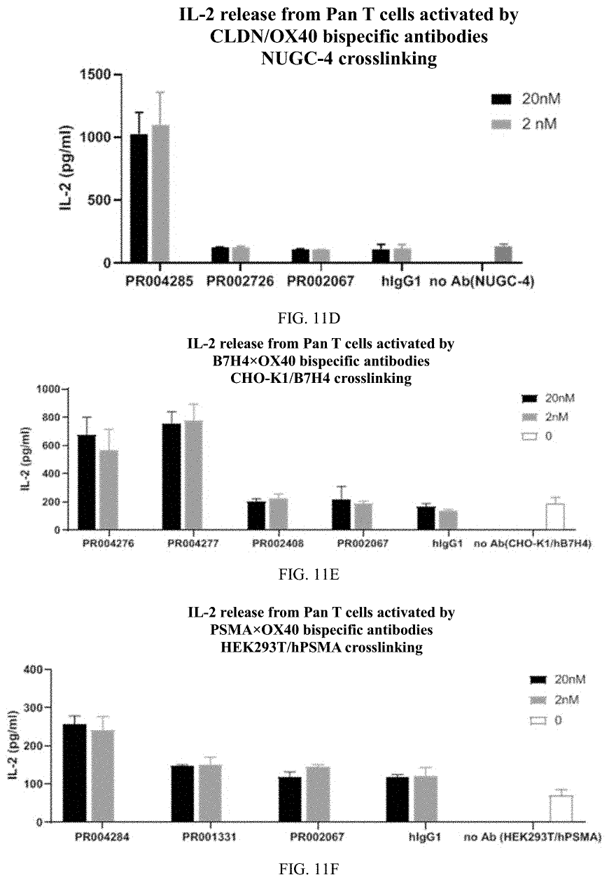

The results are shown in FIGS. 11A-11F (since effective results may not be obtained with PBMC donors alone due to individual differences, typically at least two donors were selected in parallel for one experiment, which were, in the examples, numbered as donor 1 and donor 2, respectively). It could be seen that the OX40/TAA bispecific antibodies described herein all had the activation of OX40-mediated T cell pathways under tumor cell crosslinking, indicating that OX40 bispecific antibodies are capable of specifically activating T cells in the presence of tumor target cells.

### Example 13. Heterologous Mixed Lymphocyte Reaction Assay (MLR)

This example is intended to investigate the T cell activation effect of PD-L1×OX40 bispecific antibody molecules by the mixed lymphocyte reaction (MLR).

In the first step, monocytes were isolated from PBMC cells (MT-Bio) of a first donor using CD14 magnetic beads (Meltenyi, #130-050-201) by referring to the instructions of the relevant kit. Then, 50 ng/mL of recombinant human IL-4 (PeproTech, #200-02-A) and 100 ng/mL of recombinant human GM-CSF (PeproTech, #300-03-A) were added, and after 6 days of induction at 37° C., immature dendritic cells (iDC cells) were obtained. 1 μg/mL lipopolysaccharide (LPS, Sigma, #L6529) was then added, and after 24 h of induction, mature dendritic cells (mDC cells) were obtained. In the second step, T lymphocytes were isolated from PBMC cells (MT-Bio) of a second donor using a T cell isolation kit (Meltenyi, #130-096-535). In the third step, the obtained T cells and mDC cells were seeded in a 96-well plate (T cells at 1×10⁵/well and mDC cells at 2×10⁴/well) at a ratio of 5:1. Then, antibody molecules at different concentrations were added at 50 μL/well, wherein the antibody concentration may be the final concentration of (10 nM, 1 nM), or a total of 8 concentrations obtained by a 3-fold gradient dilution from the highest final concentration of 50 nM; and two duplicate wells were set for each concentration. hIgG1 iso (CrownBio, #C0001) or a blank well was used as a control. The cells were incubated in an incubator at 37° C. with 5% CO2 for 5 days. In the fourth step, supernatants on day 3 and on day 5 were each collected. The IL-2 concentration in the 3-day supernatant was determined using an IL-2 ELISA kit (Thermo, #88-7025-77), and the IFN-γ concentration in the 5-day supernatant was determined using an IFN-γ ELISA kit (Thermo, #88-7316-88). The ELISA assay was performed by referring to the instructions of relevant kit. The absorbance values at 450 nm and 570 nm were read using a microplate reader (Molecular Devices, model: SpectraMax Plus), and the concentration of IL-2 or IFN-γ was calculated from the standard readings (0D450-0D570). The data were processed and analyzed by plotting using GraphPad Prism 8 software.

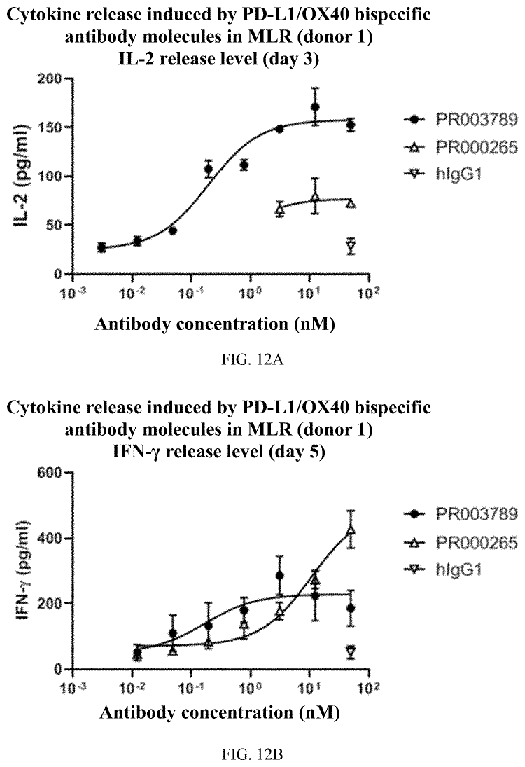

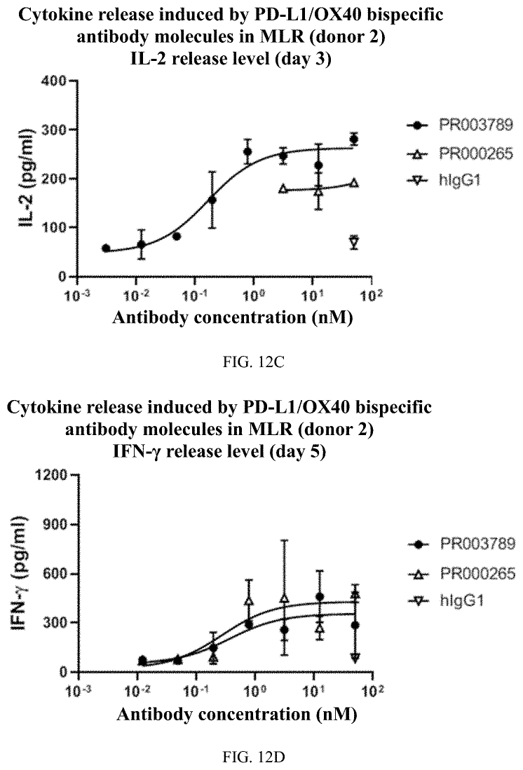

The results are shown in FIGS. 12A-12D and Tables 19-1 and 19-2. It could be seen that in two independent MLR experiments (different donor pairings), the anti-PD-L1 monoclonal antibody PR000265 had a more significant activation effect, but the PD-L1×OX40 bispecific antibody molecule PR003789 was able to further improve T cell function.

TABLE 19-1
Cytokine release induced by PD-L1 ×
OX40 bispecific antibodies in MLR (donor 1)

| | Donor 1 | | | | | |
|---|---|---|---|---|---|---|
| | IL-2 release | | | IFN-γ release | | |
| | | Maximum | Minimum | | Maximum | Minimum |
| Antibody | EC50(nM) | IL-2 | IL-2 | EC50(nM) | IFN-γ | IFN-γ |
| PR003789 | 0.1954 | 158 | 24.95 | 0.167 | 229.3 | 49.13 |
| PR000265 | | 77.26 | | 10.87 | 494.4 | 71.05 |

TABLE 19-2
Cytokine release induced by PD-L1 ×
OX40 bispecific antibodies in MLR (donor 2)

| | Donor 2 | | | | | |
|---|---|---|---|---|---|---|
| | IL-2 release | | | IFN-γ release | | |
| | | Maximum | Minimum | | Maximum | Minimum |
| Antibody | EC50(nM) | IL-2 | IL-2 | EC50(nM) | IFN-γ | IFN-γ |
| PR003789 | 0.1684 | 263.1 | 47.77 | 0.3694 | 356.5 | 50.71 |
| PR000265 | | | | 0.2765 | 429.7 | 22.83 |

### SUMMARY

In order to overcome the defects of the current OX40-targeted antibodies, the present invention obtains a class of fully human heavy-chain antibodies by immunizing Harbour HCAb mice. The antibodies of the present invention have activity in specifically binding to human OX40 and cynomolgus monkey OX40, and can promote greater activation of NF-κb, thereby stimulating the OX40 signaling pathway, and can activate the OX40 pathway in vitro and induce the activation of T cells, with the activation effect comparable to or greater than existing antibodies (e.g., Pogalizumab). Meanwhile, the antibodies or the antigen-binding fragments thereof of the present invention have crosslinking dependence of FcγRIIB (CD32B), which is one of the Fcγ receptor members.

The antibodies of the present invention are fully human “heavy chain”-only antibodies, which are only half the size of conventional IgG antibodies, and which, due to the absence of light chains, can be used for bispecific antibodies while solving the problems of light chain mismatch and heterodimerization. In the preparation of bispecific antibody molecules with the IgG-VH tetravalent symmetric structure and with the Fab-HCAb structure using the antibodies of the present invention and the H2L2 antibodies of the anti-tumor targets, the resulting bispecific antibodies are all capable of binding to human OX40 and the corresponding tumor-associated antigens, one end of which can recognize a tumor target TAA (e.g., PSMA, EPCAM, CLDN18.2, B7H4, or PD-L1) specifically expressed on the surface of tumor cells, and the other end of which can bind to an OX40 molecule on T cells, thereby specifically activating T cells in the tumor microenvironment, reducing toxicity caused by OX40 activation, and killing the tumor cells.

Although specific embodiments of the present invention have been described above, it will be appreciated by those skilled in the art that these embodiments are merely illustrative and that many changes or modifications can be made to these embodiments without departing from the principles and spirit of the present invention. The scope of protection of the present invention is therefore defined by the appended claims.

what is claimed is:

1. An OX40-targeted antibody or an antigen-binding fragment thereof, comprising a heavy chain variable region (VH),
wherein the VH comprises the following complementarity determining regions (CDRs) or mutations thereof: VH CDR1 with an amino acid sequence as set forth in SEQ ID NO: 10; VH CDR2 with an amino acid sequence as set forth in SEQ ID NO: 44; and VH CDR3 with an amino acid sequence as set forth in SEQ ID NO: 86, SEQ ID NO: 84 or SEQ ID NO: 89;
wherein the mutation is an insertion, deletion or substitution of 3, 2 or 1 amino acids on the basis of the amino acid sequences of the VH CDR1, VH CDR2 and VH CDR3 of the VH.

2. The OX40-targeted antibody or the antigen-binding fragment thereof according to claim 1, wherein the VH CDR1 has amino acid substitutions of F2L, T3S/P/I, S5D and/or S6D/C on the amino acid sequence as set forth in SEQ ID NO: 10, with an amino acid sequences as set forth in, for example, any one of SEQ ID NOs: 13-16 or SEQ ID NOs: 20-24; and/or
the VH CDR2 has 3,2 or 1 amino acid substitutions of S 1T, R3H/L/G/S, G4S, G5N/D and S6N/I/Q/T on the amino acid sequence as set forth in SEQ ID NO: 44, with an amino acid sequence as set forth in, for example, any one of SEQ ID NOs: 42-43, SEQ ID NOs: 45-50 or SEQ ID NOs: 54-60; and/or
the VH CDR3 has 2 or 1 amino acid substitutions of T2M/I/V, T5S, T6W/Y, D9C and Y10F/W on the amino acid sequence as set forth in SEQ ID NO: 86, with an amino acid sequence as set forth in, for example, any one of SEQ ID NOs: 82-83, SEQ ID NO: 85, SEQ ID NOs: 87-88, SEQ ID NO: 90 or SEQ ID NOs: 94-99.

3. The OX40-targeted antibody or the antigen-binding fragment thereof according to claim 1, comprising a heavy chain variable region (VH), wherein the VH comprises the following complementarity determining regions (CDRs) or mutations thereof: VH CDR1 with an amino acid sequence as set forth in any one of SEQ ID NO: 10, SEQ ID NOs: 13-16, or SEQ ID NOs: 20-24; VH CDR2 with an amino acid sequence as set forth in any one of SEQ ID NOs: 42-50 or SEQ ID NOs: 54-60; and VH CDR3 with an amino acid sequence as set forth in any one of SEQ ID NOs: 82-90 or SEQ ID NOs: 94-99;
preferably, the OX40-targeted antibody or the antigen-binding fragment thereof comprises a heavy chain variable region (VH), wherein,
the VH comprises VH CDR1, VH CDR2 and VH CDR3 with amino acid sequences as set forth in SEQ ID NOs: 13, 42 and 82, respectively; or,
the VH comprises VH CDR1, VH CDR2 and VH CDR3 with amino acid sequences as set forth in SEQ ID NOs: 10, 42 and 83, respectively; or,
the VH comprises VH CDR1, VH CDR2 and VH CDR3 with amino acid sequences as set forth in SEQ ID NOs: 14, 42 and 83, respectively; or,
the VH comprises VH CDR1, VH CDR2 and VH CDR3 with amino acid sequences as set forth in SEQ ID NOs: 10, 43 and 84, respectively; or,
the VH comprises VH CDR1, VH CDR2 and VH CDR3 with amino acid sequences as set forth in SEQ ID NOs: 13, 44 and 82, respectively; or,
the VH comprises VH CDR1, VH CDR2 and VH CDR3 with amino acid sequences as set forth in SEQ ID NOs: 15, 44 and 83, respectively; or,
the VH comprises VH CDR1, VH CDR2 and VH CDR3 with amino acid sequences as set forth in SEQ ID NOs: 10, 44 and 83, respectively; or,
the VH comprises VH CDR1, VH CDR2 and VH CDR3 with amino acid sequences as set forth in SEQ ID NOs: 10, 45 and 83, respectively; or,
the VH comprises VH CDR1, VH CDR2 and VH CDR3 with amino acid sequences as set forth in SEQ ID NOs: 10, 46 and 85, respectively; or,
the VH comprises VH CDR1, VH CDR2 and VH CDR3 with amino acid sequences as set forth in SEQ ID NOs: 10, 44 and 86, respectively; or,
the VH comprises VH CDR1, VH CDR2 and VH CDR3 with amino acid sequences as set forth in SEQ ID NOs: 16, 42 and 82, respectively; or,
the VH comprises VH CDR1, VH CDR2 and VH CDR3 with amino acid sequences as set forth in SEQ ID NOs: 10, 47 and 87, respectively; or,
the VH comprises VH CDR1, VH CDR2 and VH CDR3 with amino acid sequences as set forth in SEQ ID NOs: 16, 44 and 83, respectively; or,
the VH comprises VH CDR1, VH CDR2 and VH CDR3 with amino acid sequences as set forth in SEQ ID NOs: 10, 45 and 88, respectively; or,
the VH comprises VH CDR1, VH CDR2 and VH CDR3 with amino acid sequences as set forth in SEQ ID NOs: 10, 48 and 89, respectively; or,
the VH comprises VH CDR1, VH CDR2 and VH CDR3 with amino acid sequences as set forth in SEQ ID NOs: 10, 49 and 90, respectively; or,
the VH comprises VH CDR1, VH CDR2 and VH CDR3 with amino acid sequences as set forth in SEQ ID NOs: 10, 49 and 83, respectively; or,
the VH comprises VH CDR1, VH CDR2 and VH CDR3 with amino acid sequences as set forth in SEQ ID NOs: 10, 50 and 90, respectively; or,
the VH comprises VH CDR1, VH CDR2 and VH CDR3 with amino acid sequences as set forth in SEQ ID NOs: 20, 44 and 86, respectively; or,
the VH comprises VH CDR1, VH CDR2 and VH CDR3 with amino acid sequences as set forth in SEQ ID NOs: 21, 44 and 86, respectively; or,
the VH comprises VH CDR1, VH CDR2 and VH CDR3 with amino acid sequences as set forth in SEQ ID NOs: 10, 54 and 86, respectively; or,
the VH comprises VH CDR1, VH CDR2 and VH CDR3 with amino acid sequences as set forth in SEQ ID NOs: 10, 55 and 86, respectively; or,
the VH comprises VH CDR1, VH CDR2 and VH CDR3 with amino acid sequences as set forth in SEQ ID NOs: 10, 56 and 86, respectively; or,
the VH comprises VH CDR1, VH CDR2 and VH CDR3 with amino acid sequences as set forth in SEQ ID NOs: 10, 42 and 86, respectively; or,
the VH comprises VH CDR1, VH CDR2 and VH CDR3 with amino acid sequences as set forth in SEQ ID NOs: 10, 57 and 86, respectively; or,
the VH comprises VH CDR1, VH CDR2 and VH CDR3 with amino acid sequences as set forth in SEQ ID NOs: 10, 58 and 86, respectively; or,
the VH comprises VH CDR1, VH CDR2 and VH CDR3 with amino acid sequences as set forth in SEQ ID NOs: 10, 59 and 86, respectively; or,
the VH comprises VH CDR1, VH CDR2 and VH CDR3 with amino acid sequences as set forth in SEQ ID NOs: 10, 60 and 86, respectively; or,
the VH comprises VH CDR1, VH CDR2 and VH CDR3 with amino acid sequences as set forth in SEQ ID NOs: 10, 44 and 94, respectively; or,
the VH comprises VH CDR1, VH CDR2 and VH CDR3 with amino acid sequences as set forth in SEQ ID NOs: 10, 44 and 95, respectively; or,
the VH comprises VH CDR1, VH CDR2 and VH CDR3 with amino acid sequences as set forth in SEQ ID NOs: 10, 44 and 96, respectively; or,
the VH comprises VH CDR1, VH CDR2 and VH CDR3 with amino acid sequences as set forth in SEQ ID NOs: 10, 44 and 97, respectively; or,
the VH comprises VH CDR1, VH CDR2 and VH CDR3 with amino acid sequences as set forth in SEQ ID NOs: 10, 44 and 98, respectively; or,
the VH comprises VH CDR1, VH CDR2 and VH CDR3 with amino acid sequences as set forth in SEQ ID NOs: 10, 44 and 99, respectively; or,
the VH comprises VH CDR1, VH CDR2 and VH CDR3 with amino acid sequences as set forth in SEQ ID NOs: 10, 54 and 95, respectively; or,
the VH comprises VH CDR1, VH CDR2 and VH CDR3 with amino acid sequences as set forth in SEQ ID NOs: 10, 54 and 97, respectively; or,
the VH comprises VH CDR1, VH CDR2 and VH CDR3 with amino acid sequences as set forth in SEQ ID NOs: 10, 55 and 95, respectively; or,
the VH comprises VH CDR1, VH CDR2 and VH CDR3 with amino acid sequences as set forth in SEQ ID NOs: 10, 55 and 97, respectively; or,
the VH comprises VH CDR1, VH CDR2 and VH CDR3 with amino acid sequences as set forth in SEQ ID NOs: 10, 56 and 95, respectively; or,
the VH comprises VH CDR1, VH CDR2 and VH CDR3 with amino acid sequences as set forth in SEQ ID NOs: 10, 56 and 97, respectively; or,
the VH comprises VH CDR1, VH CDR2 and VH CDR3 with amino acid sequences as set forth in SEQ ID NOs: 22, 54 and 86, respectively; or,
the VH comprises VH CDR1, VH CDR2 and VH CDR3 with amino acid sequences as set forth in SEQ ID NOs: 23, 54 and 86, respectively; or,
the VH comprises VH CDR1, VH CDR2 and VH CDR3 with amino acid sequences as set forth in SEQ ID NOs: 24, 54 and 86, respectively; or,
the VH comprises VH CDR1, VH CDR2 and VH CDR3 with amino acid sequences as set forth in SEQ ID NOs: 21, 54 and 86, respectively; or,
the VH comprises VH CDR1, VH CDR2 and VH CDR3 with amino acid sequences as set forth in SEQ ID NOs: 10, 54 and 99, respectively; or,
the VH comprises VH CDR1, VH CDR2 and VH CDR3 with amino acid sequences as set forth in SEQ ID NOs: 22, 54 and 99, respectively; or,
the VH comprises VH CDR1, VH CDR2 and VH CDR3 with amino acid sequences as set forth in SEQ ID NOs: 23, 54 and 99, respectively; or,
the VH comprises VH CDR1, VH CDR2 and VH CDR3 with amino acid sequences as set forth in SEQ ID NOs: 24, 54 and 99, respectively; or,
the VH comprises VH CDR1, VH CDR2 and VH CDR3 with amino acid sequences as set forth in SEQ ID NOs: 21, 54 and 99, respectively;
preferably, the VH comprises an amino acid sequence as set forth in any one of SEQ ID NOs: 142-164 or SEQ ID NOs: 168-198;
more preferably, the OX40-targeted antibody or the antigen-binding fragment thereof further comprises a heavy chain constant region Fc domain of a human antibody, wherein the heavy chain constant region Fc domain of the human antibody comprises a heavy chain constant region Fc domain of human IgG1, IgG2, IgG3, or IgG4;
even more preferably, the OX40-targeted antibody or the antigen-binding fragment thereof comprises one polypeptide chain, wherein the polypeptide chain comprises amino acid sequences as set forth in any one of SEQ ID NOs: 208-230 or SEQ ID NOs: 234-264.

4. The OX40-targeted antibody or the antigen-binding fragment thereof according to claim 1, including IgG, Fab, Fab′, F(ab′)₂, Fv, scFv, HCAb, VH, bispecific antibodies, multispecific antibodies, single-domain antibodies, or any other antibodies retaining part of the ability of an antibody to specifically bind to an antigen, or monoclonal or polyclonal antibodies prepared from the above antibodies.

5. A bispecific binding protein comprising at least two protein functional regions: a protein functional region A and a protein functional region B, wherein the protein functional region A and the protein functional region B target different antigens, wherein the protein functional region B targets OX40 and the protein functional region A targets a non-OX40 antigen; the protein functional region B is selected from the OX40-targeted antibody or the antigen-binding fragment thereof according to claim 1.

6. The bispecific binding protein according to claim 5, wherein the protein functional region A targets PD-L1, B7H4, PSMA, EPCAM or CLDN18.2.

7. The bispecific binding protein according to claim 6, wherein the protein functional region A is a PD-L1 antibody or an antigen-binding fragment thereof, a B7H4 antibody or an antigen-binding fragment thereof, a PSMA antibody or an antigen-binding fragment thereof, an EPCAM antibody or an antigen-binding fragment thereof, or a CLDN18.2 antibody or an antigen-binding fragment thereof;
preferably:
the PD-L1 antibody or the antigen-binding fragment thereof comprises a light chain variable region (VL) and a heavy chain variable region (VH), wherein the VL comprises VL CDR1, VL CDR2 and VL CDR3 with amino acid sequences as set forth in SEQ ID NOs: 111, 119 and 129, respectively, and the VH comprises VH CDR1, VH CDR2 and VH CDR3 with amino acid sequences as set forth in SEQ ID NOs: 10, 39 and 79, respectively; or,
the EPCAM antibody or the antigen-binding fragment thereof comprises a light chain variable region (VL) and a heavy chain variable region (VH), wherein the VL comprises VL CDR1, VL CDR2 and VL CDR3 with amino acid sequences as set forth in SEQ ID NOs: 112, 120 and 130, respectively, and the VH comprises VH CDR1, VH CDR2 and VH CDR3 with amino acid sequences as set forth in SEQ ID NOs: 11, 40 and 80, respectively; or,
the PSMA antibody or the antigen-binding fragment thereof comprises a light chain variable region (VL) and a heavy chain variable region (VH), wherein the VL comprises VL CDR1, VL CDR2 and VL CDR3 with amino acid sequences as set forth in SEQ ID NOs: 113, 121 and 131, respectively, and the VH comprises VH CDR1, VH CDR2 and VH CDR3 with amino acid sequences as set forth in SEQ ID NOs: 12, 41 and 81, respectively; or,
the B7H4 antibody or the antigen-binding fragment thereof comprises a light chain variable region (VL) and a heavy chain variable region (VH), wherein the VL comprises VL CDR1, VL CDR2 and VL CDR3 with amino acid sequences as set forth in SEQ ID NOs: 114, 122 and 132, respectively, and the VH comprises VH CDR1, VH CDR2 and VH CDR3 with amino acid sequences as set forth in SEQ ID NOs: 17, 51 and 91, respectively; or,
the CLDN18.2 antibody or the antigen-binding fragment thereof comprises a light chain variable region (VL) and a heavy chain variable region (VH), wherein the VL comprises VL CDR1, VL CDR2 and VL CDR3 with amino acid sequences as set forth in SEQ ID NOs: 112, 120 and 133, respectively, and the VH comprises VH CDR1, VH CDR2 and VH CDR3 with amino acid sequences as set forth in SEQ ID NOs: 18, 52 and 92, respectively.

8. The bispecific binding protein according to claim 5, wherein the protein functional region A comprises a light chain variable region (VL) and a heavy chain variable region (VH), wherein the VL comprises VL CDR1, VL CDR2 and VL CDR3 with amino acid sequences as set forth in SEQ ID NOs: 111, 119 and 129, respectively, and the VH comprises VH CDR1, VH CDR2 and VH CDR3 with amino acid sequences as set forth in SEQ ID NOs: 10, 39 and 79, respectively; and the protein functional region B comprises a heavy chain variable region (VH), wherein the VH comprises VH CDR1, VH CDR2 and VH CDR3 with amino acid sequences as set forth in SEQ ID NOs: 10, 44 and 86, respectively; or,
the protein functional region A comprises a light chain variable region (VL) and a heavy chain variable region (VH), wherein the VL comprises VL CDR1, VL CDR2 and VL CDR3 with amino acid sequences as set forth in SEQ ID NOs: 114, 122 and 132, respectively, and the VH comprises VH CDR1, VH CDR2 and VH CDR3 with amino acid sequences as set forth in SEQ ID NOs: 17, 51 and 91, respectively; and the protein functional region B comprises a heavy chain variable region (VH), wherein the VH comprises VH CDR1, VH CDR2 and VH CDR3 with amino acid sequences as set forth in SEQ ID NOs: 10, 44 and 86, respectively; or,
the protein functional region A comprises a light chain variable region (VL) and a heavy chain variable region (VH), wherein the VL comprises VL CDR1, VL CDR2 and VL CDR3 with amino acid sequences as set forth in SEQ ID NOs: 112, 120 and 130, respectively, and the VH comprises VH CDR1, VH CDR2 and VH CDR3 with amino acid sequences as set forth in SEQ ID NOs: 11, 40 and 80, respectively; and the protein functional region B comprises a heavy chain variable region (VH), wherein the VH comprises VH CDR1, VH CDR2 and VH CDR3 with amino acid sequences as set forth in SEQ ID NOs: 10, 44 and 86, respectively; or,
the protein functional region A comprises a light chain variable region (VL) and a heavy chain variable region (VH), wherein the VL comprises VL CDR1, VL CDR2 and VL CDR3 with amino acid sequences as set forth in SEQ ID NOs: 113, 121 and 131, respectively, and the VH comprises VH CDR1, VH CDR2 and VH CDR3 with amino acid sequences as set forth in SEQ ID NOs: 12, 41 and 81, respectively; and the protein functional region B comprises a heavy chain variable region (VH), wherein the VH comprises VH CDR1, VH CDR2 and VH CDR3 with amino acid sequences as set forth in SEQ ID NOs: 10, 44 and 86, respectively; or,
the protein functional region A comprises a light chain variable region (VL) and a heavy chain variable region (VH), wherein the VL comprises VL CDR1, VL CDR2 and VL CDR3 with amino acid sequences as set forth in SEQ ID NOs: 112, 120 and 133, respectively, and the VH comprises VH CDR1, VH CDR2 and VH CDR3 with amino acid sequences as set forth in SEQ ID NOs: 18, 52 and 92, respectively; and the protein functional region B comprises a heavy chain variable region (VH), wherein the VH comprises VH CDR1, VH CDR2 and VH CDR3 with amino acid sequences as set forth in SEQ ID NOs: 10, 44 and 86, respectively.

9. The bispecific binding protein according to claim 5, wherein
the protein functional region A comprises a light chain variable region (VL) and a heavy chain variable region (VH), wherein the VL comprises an amino acid sequence as set forth in SEQ ID NO: 199, and the VH comprises an amino acid sequence as set forth in SEQ ID NO: 139; and the protein functional region B comprises a heavy chain variable region, wherein the VH comprises an amino acid sequence as set forth in SEQ ID NO: 154; or,
the protein functional region A comprises a light chain variable region (VL) and a heavy chain variable region (VH), wherein the VL comprises an amino acid sequence as set forth in SEQ ID NO: 202, and the VH comprises an amino acid sequence as set forth in SEQ ID NO: 165; and the protein functional region B comprises a heavy chain variable region (VH), wherein the VH comprises an amino acid sequence as set forth in SEQ ID NO: 154; or,
the protein functional region A comprises a light chain variable region (VL) and a heavy chain variable region (VH), wherein the VL comprises an amino acid sequence as set forth in SEQ ID NO: 200, and the VH comprises an amino acid sequence as set forth in SEQ ID NO: 140; and the protein functional region B comprises a heavy chain variable region (VH), wherein the VH comprises an amino acid sequence as set forth in SEQ ID NO: 154; or,
the protein functional region A comprises a light chain variable region (VL) and a heavy chain variable region (VH), wherein the VL comprises an amino acid sequence as set forth in SEQ ID NO: 201, and the VH comprises an amino acid sequence as set forth in SEQ ID NO: 141; and the protein functional region B comprises a heavy chain variable region (VH), wherein the VH comprises an amino acid sequence as set forth in SEQ ID NO: 154; or,
the protein functional region A comprises a light chain variable region (VL) and a heavy chain variable region (VH), wherein the VL comprises an amino acid sequence as set forth in SEQ ID NO: 203, and the VH comprises an amino acid sequence as set forth in SEQ ID NO: 166; and the protein functional region B comprises a heavy chain variable region (VH), wherein the VH comprises an amino acid sequence as set forth in SEQ ID NO: 154.

10. The bispecific binding protein according to claim 5, wherein the protein functional region A and/or the protein functional region B is in the form of IgG, Fab, Fab′, F(ab′)₂, Fv, scFv, VH or HCAb, wherein the protein functional region A and the protein functional region B are not both IgG;
preferably, the number of the Fab, Fab′, F(ab′)2, Fv, scFv or VH is one or more than one.

11. The bispecific binding protein according to claim 5, wherein the protein functional region B is of a single VH structure, and the protein functional region A is of an IgG structure; and the protein functional region B is preferably linked to the C-terminus of the protein functional region A;
preferably, the bispecific binding protein comprises a first polypeptide chain and a second polypeptide chain, wherein the first polypeptide chain is as shown in formula: N′-VL_{_A}-CL-C′, and the second polypeptide chain is as shown in formula: N′-VH_{_A}-CH1-h-CH2-CH3-L-VH_{_B}-C′;
wherein the VH_{_B} is VH of the protein functional region B, the VL_{_A} and the VH_{_A} are VL and VH of the protein functional region A, respectively, the h is a hinge region, and the L is a linker peptide;
more preferably, the L is preferably 0 in length or has an amino acid sequence as set forth in any one of SEQ ID NOs: 278-295 or an amino acid sequence of GS.

12. The bispecific binding protein according to claim 5, wherein the protein functional region B is of an HCAb structure, and the protein functional region A is of a Fab structure; and the protein functional region B is preferably linked to the C-terminus of the protein functional region A;
preferably:
the bispecific binding protein comprises a first polypeptide chain and a second polypeptide chain, wherein the first polypeptide chain is as shown in formula: N′-VH_{_A}-CH1-C′, and the second polypeptide chain is as shown in formula: N′-VL_{_A}-CL-L1-VH_{_B}-L2-CH2-CH3-C′;
or, the bispecific binding protein comprises a first polypeptide chain and a second polypeptide chain, wherein the first polypeptide chain is as shown in formula: N′-VL_{_A}-CL-C′, and the second polypeptide chain is as shown in formula: N′-VH_{_A}-CH1-L1-VH_{_B}-L2-CH2-CH3-C′;
wherein the VH_{_B} is VH of the protein functional region B, the VL_{_A} and the VH_{_A} are VL and VH of the protein functional region A, respectively, and the L1 and L2 are linker peptides;
more preferably, the L1 or L2 is preferably 0 in length or has an amino acid sequence as set forth in any one of SEQ ID NOs: 278-295 or an amino acid sequence of GS, for example, the L1 has an amino acid sequence as set forth in SEQ ID NO: 286, and the L2 has an amino acid sequence as set forth in SEQ ID NO: 285.

13. The bispecific binding protein according to claim 5, comprising a first polypeptide chain and a second polypeptide chain, wherein,
the first polypeptide chain comprises an amino acid sequence as set forth in SEQ ID NO: 265; and the second polypeptide chain comprises an amino acid sequence as set forth in SEQ ID NO: 271; or,
the first polypeptide chain comprises an amino acid sequence as set forth in SEQ ID NO: 268; and the second polypeptide chain comprises an amino acid sequence as set forth in SEQ ID NO: 272; or,
the first polypeptide chain comprises an amino acid sequence as set forth in SEQ ID NO: 273; and the second polypeptide chain comprises an amino acid sequence as set forth in SEQ ID NO: 274; or,
the first polypeptide chain comprises an amino acid sequence as set forth in SEQ ID NO: 266; and the second polypeptide chain comprises an amino acid sequence as set forth in SEQ ID NO: 275; or,
the first polypeptide chain comprises an amino acid sequence as set forth in SEQ ID NO: 267; and the second polypeptide chain comprises an amino acid sequence as set forth in SEQ ID NO: 276; or,
the first polypeptide chain comprises an amino acid sequence as set forth in SEQ ID NO: 269; and the second polypeptide chain comprises an amino acid sequence as set forth in SEQ ID NO: 277.

14. A chimeric antigen receptor comprising the OX40-targeted antibody or the antigen-binding fragment thereof according to claim 1 or a bispecific binding protein;
wherein the bispecific binding protein comprising at least two protein functional regions: a protein functional region A and a protein functional region B, wherein the protein functional region A and the protein functional region B target different antigens, wherein the protein functional region B targets OX40 and the protein functional region A targets a non-OX40 antigen; the protein functional region B is selected from the OX40-targeted antibody or the antigen-binding fragment thereof according to claim 1.

15. An immune cell comprising the chimeric antigen receptor according to claim 14.

16. An isolated nucleic acid encoding the OX40-targeted antibody or the antigen-binding fragment thereof according to claim 1 or a bispecific binding protein;
wherein the bispecific binding protein comprising at least two protein functional regions: a protein functional region A and a protein functional region B, wherein the protein functional region A and the protein functional region B target different antigens, wherein the protein functional region B targets OX40 and the protein functional region A targets a non-OX40 antigen; the protein functional region B is selected from the OX40-targeted antibody or the antigen-binding fragment thereof according to claim 1.

17. A recombinant expression vector comprising the isolated nucleic acid according to claim 16, wherein preferably, the expression vector comprises a eukaryotic cell expression vector and/or a prokaryotic cell expression vector.

18. A transformant comprising the isolated nucleic acid according to claim 16, wherein
preferably, the transformant has a host cell being a prokaryotic cell and/or a eukaryotic cell, wherein the prokaryotic cell is preferably an E. coli cell such as TG1 and BL21, and the eukaryotic cell is preferably an HEK293 cell or a CHO cell.

19. A method for preparing an OX40-targeted antibody or an antigen-binding fragment thereof, or a bispecific binding protein, comprising culturing the transformant according to claim 18, and obtaining the OX40-targeted antibody or the antigen-binding fragment thereof, or the bispecific binding protein from a culture.

20. An antibody-drug conjugate comprising an antibody moiety
and a conjugate moiety, wherein the antibody moiety comprises the OX40-targeted antibody or the antigen-binding fragment thereof according to claim 1 and/or the a bispecific binding protein, the conjugate moiety includes, but is not limited to, a detectable label, a drug, a toxin, a cytokine, a radionuclide, an enzyme, or a combination thereof, and the antibody moiety and the conjugate moiety are conjugated via a chemical bond or a linker;
wherein, the bispecific binding protein comprising at least two protein functional regions: a protein functional region A and a protein functional region B, wherein the protein functional region A and the protein functional region B target different antigens, wherein the protein functional region B targets OX40 and the protein functional region A targets a non-OX40 antigen; the protein functional region B is selected from the OX40-targeted antibody or the antigen-binding fragment thereof according to claim 1.

21. A pharmaceutical composition comprising the OX40-targeted antibody or the antigen-binding fragment thereof according to claim 1, a bispecific binding protein, and a pharmaceutically acceptable carrier, wherein
the bispecific binding protein comprising at least two protein functional regions: a protein functional region A and a protein functional region B, wherein the protein functional region A and the protein functional region B target different antigens, wherein the protein functional region B targets OX40 and the protein functional region A targets a non-OX40 antigen; the protein functional region B is selected from the OX40-targeted antibody or the antigen-binding fragment thereof according to claim 1;
preferably, the pharmaceutical composition further comprises an additional anti-tumor antibody as an active ingredient.

22. (canceled)

23. A method for detecting OX40 in a sample, comprising detecting with the OX40-targeted antibody or the antigen-binding fragment thereof according to claim 1 and/or a bispecific binding protein, wherein
the bispecific binding protein comprising at least two protein functional regions: a protein functional region A and a protein functional region B, wherein the protein functional region A and the protein functional region B target different antigens, wherein the protein functional region B targets OX40 and the protein functional region A targets a non-OX40 antigen; the protein functional region B is selected from the OX40-targeted antibody or the antigen-binding fragment thereof according to claim 1;
preferably, the method is for non-diagnostic purposes.

24. A kit comprising the OX40-targeted antibody or the antigen-binding fragment thereof according to claim 1, a bispecific binding protein and optionally instructions
wherein the bispecific binding protein comprising at least two protein functional regions: a protein functional region A and a protein functional region B, wherein the protein functional region A and the protein functional region B target different antigens, wherein the protein functional region B targets OX40 and the protein functional region A targets a non-OX40 antigen; the protein functional region B is selected from the OX40-targeted antibody or the antigen-binding fragment thereof according to claim 1.

25. (canceled)

26. A method for diagnosis, preventing and/or treating a tumor in a subject in need of, comprising administering an effective amount of the OX40-targeted antibody or the antigen-binding fragment thereof according to claim 1 to the subject.


SEQUENCE LISTING (295 sequences)

SEQ ID NO: 1 - protein, 25 aa, Artificial Sequence [sequence omitted]

SEQ ID NO: 2 - protein, 25 aa, Artificial Sequence
  PR001081 HFWR1 Chothia, PR002408 HFWR1 Chothia
QVQLVESGGGVVQPGRSLRLSCAAS

SEQ ID NO: 3 - protein, 25 aa, Artificial Sequence
  PR001331 HFWR1 Chothia
QVQLVESGGGVVQPGRSLRLSCSAS

SEQ ID NO: 4 - protein, 25 aa, Artificial Sequence
  PR002055 HFWR1 Chothia, PR002056 HFWR1 Chothia, PR002057 HFWR1 Chothia
EVQLLESGGGLVQPGGSLRLSCAAS

SEQ ID NO: 5 - protein, 25 aa, Artificial Sequence
  PR002059 HFWR1 Chothia
EVQLLESGGGLVQPGGSLRLSCVVS

SEQ ID NO: 6 - protein, 25 aa, Artificial Sequence
  PR002064 HFWR1 Chothia
EVQLVETGGGLVQPGGSLRLSCAAS

SEQ ID NO: 7 - protein, 25 aa, Artificial Sequence
  PR002066 HFWR1 Chothia, PR002070 HFWR1 Chothia
EVQLLESGGGLVQPGGSLRLSCIAS

SEQ ID NO: 8 - protein, 25 aa, Artificial Sequence
  PR002075 HFWR1 Chothia
EVQLVETGGGLIQPGGSLRLSCAAS

SEQ ID NO: 9 - protein, 25 aa, Artificial Sequence
  PR003475 HFWR1 Chothia
EVQLVQSGAEVKKPGASVKVSCKAS

SEQ ID NO: 10 - protein, 7 aa, Artificial Sequence
  PR000265 HCDR1 Chothia, PR002056 HCDR1 Chothia, PR002058 HCDR1 Chothia
GFTFSSY

SEQ ID NO: 11 - protein, 7 aa, Artificial Sequence
  PR001081 HCDR1 Chothia
GFTFSRY

SEQ ID NO: 12 - protein, 7 aa, Artificial Sequence
  PR001331 HCDR1 Chothia
GFTFINY

SEQ ID NO: 13 - protein, 7 aa, Artificial Sequence
  PR002055 HCDR1 Chothia, PR002059 HCDR1 Chothia
GLTFSSY

SEQ ID NO: 14 - protein, 7 aa, Artificial Sequence
  PR002057 HCDR1 Chothia
GFIFSSY

SEQ ID NO: 15 - protein, 7 aa, Artificial Sequence
  PR002060 HCDR1 Chothia
GFTFSDY

SEQ ID NO: 16 - protein, 7 aa, Artificial Sequence
  PR002068 HCDR1 Chothia, PR002070 HCDR1 Chothia
GFSFSSY

SEQ ID NO: 17 - protein, 7 aa, Artificial Sequence
  PR002408 HCDR1 Chothia
GFTFRSF

SEQ ID NO: 18 - protein, 7 aa, Artificial Sequence
  PR002726 HCDR1 Chothia
GFTFSSF

SEQ ID NO: 19 - protein, 7 aa, Artificial Sequence
  PR003475 HCDR1 Chothia
GYTFTDS

SEQ ID NO: 20 - protein, 7 aa, Artificial Sequence
  PR005362 HCDR1 Chothia
GLPFDCY

SEQ ID NO: 21 - protein, 7 aa, Artificial Sequence
  PR005363 HCDR1 Chothia, PR005387 HCDR1 Chothia, PR005392 HCDR1 Chothia
GLPFDSY

SEQ ID NO: 22 - protein, 7 aa, Artificial Sequence
  PR005384 HCDR1 Chothia, PR005389 HCDR1 Chothia
GLPFSSY

SEQ ID NO: 23 - protein, 7 aa, Artificial Sequence
  PR005385 HCDR1 Chothia, PR005390 HCDR1 Chothia
GLTFDSY

SEQ ID NO: 24 - protein, 7 aa, Artificial Sequence
  PR005386 HCDR1 Chothia, PR005391 HCDR1 Chothia
GFPFDSY

SEQ ID NO: 25 - protein, 19 aa, Artificial Sequence
  PR000265 HFWR2 Chothia
WMSWVRQAPGKGLEWVANI

SEQ ID NO: 26 - protein, 19 aa, Artificial Sequence
  PR001081 HFWR2 Chothia
DMNWVRQAPGKGLEWVAVI

SEQ ID NO: 27 - protein, 19 aa, Artificial Sequence
  PR001331 HFWR2 Chothia
GMNWVRQAPGKGLEWVAVI

SEQ ID NO: 28 - protein, 19 aa, Artificial Sequence
  PR002055 HFWR2 Chothia
AMSWVRQAPGKGLEWISAI

SEQ ID NO: 29 - protein, 19 aa, Artificial Sequence
  PR002056 HFWR2 Chothia, PR002057 HFWR2 Chothia, PR002058 HFWR2 Chothia
AMSWVRQAPGKGLEWVSAI

SEQ ID NO: 30 - protein, 19 aa, Artificial Sequence
  PR002059 HFWR2 Chothia, PR002069 HFWR2 Chothia
AMSWVRQAPGKGREWVSAI

SEQ ID NO: 31 - protein, 19 aa, Artificial Sequence
  PR002062 HFWR2 Chothia, PR002064 HFWR2 Chothia
AMSWVRQAPGKGQEWVSAI

SEQ ID NO: 32 - protein, 19 aa, Artificial Sequence
  PR002063 HFWR2 Chothia
AMSWVRQAPGRGLEWVSAI

SEQ ID NO: 33 - protein, 19 aa, Artificial Sequence
  PR002068 HFWR2 Chothia
AMTWVRQAPGKGLEWVSAI

SEQ ID NO: 34 - protein, 19 aa, Artificial Sequence
  PR002072 HFWR2 Chothia
AMSWVRQASGKGLEWISAI

SEQ ID NO: 35 - protein, 19 aa, Artificial Sequence
  PR002074 HFWR2 Chothia
AMSWVRQAPGKGLEWVSGI

SEQ ID NO: 36 - protein, 19 aa, Artificial Sequence
  PR002408 HFWR2 Chothia
GMHWVRQAPGKGLEWVAVI

SEQ ID NO: 37 - protein, 19 aa, Artificial Sequence
  PR002726 HFWR2 Chothia
VMSWVRQAPGKGLEWVSTI

SEQ ID NO: 38 - protein, 19 aa, Artificial Sequence
  PR003475 HFWR2 Chothia
YMSWVRQAPGQGLEWIGDM

SEQ ID NO: 39 - protein, 6 aa, Artificial Sequence
  PR000265 HCDR2 Chothia
KQEGSE

SEQ ID NO: 40 - protein, 6 aa, Artificial Sequence
  PR001081 HCDR2 Chothia
WFDGSN

SEQ ID NO: 41 - protein, 6 aa, Artificial Sequence
  PR001331 HCDR2 Chothia
WYDGSN

SEQ ID NO: 42 - protein, 6 aa, Artificial Sequence
  PR002055 HCDR2 Chothia, PR002056 HCDR2 Chothia, PR002057 HCDR2 Chothia
SGGGGS

SEQ ID NO: 43 - protein, 6 aa, Artificial Sequence
  PR002058 HCDR2 Chothia, PR002073 HCDR2 Chothia
SGGSGS

SEQ ID NO: 44 - protein, 6 aa, Artificial Sequence
  PR002059 HCDR2 Chothia, PR002060 HCDR2 Chothia, PR002061 HCDR2 Chothia
SGRGGS

SEQ ID NO: 45 - protein, 6 aa, Artificial Sequence
  PR002062 HCDR2 Chothia, PR002064 HCDR2 Chothia, PR002072 HCDR2 Chothia
SGSGGS

SEQ ID NO: 46 - protein, 6 aa, Artificial Sequence
  PR002065 HCDR2 Chothia
SGRGDI

SEQ ID NO: 47 - protein, 6 aa, Artificial Sequence
  PR002069 HCDR2 Chothia
SGGGGN

SEQ ID NO: 48 - protein, 6 aa, Artificial Sequence
  PR002074 HCDR2 Chothia
SGGGNN

SEQ ID NO: 49 - protein, 6 aa, Artificial Sequence
  PR002075 HCDR2 Chothia, PR002076 HCDR2 Chothia
SGSGGN

SEQ ID NO: 50 - protein, 6 aa, Artificial Sequence
  PR002077 HCDR2 Chothia
SGRGNI

SEQ ID NO: 51 - protein, 6 aa, Artificial Sequence
  PR002408 HCDR2 Chothia
SYDASN

SEQ ID NO: 52 - protein, 6 aa, Artificial Sequence
  PR002726 HCDR2 Chothia
SGSGRS

SEQ ID NO: 53 - protein, 6 aa, Artificial Sequence
  PR003475 HCDR2 Chothia
YPDNGD

SEQ ID NO: 54 - protein, 6 aa, Artificial Sequence
  PR005364 HCDR2 Chothia, PR005378 HCDR2 Chothia, PR005379 HCDR2 Chothia
SGRGGQ

SEQ ID NO: 55 - protein, 6 aa, Artificial Sequence
  PR005365 HCDR2 Chothia, PR005380 HCDR2 Chothia, PR005381 HCDR2 Chothia
SGLGGQ

SEQ ID NO: 56 - protein, 6 aa, Artificial Sequence
  PR005366 HCDR2 Chothia, PR005382 HCDR2 Chothia, PR005383 HCDR2 Chothia
SGRSGQ

SEQ ID NO: 57 - protein, 6 aa, Artificial Sequence
  PR005368 HCDR2 Chothia
SGLGGS

SEQ ID NO: 58 - protein, 6 aa, Artificial Sequence
  PR005369 HCDR2 Chothia
TGRGGQ

SEQ ID NO: 59 - protein, 6 aa, Artificial Sequence
  PR005370 HCDR2 Chothia
SGHGGT

SEQ ID NO: 60 - protein, 6 aa, Artificial Sequence
  PR005371 HCDR2 Chothia
SGRGGT

SEQ ID NO: 61 - protein, 41 aa, Artificial Sequence
  PR000265 HFWR3 Chothia
KYYVDSVKGRFTISRDNAKNSLYLQMNSLRAEDTAVYYCAR

SEQ ID NO: 62 - protein, 41 aa, Artificial Sequence
  PR001081 HFWR3 Chothia
KYYADSVKGRFAISRDNSKNTLYLQMNSLRAEDTAVYYCAR

SEQ ID NO: 63 - protein, 41 aa, Artificial Sequence
  PR001331 HFWR3 Chothia
KYYADSVRGRFTISRDNSKNTLYLQMNSLRAEDTAVYYCAR

SEQ ID NO: 64 - protein, 41 aa, Artificial Sequence
  PR002055 HFWR3 Chothia, PR002061 HFWR3 Chothia, PR002075 HFWR3 Chothia
TYYADSVRGRFTISRDNSKNTLYLQMNSLRAEDTAVYYCAN

SEQ ID NO: 65 - protein, 41 aa, Artificial Sequence
  PR002056 HFWR3 Chothia, PR002068 HFWR3 Chothia, PR002071 HFWR3 Chothia
IFYADSVRGRFTISRDNSKNTLYLQMNSLRAEDTAVYYCAN

SEQ ID NO: 66 - protein, 41 aa, Artificial Sequence
  PR002057 HFWR3 Chothia, PR002058 HFWR3 Chothia, PR002073 HFWR3 Chothia
TFYADSVRGRFTISRDNSKNTLYLQMNSLRAEDTAVYYCAD

SEQ ID NO: 67 - protein, 41 aa, Artificial Sequence
  PR002059 HFWR3 Chothia, PR002066 HFWR3 Chothia, PR002069 HFWR3 Chothia
TFYADSVRGRFTISRDNSKNTLYLQMNSLRAEDTAVYYCAN

SEQ ID NO: 68 - protein, 41 aa, Artificial Sequence
  PR002060 HFWR3 Chothia
TYYADSVRGRFTISRDNSKNTLYLQMTSLRAEDTAVYYCAN

SEQ ID NO: 69 - protein, 41 aa, Artificial Sequence
  PR002062 HFWR3 Chothia, PR002063 HFWR3 Chothia, PR002064 HFWR3 Chothia
TFYADSVRGRFTISRDNSKNTLYLQMSSLRAEDTAVYYCAN

SEQ ID NO: 70 - protein, 41 aa, Artificial Sequence
  PR002065 HFWR3 Chothia
TFYADSVRGRFTISRDNSKNTLYLQMNSLRAEDTALYYCAN

SEQ ID NO: 71 - protein, 41 aa, Artificial Sequence
  PR002067 HFWR3 Chothia, PR005362 HFWR3 Chothia, PR005363 HFWR3 Chothia
TFYADSVRGRFTISRDNSKNTLYLQMNSLRAEDTAMYYCVD

SEQ ID NO: 72 - protein, 41 aa, Artificial Sequence
  PR002070 HFWR3 Chothia
TFYADSVRGRFNISRDNSKNTLYLQMNSLRVEDTAVYYCAN

SEQ ID NO: 73 - protein, 41 aa, Artificial Sequence
  PR002072 HFWR3 Chothia
TYYADSVRGRFTISRDNSKNTLYLQMNSLRADDTAVYYCAN

SEQ ID NO: 74 - protein, 41 aa, Artificial Sequence
  PR002074 HFWR3 Chothia
TFYADSVRGRFTISRDNSKNTLYLQMNSLRAEDTAVYYCMR

SEQ ID NO: 75 - protein, 41 aa, Artificial Sequence
  PR002408 HFWR3 Chothia
EYYADSVKGRFIISRDNSKDTLYLQMNSLRAGDTAVYYCAK

SEQ ID NO: 76 - protein, 41 aa, Artificial Sequence
  PR002726 HFWR3 Chothia
TYYADSVKGRFTISRDNSKNTLHLQMNSLRAEDTAVYYCAK

SEQ ID NO: 77 - protein, 41 aa, Artificial Sequence
  PR003475 HFWR3 Chothia
SSYNQKFRERVTITRDTSTSTAYLELSSLRSEDTAVYYCVL

SEQ ID NO: 78 - protein, 41 aa, Artificial Sequence
  PR005364 HFWR3 Chothia, PR005365 HFWR3 Chothia, PR005366 HFWR3 Chothia
IFYADSVRGRFTISRDNSKNTLYLQMNSLRAEDTAMYYCVD

SEQ ID NO: 79 - protein, 10 aa, Artificial Sequence
  PR000265 HCDR3 Chothia
DRAVAGAFDI

SEQ ID NO: 80 - protein, 9 aa, Artificial Sequence
  PR001081 HCDR3 Chothia
EMAAAGFYL

SEQ ID NO: 81 - protein, 13 aa, Artificial Sequence
  PR001331 HCDR3 Chothia
GPGYGGHSDAFDI

SEQ ID NO: 82 - protein, 10 aa, Artificial Sequence
  PR002055 HCDR3 Chothia, PR002059 HCDR3 Chothia, PR002068 HCDR3 Chothia
GMTGSTDVDY

SEQ ID NO: 83 - protein, 10 aa, Artificial Sequence
  PR002056 HCDR3 Chothia, PR002057 HCDR3 Chothia, PR002060 HCDR3 Chothia
GMTGTTDVDY

SEQ ID NO: 84 - protein, 9 aa, Artificial Sequence
  PR002058 HCDR3 Chothia, PR002073 HCDR3 Chothia
GVTGTDFDF

SEQ ID NO: 85 - protein, 10 aa, Artificial Sequence
  PR002065 HCDR3 Chothia
GMTGSTDVDF

SEQ ID NO: 86 - protein, 10 aa, Artificial Sequence
  PR002067 HCDR3 Chothia, PR005362 HCDR3 Chothia, PR005363 HCDR3 Chothia
GTTGTTDVDY

SEQ ID NO: 87 - protein, 10 aa, Artificial Sequence
  PR002069 HCDR3 Chothia
GVTGTTDVDY

SEQ ID NO: 88 - protein, 10 aa, Artificial Sequence
  PR002072 HCDR3 Chothia
GMTGTTDVDF

SEQ ID NO: 89 - protein, 9 aa, Artificial Sequence
  PR002074 HCDR3 Chothia
GWELPLLEN

SEQ ID NO: 90 - protein, 10 aa, Artificial Sequence
  PR002075 HCDR3 Chothia, PR002077 HCDR3 Chothia
GITGTTDVDY

SEQ ID NO: 91 - protein, 10 aa, Artificial Sequence
  PR002408 HCDR3 Chothia
GGGLRWYFAY

SEQ ID NO: 92 - protein, 11 aa, Artificial Sequence
  PR002726 HCDR3 Chothia
DAAAAGTKFDY

SEQ ID NO: 93 - protein, 8 aa, Artificial Sequence
  PR003475 HCDR3 Chothia
APRWYFSV

SEQ ID NO: 94 - protein, 10 aa, Artificial Sequence
  PR005372 HCDR3 Chothia
GTTGSWDVCY

SEQ ID NO: 95 - protein, 10 aa, Artificial Sequence
  PR005373 HCDR3 Chothia, PR005378 HCDR3 Chothia, PR005380 HCDR3 Chothia
GTTGTWDVDW

SEQ ID NO: 96 - protein, 10 aa, Artificial Sequence
  PR005374 HCDR3 Chothia
GTTGSWDVDW

SEQ ID NO: 97 - protein, 10 aa, Artificial Sequence
  PR005375 HCDR3 Chothia, PR005379 HCDR3 Chothia, PR005381 HCDR3 Chothia
GTTGSYDVDW

SEQ ID NO: 98 - protein, 10 aa, Artificial Sequence
  PR005376 HCDR3 Chothia
GTTGSTDVDW

SEQ ID NO: 99 - protein, 10 aa, Artificial Sequence
  PR005377 HCDR3 Chothia, PR005388 HCDR3 Chothia, PR005389 HCDR3 Chothia
GTTGTWDVDY

SEQ ID NO: 100 - protein, 11 aa, Artificial Sequence
  PR000265 HFWR4 Chothia, PR001331 HFWR4 Chothia
WGQGTMVTVSS

SEQ ID NO: 101 - protein, 11 aa, Artificial Sequence
  PR001081 HFWR4 Chothia
WGRGTLVTVSS

SEQ ID NO: 102 - protein, 11 aa, Artificial Sequence
  PR002055 HFWR4 Chothia, PR002056 HFWR4 Chothia, PR002057 HFWR4 Chothia
WGQGTLVTVSS

SEQ ID NO: 103 - protein, 11 aa, Artificial Sequence
  PR002063 HFWR4 Chothia, PR002075 HFWR4 Chothia, PR002076 HFWR4 Chothia
WGQGTRVTVSS

SEQ ID NO: 104 - protein, 11 aa, Artificial Sequence
  PR002066 HFWR4 Chothia, PR002068 HFWR4 Chothia
WGQGTQVTVSS

SEQ ID NO: 105 - protein, 11 aa, Artificial Sequence
  PR002072 HFWR4 Chothia
WGQGTPVTVSS

SEQ ID NO: 106 - protein, 23 aa, Artificial Sequence
  PR000265 LFWR1 Chothia
DIQMTQSPSTLSASVGDRVTVTC

SEQ ID NO: 107 - protein, 23 aa, Artificial Sequence
  PR001081 LFWR1 Chothia, PR002726 LFWR1 Chothia
EIVLTQSPATLSLSPGERATLSC

SEQ ID NO: 108 - protein, 23 aa, Artificial Sequence
  PR001331 LFWR1 Chothia
DIQMTQSPSSLSASIGDRVTITC

SEQ ID NO: 109 - protein, 23 aa, Artificial Sequence
  PR002408 LFWR1 Chothia
EIVMTQSPATLSVSPGERATLSC

SEQ ID NO: 110 - protein, 23 aa, Artificial Sequence
  PR003475 LFWR1 Chothia
DIQMTQSPSSLSASVGDRVTITC

SEQ ID NO: 111 - protein, 11 aa, Artificial Sequence
  PR000265 LCDR1 Chothia
RASQSIYIWLA

SEQ ID NO: 112 - protein, 11 aa, Artificial Sequence
  PR001081 LCDR1 Chothia, PR002726 LCDR1 Chothia
RASQSVSSYLA

SEQ ID NO: 113 - protein, 11 aa, Artificial Sequence
  PR001331 LCDR1 Chothia
RASQSINSYLN

SEQ ID NO: 114 - protein, 11 aa, Artificial Sequence
  PR002408 LCDR1 Chothia
RASQSISSNLG

SEQ ID NO: 115 - protein, 11 aa, Artificial Sequence
  PR003475 LCDR1 Chothia
RASQDISNYLN

SEQ ID NO: 116 - protein, 15 aa, Artificial Sequence
  PR000265 LFWR2 Chothia
WYQQKPGKAPNLLIY

SEQ ID NO: 117 - protein, 15 aa, Artificial Sequence
  PR001081 LFWR2 Chothia, PR002408 LFWR2 Chothia, PR002726 LFWR2 Chothia
WYQQKPGQAPRLLIY

SEQ ID NO: 118 - protein, 15 aa, Artificial Sequence
  PR001331 LFWR2 Chothia, PR003475 LFWR2 Chothia
WYQQKPGKAPKLLIY

SEQ ID NO: 119 - protein, 7 aa, Artificial Sequence
  PR000265 LCDR2 Chothia
KASSLET

SEQ ID NO: 120 - protein, 7 aa, Artificial Sequence
  PR001081 LCDR2 Chothia, PR002726 LCDR2 Chothia
DASNRAT

SEQ ID NO: 121 - protein, 7 aa, Artificial Sequence
  PR001331 LCDR2 Chothia
TASSLLS

SEQ ID NO: 122 - protein, 7 aa, Artificial Sequence
  PR002408 LCDR2 Chothia
GASTRAT

SEQ ID NO: 123 - protein, 7 aa, Artificial Sequence
  PR003475 LCDR2 Chothia
YTSRLRS

SEQ ID NO: 124 - protein, 32 aa, Artificial Sequence
  PR000265 LFWR3 Chothia
GVPSRFSGSGSGTEFTLTISSLQPDDFATYYC

SEQ ID NO: 125 - protein, 32 aa, Artificial Sequence
  PR001081 LFWR3 Chothia, PR002726 LFWR3 Chothia
GIPARFSGSGSGTDFTLTISSLEPEDFAVYYC

SEQ ID NO: 126 - protein, 32 aa, Artificial Sequence
  PR001331 LFWR3 Chothia
GVPSRFSASGSWTDFSLTISSLQPEDFATYYC

SEQ ID NO: 127 - protein, 32 aa, Artificial Sequence
  PR002408 LFWR3 Chothia
GIPARFSGSGSGTEFTLTISSLQSEDFAVYYC

SEQ ID NO: 128 - protein, 32 aa, Artificial Sequence
  PR003475 LFWR3 Chothia
GVPSRFSGSGSGTDFTLTISSLQPEDFATYYC

SEQ ID NO: 129 - protein, 9 aa, Artificial Sequence
  PR000265 LCDR3 Chothia
QQYYGSSRT

SEQ ID NO: 130 - protein, 10 aa, Artificial Sequence
  PR001081 LCDR3 Chothia
QQRSNWPPIT

SEQ ID NO: 131 - protein, 9 aa, Artificial Sequence
  PR001331 LCDR3 Chothia
QQSFSTPYT

SEQ ID NO: 132 - protein, 10 aa, Artificial Sequence
  PR002408 LCDR3 Chothia
QQYQSWPPLT

SEQ ID NO: 133 - protein, 9 aa, Artificial Sequence
  PR002726 LCDR3 Chothia
QQRSNWPLT

SEQ ID NO: 134 - protein, 9 aa, Artificial Sequence
  PR003475 LCDR3 Chothia
QQGHTLPPT

SEQ ID NO: 135 - protein, 10 aa, Artificial Sequence
  PR000265 LFWR4 Chothia, PR003475 LFWR4 Chothia
FGQGTKVEIK

SEQ ID NO: 136 - protein, 10 aa, Artificial Sequence
  PR001081 LFWR4 Chothia
FGQGTRLEIK

SEQ ID NO: 137 - protein, 10 aa, Artificial Sequence
  PR001331 LFWR4 Chothia
FGQGTNLEIK

SEQ ID NO: 138 - protein, 10 aa, Artificial Sequence
  PR002408 LFWR4 Chothia, PR002726 LFWR4 Chothia
FGGGTKVEIK

SEQ ID NO: 139 - protein, 119 aa, Artificial Sequence
  PR000265 VH
EVQLVESGGGLVQPGGSLRLSCAASGFTFSSYWMSWVRQAPGKGLEWVANIKQEGSEKYYVDSVKGRFTISRDNAKNSLYLQMNSLRAEDTAVYYCARDRAVAGAFDIWGQGTMVTVSS

SEQ ID NO: 140 - protein, 118 aa, Artificial Sequence
  PR001081 VH
QVQLVESGGGVVQPGRSLRLSCAASGFTFSRYDMNWVRQAPGKGLEWVAVIWFDGSNKYYADSVKGRFAISRDNSKNTLYLQMNSLRAEDTAVYYCAREMAAAGFYLWGRGTLVTVSS

SEQ ID NO: 141 - protein, 122 aa, Artificial Sequence
  PR001331 VH
QVQLVESGGGVVQPGRSLRLSCSASGFTFINYGMNWVRQAPGKGLEWVAVIWYDGSNKYYADSVRGRFTISRDNSKNTLYLQMNSLRAEDTAVYYCARGPGYGGHSDAFDIWGQGTMVTVSS

SEQ ID NO: 142 - protein, 119 aa, Artificial Sequence
  PR002055 VH
EVQLLESGGGLVQPGGSLRLSCAASGLTFSSYAMSWVRQAPGKGLEWISAISGGGGSTYYADSVRGRFTISRDNSKNTLYLQMNSLRAEDTAVYYCANGMTGSTDVDYWGQGTLVTVSS

SEQ ID NO: 143 - protein, 119 aa, Artificial Sequence
  PR002056 VH
EVQLLESGGGLVQPGGSLRLSCAASGFTFSSYAMSWVRQAPGKGLEWVSAISGGGGSIFYADSVRGRFTISRDNSKNTLYLQMNSLRAEDTAVYYCANGMTGTTDVDYWGQGTLVTVSS

SEQ ID NO: 144 - protein, 119 aa, Artificial Sequence
  PR002057 VH
EVQLLESGGGLVQPGGSLRLSCAASGFIFSSYAMSWVRQAPGKGLEWVSAISGGGGSTFYADSVRGRFTISRDNSKNTLYLQMNSLRAEDTAVYYCADGMTGTTDVDYWGQGTLVTVSS

SEQ ID NO: 145 - protein, 118 aa, Artificial Sequence
  PR002058 VH
EVQLLESGGGLVQPGGSLRLSCAASGFTFSSYAMSWVRQAPGKGLEWVSAISGGSGSTFYADSVRGRFTISRDNSKNTLYLQMNSLRAEDTAVYYCADGVTGTDFDFWGQGTLVTVSS

SEQ ID NO: 146 - protein, 119 aa, Artificial Sequence
  PR002059 VH
EVQLLESGGGLVQPGGSLRLSCVVSGLTFSSYAMSWVRQAPGKGREWVSAISGRGGSTFYADSVRGRFTISRDNSKNTLYLQMNSLRAEDTAVYYCANGMTGSTDVDYWGQGTLVTVSS

SEQ ID NO: 147 - protein, 119 aa, Artificial Sequence
  PR002060 VH
EVQLLESGGGLVQPGGSLRLSCAASGFTFSDYAMSWVRQAPGKGLEWVSAISGRGGSTYYADSVRGRFTISRDNSKNTLYLQMTSLRAEDTAVYYCANGMTGTTDVDYWGQGTLVTVSS

SEQ ID NO: 148 - protein, 119 aa, Artificial Sequence
  PR002061 VH
EVQLLESGGGLVQPGGSLRLSCAASGFTFSSYAMSWVRQAPGKGLEWVSAISGRGGSTYYADSVRGRFTISRDNSKNTLYLQMNSLRAEDTAVYYCANGMTGTTDVDYWGQGTLVTVSS

SEQ ID NO: 149 - protein, 119 aa, Artificial Sequence
  PR002062 VH
EVQLLESGGGLVQPGGSLRLSCAASGFTFSSYAMSWVRQAPGKGQEWVSAISGSGGSTFYADSVRGRFTISRDNSKNTLYLQMSSLRAEDTAVYYCANGMTGTTDVDYWGQGTLVTVSS

SEQ ID NO: 150 - protein, 119 aa, Artificial Sequence
  PR002063 VH
EVQLLESGGGLVQPGGSLRLSCAASGFTFSSYAMSWVRQAPGRGLEWVSAISGGGGSTFYADSVRGRFTISRDNSKNTLYLQMSSLRAEDTAVYYCANGMTGTTDVDYWGQGTRVTVSS

SEQ ID NO: 151 - protein, 119 aa, Artificial Sequence
  PR002064 VH
EVQLVETGGGLVQPGGSLRLSCAASGFTFSSYAMSWVRQAPGKGQEWVSAISGSGGSTFYADSVRGRFTISRDNSKNTLYLQMSSLRAEDTAVYYCANGMTGTTDVDYWGQGTLVTVSS

SEQ ID NO: 152 - protein, 119 aa, Artificial Sequence
  PR002065 VH
EVQLVESGGGLVQPGGSLRLSCAASGFTFSSYAMSWVRQAPGKGLEWVSAISGRGDITFYADSVRGRFTISRDNSKNTLYLQMNSLRAEDTALYYCANGMTGSTDVDFWGQGTLVTVSS

SEQ ID NO: 153 - protein, 119 aa, Artificial Sequence
  PR002066 VH
EVQLLESGGGLVQPGGSLRLSCIASGFTFSSYAMSWVRQAPGKGLEWVSAISGRGGSTFYADSVRGRFTISRDNSKNTLYLQMNSLRAEDTAVYYCANGMTGTTDVDYWGQGTQVTVSS

SEQ ID NO: 154 - protein, 119 aa, Artificial Sequence
  PR002067 VH
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSS

SEQ ID NO: 155 - protein, 119 aa, Artificial Sequence
  PR002068 VH
EVQLLESGGGLVQPGGSLRLSCAASGFSFSSYAMTWVRQAPGKGLEWVSAISGGGGSIFYADSVRGRFTISRDNSKNTLYLQMNSLRAEDTAVYYCANGMTGSTDVDYWGQGTQVTVSS

SEQ ID NO: 156 - protein, 119 aa, Artificial Sequence
  PR002069 VH
EVQLLESGGGLVQPGGSLRLSCAASGFTFSSYAMSWVRQAPGKGREWVSAISGGGGNTFYADSVRGRFTISRDNSKNTLYLQMNSLRAEDTAVYYCANGVTGTTDVDYWGQGTLVTVSS

SEQ ID NO: 157 - protein, 119 aa, Artificial Sequence
  PR002070 VH
EVQLLESGGGLVQPGGSLRLSCIASGFSFSSYAMSWVRQAPGKGLEWVSAISGRGGSTFYADSVRGRFNISRDNSKNTLYLQMNSLRVEDTAVYYCANGMTGTTDVDYWGQGTLVTVSS

SEQ ID NO: 158 - protein, 119 aa, Artificial Sequence
  PR002071 VH
EVQLVESGGGLVQPGGSLRLSCAASGFTFSSYAMSWVRQAPGKGLEWVSAISGGGGSIFYADSVRGRFTISRDNSKNTLYLQMNSLRAEDTAVYYCANGMTGTTDVDYWGQGTLVTVSS

SEQ ID NO: 159 - protein, 119 aa, Artificial Sequence
  PR002072 VH
EVQLLESGGGLVQPGGSLRLSCAASGFTFSSYAMSWVRQASGKGLEWISAISGSGGSTYYADSVRGRFTISRDNSKNTLYLQMNSLRADDTAVYYCANGMTGTTDVDFWGQGTPVTVSS

SEQ ID NO: 160 - protein, 118 aa, Artificial Sequence
  PR002073 VH
EVQLVESGGGLVQPGGSLRLSCAASGFTFSSYAMSWVRQAPGKGLEWVSAISGGSGSTFYADSVRGRFTISRDNSKNTLYLQMNSLRAEDTAVYYCADGVTGTDFDFWGQGTLVTVSS

SEQ ID NO: 161 - protein, 118 aa, Artificial Sequence
  PR002074 VH
EVQLLESGGGLVQPGGSLRLSCAASGFTFSSYAMSWVRQAPGKGLEWVSGISGGGNNTFYADSVRGRFTISRDNSKNTLYLQMNSLRAEDTAVYYCMRGWELPLLENWGQGTLVTVSS

SEQ ID NO: 162 - protein, 119 aa, Artificial Sequence
  PR002075 VH
EVQLVETGGGLIQPGGSLRLSCAASGFTFSSYAMSWVRQAPGKGLEWVSAISGSGGNTYYADSVRGRFTISRDNSKNTLYLQMNSLRAEDTAVYYCANGITGTTDVDYWGQGTRVTVSS

SEQ ID NO: 163 - protein, 119 aa, Artificial Sequence
  PR002076 VH
EVQLLESGGGLVQPGGSLRLSCAASGFTFSSYAMSWVRQAPGKGLEWVSAISGSGGNTYYADSVRGRFTISRDNSKNTLYLQMNSLRAEDTAVYYCANGMTGTTDVDYWGQGTRVTVSS

SEQ ID NO: 164 - protein, 119 aa, Artificial Sequence
  PR002077 VH
EVQLLESGGGLVQPGGSLRLSCAASGFTFSSYAMSWVRQAPGKGLEWVSAISGRGNITFYADSVRGRFTISRDNSKNTLYLQMNSLRAEDTAVYYCANGITGTTDVDYWGQGTLVTVSS

SEQ ID NO: 165 - protein, 119 aa, Artificial Sequence
  PR002408 VH
QVQLVESGGGVVQPGRSLRLSCAASGFTFRSFGMHWVRQAPGKGLEWVAVISYDASNEYYADSVKGRFIISRDNSKDTLYLQMNSLRAGDTAVYYCAKGGGLRWYFAYWGQGTLVTVSS

SEQ ID NO: 166 - protein, 120 aa, Artificial Sequence
  PR002726 VH
EVQLLESGGGLVQPGGSLRLSCAASGFTFSSFVMSWVRQAPGKGLEWVSTISGSGRSTYYADSVKGRFTISRDNSKNTLHLQMNSLRAEDTAVYYCAKDAAAAGTKFDYWGQGTLVTVSS

SEQ ID NO: 167 - protein, 117 aa, Artificial Sequence
  PR003475 VH
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 168 - protein, 119 aa, Artificial Sequence
  PR005362 VH
EVQLLESGGGLVQPGGSLRLSCAASGLPFDCYAMSWVRQAPGKGLEWVSAISGRGGSTFYADSVRGRFTISRDNSKNTLYLQMNSLRAEDTAMYYCVDGTTGTTDVDYWGQGTLVTVSS

SEQ ID NO: 169 - protein, 119 aa, Artificial Sequence
  PR005363 VH
EVQLLESGGGLVQPGGSLRLSCAASGLPFDSYAMSWVRQAPGKGLEWVSAISGRGGSTFYADSVRGRFTISRDNSKNTLYLQMNSLRAEDTAMYYCVDGTTGTTDVDYWGQGTLVTVSS

SEQ ID NO: 170 - protein, 119 aa, Artificial Sequence
  PR005364 VH
EVQLLESGGGLVQPGGSLRLSCAASGFTFSSYAMSWVRQAPGKGLEWVSAISGRGGQIFYADSVRGRFTISRDNSKNTLYLQMNSLRAEDTAMYYCVDGTTGTTDVDYWGQGTLVTVSS

SEQ ID NO: 171 - protein, 119 aa, Artificial Sequence
  PR005365 VH
EVQLLESGGGLVQPGGSLRLSCAASGFTFSSYAMSWVRQAPGKGLEWVSAISGLGGQIFYADSVRGRFTISRDNSKNTLYLQMNSLRAEDTAMYYCVDGTTGTTDVDYWGQGTLVTVSS

SEQ ID NO: 172 - protein, 119 aa, Artificial Sequence
  PR005366 VH
EVQLLESGGGLVQPGGSLRLSCAASGFTFSSYAMSWVRQAPGKGLEWVSAISGRSGQIFYADSVRGRFTISRDNSKNTLYLQMNSLRAEDTAMYYCVDGTTGTTDVDYWGQGTLVTVSS

SEQ ID NO: 173 - protein, 119 aa, Artificial Sequence
  PR005367 VH
EVQLLESGGGLVQPGGSLRLSCAASGFTFSSYAMSWVRQAPGKGLEWVSAISGGGGSIFYADSVRGRFTISRDNSKNTLYLQMNSLRAEDTAMYYCVDGTTGTTDVDYWGQGTLVTVSS

SEQ ID NO: 174 - protein, 119 aa, Artificial Sequence
  PR005368 VH
EVQLLESGGGLVQPGGSLRLSCAASGFTFSSYAMSWVRQAPGKGLEWVSAISGLGGSIFYADSVRGRFTISRDNSKNTLYLQMNSLRAEDTAMYYCVDGTTGTTDVDYWGQGTLVTVSS

SEQ ID NO: 175 - protein, 119 aa, Artificial Sequence
  PR005369 VH
EVQLLESGGGLVQPGGSLRLSCAASGFTFSSYAMSWVRQAPGKGLEWVSAITGRGGQIFYADSVRGRFTISRDNSKNTLYLQMNSLRAEDTAMYYCVDGTTGTTDVDYWGQGTLVTVSS

SEQ ID NO: 176 - protein, 119 aa, Artificial Sequence
  PR005370 VH
EVQLLESGGGLVQPGGSLRLSCAASGFTFSSYAMSWVRQAPGKGLEWVSAISGHGGTIFYADSVRGRFTISRDNSKNTLYLQMNSLRAEDTAMYYCVDGTTGTTDVDYWGQGTLVTVSS

SEQ ID NO: 177 - protein, 119 aa, Artificial Sequence
  PR005371 VH
EVQLLESGGGLVQPGGSLRLSCAASGFTFSSYAMSWVRQAPGKGLEWVSAISGRGGTIFYADSVRGRFTISRDNSKNTLYLQMNSLRAEDTAMYYCVDGTTGTTDVDYWGQGTLVTVSS

SEQ ID NO: 178 - protein, 119 aa, Artificial Sequence
  PR005372 VH
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGSWDVCYWGQGTLVTVSS

SEQ ID NO: 179 - protein, 119 aa, Artificial Sequence
  PR005373 VH
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTWDVDWWGQGTLVTVSS

SEQ ID NO: 180 - protein, 119 aa, Artificial Sequence
  PR005374 VH
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGSWDVDWWGQGTLVTVSS

SEQ ID NO: 181 - protein, 119 aa, Artificial Sequence
  PR005375 VH
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGSYDVDWWGQGTLVTVSS

SEQ ID NO: 182 - protein, 119 aa, Artificial Sequence
  PR005376 VH
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGSTDVDWWGQGTLVTVSS

SEQ ID NO: 183 - protein, 119 aa, Artificial Sequence
  PR005377 VH
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTWDVDYWGQGTLVTVSS

SEQ ID NO: 184 - protein, 119 aa, Artificial Sequence
  PR005378 VH
EVQLLESGGGLVQPGGSLRLSCAASGFTFSSYAMSWVRQAPGKGLEWVSAISGRGGQIFYADSVRGRFTISRDNSKNTLYLQMNSLRAEDTAMYYCVDGTTGTWDVDWWGQGTLVTVSS

SEQ ID NO: 185 - protein, 119 aa, Artificial Sequence
  PR005379 VH
EVQLLESGGGLVQPGGSLRLSCAASGFTFSSYAMSWVRQAPGKGLEWVSAISGRGGQIFYADSVRGRFTISRDNSKNTLYLQMNSLRAEDTAMYYCVDGTTGSYDVDWWGQGTLVTVSS

SEQ ID NO: 186 - protein, 119 aa, Artificial Sequence
  PR005380 VH
EVQLLESGGGLVQPGGSLRLSCAASGFTFSSYAMSWVRQAPGKGLEWVSAISGLGGQIFYADSVRGRFTISRDNSKNTLYLQMNSLRAEDTAMYYCVDGTTGTWDVDWWGQGTLVTVSS

SEQ ID NO: 187 - protein, 119 aa, Artificial Sequence
  PR005381 VH
EVQLLESGGGLVQPGGSLRLSCAASGFTFSSYAMSWVRQAPGKGLEWVSAISGLGGQIFYADSVRGRFTISRDNSKNTLYLQMNSLRAEDTAMYYCVDGTTGSYDVDWWGQGTLVTVSS

SEQ ID NO: 188 - protein, 119 aa, Artificial Sequence
  PR005382 VH
EVQLLESGGGLVQPGGSLRLSCAASGFTFSSYAMSWVRQAPGKGLEWVSAISGRSGQIFYADSVRGRFTISRDNSKNTLYLQMNSLRAEDTAMYYCVDGTTGTWDVDWWGQGTLVTVSS

SEQ ID NO: 189 - protein, 119 aa, Artificial Sequence
  PR005383 VH
EVQLLESGGGLVQPGGSLRLSCAASGFTFSSYAMSWVRQAPGKGLEWVSAISGRSGQIFYADSVRGRFTISRDNSKNTLYLQMNSLRAEDTAMYYCVDGTTGSYDVDWWGQGTLVTVSS

SEQ ID NO: 190 - protein, 119 aa, Artificial Sequence
  PR005384 VH
EVQLLESGGGLVQPGGSLRLSCAASGLPFSSYAMSWVRQAPGKGLEWVSAISGRGGQIFYADSVRGRFTISRDNSKNTLYLQMNSLRAEDTAMYYCVDGTTGTTDVDYWGQGTLVTVSS

SEQ ID NO: 191 - protein, 119 aa, Artificial Sequence
  PR005385 VH
EVQLLESGGGLVQPGGSLRLSCAASGLTFDSYAMSWVRQAPGKGLEWVSAISGRGGQIFYADSVRGRFTISRDNSKNTLYLQMNSLRAEDTAMYYCVDGTTGTTDVDYWGQGTLVTVSS

SEQ ID NO: 192 - protein, 119 aa, Artificial Sequence
  PR005386 VH
EVQLLESGGGLVQPGGSLRLSCAASGFPFDSYAMSWVRQAPGKGLEWVSAISGRGGQIFYADSVRGRFTISRDNSKNTLYLQMNSLRAEDTAMYYCVDGTTGTTDVDYWGQGTLVTVSS

SEQ ID NO: 193 - protein, 119 aa, Artificial Sequence
  PR005387 VH
EVQLLESGGGLVQPGGSLRLSCAASGLPFDSYAMSWVRQAPGKGLEWVSAISGRGGQIFYADSVRGRFTISRDNSKNTLYLQMNSLRAEDTAMYYCVDGTTGTTDVDYWGQGTLVTVSS

SEQ ID NO: 194 - protein, 119 aa, Artificial Sequence
  PR005388 VH
EVQLLESGGGLVQPGGSLRLSCAASGFTFSSYAMSWVRQAPGKGLEWVSAISGRGGQIFYADSVRGRFTISRDNSKNTLYLQMNSLRAEDTAMYYCVDGTTGTWDVDYWGQGTLVTVSS

SEQ ID NO: 195 - protein, 119 aa, Artificial Sequence
  PR005389 VH
EVQLLESGGGLVQPGGSLRLSCAASGLPFSSYAMSWVRQAPGKGLEWVSAISGRGGQIFYADSVRGRFTISRDNSKNTLYLQMNSLRAEDTAMYYCVDGTTGTWDVDYWGQGTLVTVSS

SEQ ID NO: 196 - protein, 119 aa, Artificial Sequence
  PR005390 VH
EVQLLESGGGLVQPGGSLRLSCAASGLTFDSYAMSWVRQAPGKGLEWVSAISGRGGQIFYADSVRGRFTISRDNSKNTLYLQMNSLRAEDTAMYYCVDGTTGTWDVDYWGQGTLVTVSS

SEQ ID NO: 197 - protein, 119 aa, Artificial Sequence
  PR005391 VH
EVQLLESGGGLVQPGGSLRLSCAASGFPFDSYAMSWVRQAPGKGLEWVSAISGRGGQIFYADSVRGRFTISRDNSKNTLYLQMNSLRAEDTAMYYCVDGTTGTWDVDYWGQGTLVTVSS

SEQ ID NO: 198 - protein, 119 aa, Artificial Sequence
  PR005392 VH
EVQLLESGGGLVQPGGSLRLSCAASGLPFDSYAMSWVRQAPGKGLEWVSAISGRGGQIFYADSVRGRFTISRDNSKNTLYLQMNSLRAEDTAMYYCVDGTTGTWDVDYWGQGTLVTVSS

SEQ ID NO: 199 - protein, 107 aa, Artificial Sequence
  PR000265 VL
DIQMTQSPSTLSASVGDRVTVTCRASQSIYIWLAWYQQKPGKAPNLLIYKASSLETGVPSRFSGSGSGTEFTLTISSLQPDDFATYYCQQYYGSSRTFGQGTKVEIK

SEQ ID NO: 200 - protein, 108 aa, Artificial Sequence
  PR001081 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPPITFGQGTRLEIK

SEQ ID NO: 201 - protein, 107 aa, Artificial Sequence
  PR001331 VL
DIQMTQSPSSLSASIGDRVTITCRASQSINSYLNWYQQKPGKAPKLLIYTASSLLSGVPSRFSASGSWTDFSLTISSLQPEDFATYYCQQSFSTPYTFGQGTNLEIK

SEQ ID NO: 202 - protein, 108 aa, Artificial Sequence
  PR002408 VL
EIVMTQSPATLSVSPGERATLSCRASQSISSNLGWYQQKPGQAPRLLIYGASTRATGIPARFSGSGSGTEFTLTISSLQSEDFAVYYCQQYQSWPPLTFGGGTKVEIK

SEQ ID NO: 203 - protein, 107 aa, Artificial Sequence
  PR002726 VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 204 - protein, 107 aa, Artificial Sequence
  PR003475 VL
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 205 - protein, 449 aa, Artificial Sequence
  PR000265 HC
EVQLVESGGGLVQPGGSLRLSCAASGFTFSSYWMSWVRQAPGKGLEWVANIKQEGSEKYYVDSVKGRFTISRDNAKNSLYLQMNSLRAEDTAVYYCARDRAVAGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 448 aa, Artificial Sequence
  PR001081 HC
QVQLVESGGGVVQPGRSLRLSCAASGFTFSRYDMNWVRQAPGKGLEWVAVIWFDGSNKYYADSVKGRFAISRDNSKNTLYLQMNSLRAEDTAVYYCAREMAAAGFYLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 452 aa, Artificial Sequence
  PR001331 HC
QVQLVESGGGVVQPGRSLRLSCSASGFTFINYGMNWVRQAPGKGLEWVAVIWYDGSNKYYADSVRGRFTISRDNSKNTLYLQMNSLRAEDTAVYYCARGPGYGGHSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 351 aa, Artificial Sequence
  PR002055 HC
EVQLLESGGGLVQPGGSLRLSCAASGLTFSSYAMSWVRQAPGKGLEWISAISGGGGSTYYADSVRGRFTISRDNSKNTLYLQMNSLRAEDTAVYYCANGMTGSTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 351 aa, Artificial Sequence
  PR002056 HC
EVQLLESGGGLVQPGGSLRLSCAASGFTFSSYAMSWVRQAPGKGLEWVSAISGGGGSIFYADSVRGRFTISRDNSKNTLYLQMNSLRAEDTAVYYCANGMTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 351 aa, Artificial Sequence
  PR002057 HC
EVQLLESGGGLVQPGGSLRLSCAASGFIFSSYAMSWVRQAPGKGLEWVSAISGGGGSTFYADSVRGRFTISRDNSKNTLYLQMNSLRAEDTAVYYCADGMTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 350 aa, Artificial Sequence
  PR002058 HC
EVQLLESGGGLVQPGGSLRLSCAASGFTFSSYAMSWVRQAPGKGLEWVSAISGGSGSTFYADSVRGRFTISRDNSKNTLYLQMNSLRAEDTAVYYCADGVTGTDFDFWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 351 aa, Artificial Sequence
  PR002059 HC
EVQLLESGGGLVQPGGSLRLSCVVSGLTFSSYAMSWVRQAPGKGREWVSAISGRGGSTFYADSVRGRFTISRDNSKNTLYLQMNSLRAEDTAVYYCANGMTGSTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 351 aa, Artificial Sequence
  PR002060 HC
EVQLLESGGGLVQPGGSLRLSCAASGFTFSDYAMSWVRQAPGKGLEWVSAISGRGGSTYYADSVRGRFTISRDNSKNTLYLQMTSLRAEDTAVYYCANGMTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 351 aa, Artificial Sequence
  PR002061 HC
EVQLLESGGGLVQPGGSLRLSCAASGFTFSSYAMSWVRQAPGKGLEWVSAISGRGGSTYYADSVRGRFTISRDNSKNTLYLQMNSLRAEDTAVYYCANGMTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 351 aa, Artificial Sequence
  PR002062 HC
EVQLLESGGGLVQPGGSLRLSCAASGFTFSSYAMSWVRQAPGKGQEWVSAISGSGGSTFYADSVRGRFTISRDNSKNTLYLQMSSLRAEDTAVYYCANGMTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 351 aa, Artificial Sequence
  PR002063 HC
EVQLLESGGGLVQPGGSLRLSCAASGFTFSSYAMSWVRQAPGRGLEWVSAISGGGGSTFYADSVRGRFTISRDNSKNTLYLQMSSLRAEDTAVYYCANGMTGTTDVDYWGQGTRVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 351 aa, Artificial Sequence
  PR002064 HC
EVQLVETGGGLVQPGGSLRLSCAASGFTFSSYAMSWVRQAPGKGQEWVSAISGSGGSTFYADSVRGRFTISRDNSKNTLYLQMSSLRAEDTAVYYCANGMTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 351 aa, Artificial Sequence
  PR002065 HC
EVQLVESGGGLVQPGGSLRLSCAASGFTFSSYAMSWVRQAPGKGLEWVSAISGRGDITFYADSVRGRFTISRDNSKNTLYLQMNSLRAEDTALYYCANGMTGSTDVDFWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 351 aa, Artificial Sequence
  PR002066 HC
EVQLLESGGGLVQPGGSLRLSCIASGFTFSSYAMSWVRQAPGKGLEWVSAISGRGGSTFYADSVRGRFTISRDNSKNTLYLQMNSLRAEDTAVYYCANGMTGTTDVDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 351 aa, Artificial Sequence
  PR002067 HC
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 351 aa, Artificial Sequence
  PR002068 HC
EVQLLESGGGLVQPGGSLRLSCAASGFSFSSYAMTWVRQAPGKGLEWVSAISGGGGSIFYADSVRGRFTISRDNSKNTLYLQMNSLRAEDTAVYYCANGMTGSTDVDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 351 aa, Artificial Sequence
  PR002069 HC
EVQLLESGGGLVQPGGSLRLSCAASGFTFSSYAMSWVRQAPGKGREWVSAISGGGGNTFYADSVRGRFTISRDNSKNTLYLQMNSLRAEDTAVYYCANGVTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 351 aa, Artificial Sequence
  PR002070 HC
EVQLLESGGGLVQPGGSLRLSCIASGFSFSSYAMSWVRQAPGKGLEWVSAISGRGGSTFYADSVRGRFNISRDNSKNTLYLQMNSLRVEDTAVYYCANGMTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 351 aa, Artificial Sequence
  PR002071 HC
EVQLVESGGGLVQPGGSLRLSCAASGFTFSSYAMSWVRQAPGKGLEWVSAISGGGGSIFYADSVRGRFTISRDNSKNTLYLQMNSLRAEDTAVYYCANGMTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 351 aa, Artificial Sequence
  PR002072 HC
EVQLLESGGGLVQPGGSLRLSCAASGFTFSSYAMSWVRQASGKGLEWISAISGSGGSTYYADSVRGRFTISRDNSKNTLYLQMNSLRADDTAVYYCANGMTGTTDVDFWGQGTPVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 350 aa, Artificial Sequence
  PR002073 HC
EVQLVESGGGLVQPGGSLRLSCAASGFTFSSYAMSWVRQAPGKGLEWVSAISGGSGSTFYADSVRGRFTISRDNSKNTLYLQMNSLRAEDTAVYYCADGVTGTDFDFWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 350 aa, Artificial Sequence
  PR002074 HC
EVQLLESGGGLVQPGGSLRLSCAASGFTFSSYAMSWVRQAPGKGLEWVSGISGGGNNTFYADSVRGRFTISRDNSKNTLYLQMNSLRAEDTAVYYCMRGWELPLLENWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 351 aa, Artificial Sequence
  PR002075 HC
EVQLVETGGGLIQPGGSLRLSCAASGFTFSSYAMSWVRQAPGKGLEWVSAISGSGGNTYYADSVRGRFTISRDNSKNTLYLQMNSLRAEDTAVYYCANGITGTTDVDYWGQGTRVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 351 aa, Artificial Sequence
  PR002076 HC
EVQLLESGGGLVQPGGSLRLSCAASGFTFSSYAMSWVRQAPGKGLEWVSAISGSGGNTYYADSVRGRFTISRDNSKNTLYLQMNSLRAEDTAVYYCANGMTGTTDVDYWGQGTRVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 351 aa, Artificial Sequence
  PR002077 HC
EVQLLESGGGLVQPGGSLRLSCAASGFTFSSYAMSWVRQAPGKGLEWVSAISGRGNITFYADSVRGRFTISRDNSKNTLYLQMNSLRAEDTAVYYCANGITGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 449 aa, Artificial Sequence
  PR002408 HC
QVQLVESGGGVVQPGRSLRLSCAASGFTFRSFGMHWVRQAPGKGLEWVAVISYDASNEYYADSVKGRFIISRDNSKDTLYLQMNSLRAGDTAVYYCAKGGGLRWYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 450 aa, Artificial Sequence
  PR002726 HC
EVQLLESGGGLVQPGGSLRLSCAASGFTFSSFVMSWVRQAPGKGLEWVSTISGSGRSTYYADSVKGRFTISRDNSKNTLHLQMNSLRAEDTAVYYCAKDAAAAGTKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 447 aa, Artificial Sequence
  PR003475 HC
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 351 aa, Artificial Sequence
  PR005362 HC
EVQLLESGGGLVQPGGSLRLSCAASGLPFDCYAMSWVRQAPGKGLEWVSAISGRGGSTFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 351 aa, Artificial Sequence
  PR005363 HC
EVQLLESGGGLVQPGGSLRLSCAASGLPFDSYAMSWVRQAPGKGLEWVSAISGRGGSTFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 351 aa, Artificial Sequence
  PR005364 HC
EVQLLESGGGLVQPGGSLRLSCAASGFTFSSYAMSWVRQAPGKGLEWVSAISGRGGQIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 351 aa, Artificial Sequence
  PR005365 HC
EVQLLESGGGLVQPGGSLRLSCAASGFTFSSYAMSWVRQAPGKGLEWVSAISGLGGQIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 351 aa, Artificial Sequence
  PR005366 HC
EVQLLESGGGLVQPGGSLRLSCAASGFTFSSYAMSWVRQAPGKGLEWVSAISGRSGQIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 351 aa, Artificial Sequence
  PR005367 HC
EVQLLESGGGLVQPGGSLRLSCAASGFTFSSYAMSWVRQAPGKGLEWVSAISGGGGSIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 351 aa, Artificial Sequence
  PR005368 HC
EVQLLESGGGLVQPGGSLRLSCAASGFTFSSYAMSWVRQAPGKGLEWVSAISGLGGSIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 351 aa, Artificial Sequence
  PR005369 HC
EVQLLESGGGLVQPGGSLRLSCAASGFTFSSYAMSWVRQAPGKGLEWVSAITGRGGQIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 351 aa, Artificial Sequence
  PR005370 HC
EVQLLESGGGLVQPGGSLRLSCAASGFTFSSYAMSWVRQAPGKGLEWVSAISGHGGTIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 351 aa, Artificial Sequence
  PR005371 HC
EVQLLESGGGLVQPGGSLRLSCAASGFTFSSYAMSWVRQAPGKGLEWVSAISGRGGTIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 351 aa, Artificial Sequence
  PR005372 HC
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGSWDVCYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 351 aa, Artificial Sequence
  PR005373 HC
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTWDVDWWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - protein, 351 aa, Artificial Sequence
  PR005374 HC
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGSWDVDWWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 351 aa, Artificial Sequence
  PR005375 HC
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGSYDVDWWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 351 aa, Artificial Sequence
  PR005376 HC
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGSTDVDWWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 351 aa, Artificial Sequence
  PR005377 HC
EVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTWDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 351 aa, Artificial Sequence
  PR005378 HC
EVQLLESGGGLVQPGGSLRLSCAASGFTFSSYAMSWVRQAPGKGLEWVSAISGRGGQIFYADSVRGRFTISRDNSKNTLYLQMNSLRAEDTAMYYCVDGTTGTWDVDWWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 351 aa, Artificial Sequence
  PR005379 HC
EVQLLESGGGLVQPGGSLRLSCAASGFTFSSYAMSWVRQAPGKGLEWVSAISGRGGQIFYADSVRGRFTISRDNSKNTLYLQMNSLRAEDTAMYYCVDGTTGSYDVDWWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 252 - protein, 351 aa, Artificial Sequence
  PR005380 HC
EVQLLESGGGLVQPGGSLRLSCAASGFTFSSYAMSWVRQAPGKGLEWVSAISGLGGQIFYADSVRGRFTISRDNSKNTLYLQMNSLRAEDTAMYYCVDGTTGTWDVDWWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - protein, 351 aa, Artificial Sequence
  PR005381 HC
EVQLLESGGGLVQPGGSLRLSCAASGFTFSSYAMSWVRQAPGKGLEWVSAISGLGGQIFYADSVRGRFTISRDNSKNTLYLQMNSLRAEDTAMYYCVDGTTGSYDVDWWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 351 aa, Artificial Sequence
  PR005382 HC
EVQLLESGGGLVQPGGSLRLSCAASGFTFSSYAMSWVRQAPGKGLEWVSAISGRSGQIFYADSVRGRFTISRDNSKNTLYLQMNSLRAEDTAMYYCVDGTTGTWDVDWWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - protein, 351 aa, Artificial Sequence
  PR005383 HC
EVQLLESGGGLVQPGGSLRLSCAASGFTFSSYAMSWVRQAPGKGLEWVSAISGRSGQIFYADSVRGRFTISRDNSKNTLYLQMNSLRAEDTAMYYCVDGTTGSYDVDWWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 351 aa, Artificial Sequence
  PR005384 HC
EVQLLESGGGLVQPGGSLRLSCAASGLPFSSYAMSWVRQAPGKGLEWVSAISGRGGQIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 351 aa, Artificial Sequence
  PR005385 HC
EVQLLESGGGLVQPGGSLRLSCAASGLTFDSYAMSWVRQAPGKGLEWVSAISGRGGQIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 351 aa, Artificial Sequence
  PR005386 HC
EVQLLESGGGLVQPGGSLRLSCAASGFPFDSYAMSWVRQAPGKGLEWVSAISGRGGQIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 351 aa, Artificial Sequence
  PR005387 HC
EVQLLESGGGLVQPGGSLRLSCAASGLPFDSYAMSWVRQAPGKGLEWVSAISGRGGQIFYADSVRGRFTISRDNSKNTLYLQMNSLRAEDTAMYYCVDGTTGTTDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 351 aa, Artificial Sequence
  PR005388 HC
EVQLLESGGGLVQPGGSLRLSCAASGFTFSSYAMSWVRQAPGKGLEWVSAISGRGGQIFYADSVRGRFTISRDNSKNTLYLQMNSLRAEDTAMYYCVDGTTGTWDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - protein, 351 aa, Artificial Sequence
  PR005389 HC
EVQLLESGGGLVQPGGSLRLSCAASGLPFSSYAMSWVRQAPGKGLEWVSAISGRGGQIFYADSVRGRFTISRDNSKNTLYLQMNSLRAEDTAMYYCVDGTTGTWDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 351 aa, Artificial Sequence
  PR005390 HC
EVQLLESGGGLVQPGGSLRLSCAASGLTFDSYAMSWVRQAPGKGLEWVSAISGRGGQIFYADSVRGRFTISRDNSKNTLYLQMNSLRAEDTAMYYCVDGTTGTWDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - protein, 351 aa, Artificial Sequence
  PR005391 HC
EVQLLESGGGLVQPGGSLRLSCAASGFPFDSYAMSWVRQAPGKGLEWVSAISGRGGQIFYADSVRGRFTISRDNSKNTLYLQMNSLRAEDTAMYYCVDGTTGTWDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 351 aa, Artificial Sequence
  PR005392 HC
EVQLLESGGGLVQPGGSLRLSCAASGLPFDSYAMSWVRQAPGKGLEWVSAISGRGGQIFYADSVRGRFTISRDNSKNTLYLQMNSLRAEDTAMYYCVDGTTGTWDVDYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 265 - protein, 214 aa, Artificial Sequence
  PR000265 LC, PR003789 Chain
DIQMTQSPSTLSASVGDRVTVTCRASQSIYIWLAWYQQKPGKAPNLLIYKASSLETGVPSRFSGSGSGTEFTLTISSLQPDDFATYYCQQYYGSSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 266 - protein, 215 aa, Artificial Sequence
  PR001081 LC, PR004283 Chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 214 aa, Artificial Sequence
  PR001331 LC, PR004284 Chain
DIQMTQSPSSLSASIGDRVTITCRASQSINSYLNWYQQKPGKAPKLLIYTASSLLSGVPSRFSASGSWTDFSLTISSLQPEDFATYYCQQSFSTPYTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 215 aa, Artificial Sequence
  PR002408 LC, PR004276 Chain
EIVMTQSPATLSVSPGERATLSCRASQSISSNLGWYQQKPGQAPRLLIYGASTRATGIPARFSGSGSGTEFTLTISSLQSEDFAVYYCQQYQSWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 214 aa, Artificial Sequence
  PR002726 LC, PR004285 Chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - protein, 214 aa, Artificial Sequence
  PR003475 LC
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 583 aa, Artificial Sequence
  PR003789 Chain
EVQLVESGGGLVQPGGSLRLSCAASGFTFSSYWMSWVRQAPGKGLEWVANIKQEGSEKYYVDSVKGRFTISRDNAKNSLYLQMNSLRAEDTAVYYCARDRAVAGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAEPKSSDKTHTPPPPPEVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSS

SEQ ID NO: 272 - protein, 584 aa, Artificial Sequence
  PR004276 Chain
QVQLVESGGGVVQPGRSLRLSCAASGFTFRSFGMHWVRQAPGKGLEWVAVISYDASNEYYADSVKGRFIISRDNSKDTLYLQMNSLRAGDTAVYYCAKGGGLRWYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGEPKSSDKTHTPPPPPRTEVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSS

SEQ ID NO: 273 - protein, 222 aa, Artificial Sequence
  PR004277 Chain
QVQLVESGGGVVQPGRSLRLSCAASGFTFRSFGMHWVRQAPGKGLEWVAVISYDASNEYYADSVKGRFIISRDNSKDTLYLQMNSLRAGDTAVYYCAKGGGLRWYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 274 - protein, 581 aa, Artificial Sequence
  PR004277 Chain
EIVMTQSPATLSVSPGERATLSCRASQSISSNLGWYQQKPGQAPRLLIYGASTRATGIPARFSGSGSGTEFTLTISSLQSEDFAVYYCQQYQSWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSSDKTHTPPPPPEVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 275 - protein, 583 aa, Artificial Sequence
  PR004283 Chain
QVQLVESGGGVVQPGRSLRLSCAASGFTFSRYDMNWVRQAPGKGLEWVAVIWFDGSNKYYADSVKGRFAISRDNSKNTLYLQMNSLRAEDTAVYYCAREMAAAGFYLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGEPKSSDKTHTPPPPPRTEVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSS

SEQ ID NO: 276 - protein, 587 aa, Artificial Sequence
  PR004284 Chain
QVQLVESGGGVVQPGRSLRLSCSASGFTFINYGMNWVRQAPGKGLEWVAVIWYDGSNKYYADSVRGRFTISRDNSKNTLYLQMNSLRAEDTAVYYCARGPGYGGHSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGEPKSSDKTHTPPPPPRTEVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSS

SEQ ID NO: 277 - protein, 585 aa, Artificial Sequence
  PR004285 Chain
EVQLLESGGGLVQPGGSLRLSCAASGFTFSSFVMSWVRQAPGKGLEWVSTISGSGRSTYYADSVKGRFTISRDNSKNTLHLQMNSLRAEDTAVYYCAKDAAAAGTKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGEPKSSDKTHTPPPPPRTEVQLLESGGGLVQPGGSLRLSCAASGFTFSSYAMSWVRQAPGKGLEWVSAISGRGGSTFYADSVRGRFTISRDNSKNTLYLQMNSLRAEDTAMYYCVDGTTGTTDVDYWGQGTLVTVSS

SEQ ID NO: 278 - protein, 4 aa, Artificial Sequence
  GS_4
GSGS

SEQ ID NO: 279 - protein, 5 aa, Artificial Sequence
  GS_5
GGGGS

SEQ ID NO: 280 - protein, 7 aa, Artificial Sequence
  GS_7
GGGGSGS

SEQ ID NO: 281 - protein, 15 aa, Artificial Sequence
  GS_15
GGGGSGGGGSGGGGS

SEQ ID NO: 282 - protein, 20 aa, Artificial Sequence
  GS_20
GGGGSGGGGSGGGGSGGGGS

SEQ ID NO: 283 - protein, 25 aa, Artificial Sequence
  GS_25
GGGGSGGGGSGGGGSGGGGSGGGGS

SEQ ID NO: 284 - protein, 15 aa, Artificial Sequence
  human IgG1 hinge
EPKSCDKTHTCPPCP

SEQ ID NO: 285 - protein, 15 aa, Artificial Sequence
  human IgG1 hinge (C220S)
EPKSSDKTHTCPPCP

SEQ ID NO: 286 - protein, 15 aa, Artificial Sequence
  H1_15
EPKSSDKTHTPPPPP

SEQ ID NO: 287 - protein, 15 aa, Artificial Sequence
  G5-LH
GGGGGDKTHTCPPCP

SEQ ID NO: 288 - protein, 17 aa, Artificial Sequence
  H1_15-RT
EPKSSDKTHTPPPPPRT

SEQ ID NO: 289 - protein, 18 aa, Artificial Sequence
  L-GS_15-RT
LGGGGSGGGGSGGGGSRT

SEQ ID NO: 290 - protein, 18 aa, Artificial Sequence
  L-H1_15-RT
LEPKSSDKTHTPPPPPRT

SEQ ID NO: 291 - protein, 19 aa, Artificial Sequence
  KL-H1_15-RT
KLEPKSSDKTHTPPPPPRT

SEQ ID NO: 292 - protein, 19 aa, Artificial Sequence
  KL-H1_15-AS
KLEPKSSDKTHTPPPPPAS

SEQ ID NO: 293 - protein, 9 aa, Artificial Sequence
  RT-GS_5-KL
RTGGGGSKL

SEQ ID NO: 294 - protein, 19 aa, Artificial Sequence
  RT-GS_15-KL
RTGGGGSGGGGSGGGGSKL

SEQ ID NO: 295 - protein, 29 aa, Artificial Sequence
  RT-GS_25-KL
RTGGGGSGGGGSGGGGSGGGGSGGGGSKL